(19) United States
(12) Patent Application Publication
(10) Pub. No.: US 2022/0081421 A1
(43) Pub. Date: Mar. 17, 2022

(54) ANTIBACTERIAL COMPOUNDS

(71) Applicant: Forge Therapeutics, Inc., San Diego, CA (US)

(72) Inventors: Min TENG, San Diego, CA (US); Baskar NAMMALWAR, San Diego, CA (US); Xiaoming LI, San Diego, CA (US); Ian YULE, Abingdon (GB); Alastair PARKES, Abingdon (GB); Heather TYE, Abingdon (GB); Holly ATTON, Abingdon (GB); Helen WILLIAMS, Abingdon (GB); Adele FAULKNER, Abingdon (GB); Serge CONVERS-REIGNIER, Abingdon (GB); David T. PUERTA, San Diego, CA (US)

(21) Appl. No.: 17/293,358

(22) PCT Filed: Nov. 14, 2019
(86) PCT No.: PCT/US2019/061529

§ 371 Date: May 12, 2021

Related U.S. Application Data
(60) Provisional application No. 62/767,316, filed on Nov. 14, 2018.

(51) Int. Cl. C07D 403/10; A61K 9/48; A61P 31/04; C07D 213/69; C07D 239/54; C07D 237/16; C07D 405/14; C07D 401/06; C07D 401/10; C07D 401/08; C07D 401/14; C07D 498/10; C07D 413/14; C07D 405/10; C07D 487/04; C07D 471/04; C07D 493/04; C07D 309/40; C07D 413/12; C07D 403/06; C07D 403/12

(52) U.S. Cl. CPC C07D 403/10; C07D 403/12; A61P 31/04; C07D 213/69; C07D 239/54; C07D 237/16; C07D 405/14; C07D 401/06; C07D 401/10; C07D 401/08; C07D 401/14; C07D 498/10; C07D 413/14; C07D 405/10; C07D 487/04; C07D 471/04; C07D 493/04; C07D 309/40; C07D 413/12; C07D 403/06; A61K 9/4825

(57) ABSTRACT

Provided herein are heterocyclic derivative compounds and pharmaceutical compositions comprising said compounds that are useful for inhibiting the growth of gram-negative bacteria. Furthermore, the subject compounds and compositions are useful for the treatment of bacterial infection, such as urinary tract infection and the like.

### RELATED APPLICATIONS

This application claims the benefit of U.S. Provisional Patent Application No. 62/767,316 filed on Nov. 14, 2018, which is herein incorporated by reference in its entirety.

### BACKGROUND

A need exists in the medicinal arts for the effective treatment of illness caused by bacterial infection.

### BRIEF SUMMARY OF THE INVENTION

Provided herein are heterocyclic derivative compounds and pharmaceutical compositions comprising said compounds that are useful for inhibiting the growth of gram-negative bacteria. Furthermore, the subject compounds and compositions are useful for the treatment of bacterial infection, such as urinary tract infection and the like.

One embodiment provides a compound, or a pharmaceutically acceptable salt thereof, having the structure of Formula (I):

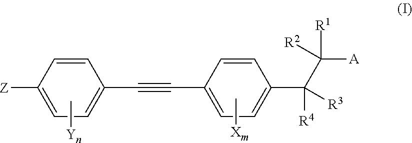

wherein:
A is

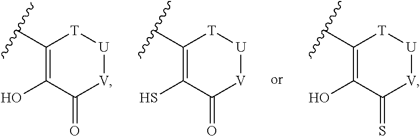

wherein the *-T-U—V— element is selected from the following:
a. *—C(R²¹)═C(R²¹)—N(R²²)—;
b. *—C(R²¹)═C(R²¹)—O—;
c. *—N(R²²)—C(R²¹)═C(R²¹)—;
d. *—N═C(R²¹)—N(R²³)—;
e. *—N═C(R²¹)—O—;
f. *—N(R²²)—N═C(R²¹)—;
g. *—C(R²¹)═N—N(R²²)—;
h. *—C(R²¹)═N—O—; and
i. *—O—C(R²¹)═C(R²¹)—;
each R²¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R²¹ groups together with the carbon atoms to which they are attached join to form a ring;
each R²² is independently H or optionally substituted alkyl; or a R²¹ group and a R²² group on adjacent atoms join to form a ring;
R²³ is optionally substituted alkyl;
n is 0-4;
m is 0-4;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₄-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
   provided that when A is

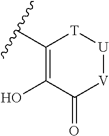

and *-T-U—V is *—O—C(R²¹)═C(R²¹)—, R³ is not H.

One embodiment provides a compound, or a pharmaceutically acceptable salt thereof, having the structure of Formula (II):

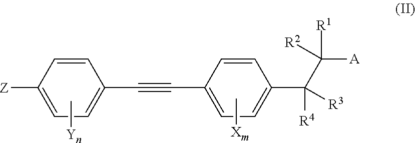

wherein:
A is

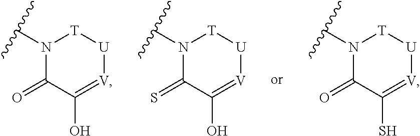

wherein the *-T-U—V— element is selected from the following:
a. *—C(R²¹)═C(R²¹)—C(R²¹)═;
b. *—N═C(R²¹)—C(R²¹)═;
c. *—C(R²¹)═N—C(R²¹)═;
d. *—C(R²¹)═C(R²¹)—N═;
e. *—N═C(R²¹)—N═;
f. *—C(R²¹)═N—N═;
g. *—N═N—C(R²¹)═; and
h. *—N═N—N═;
each R²¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R²¹ groups together with the carbon atoms to which they are attached join to form a ring;
n is 0-4;
m is 0-4;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₄-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

One embodiment provides a compound, or a pharmaceutically acceptable salt thereof, having the structure of Formula (III):

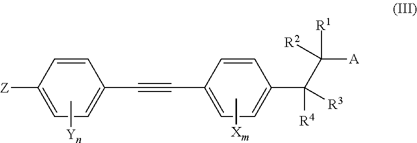

wherein:
A is

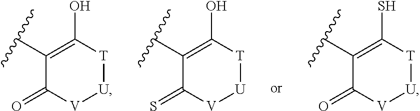

wherein the *-T-U—V— element is selected from the following:
a. *—C(R²¹)═C(R²¹)—N(R²²)—;
b. *—C(R²¹)═C(R²¹)—O—;
c. *—N(R²²)—C(R²¹)═C(R²¹)—;
d. *—N═C(R²¹)—N(R²²)—;
e. *—N═C(R²¹)—O—;
f. *—N(R²²)—N═C(R²¹)—;
g. *—C(R²¹)═N—N(R²²)—; and
h. *—C(R²¹)═N—O—;
each R²¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R²¹ groups together with the carbon atoms to which they are attached join to form a ring;
each R²² is independently H or optionally substituted alkyl; or a R²¹ group and a R²² group on adjacent atoms join to form a ring;
n is 0-4;
m is 0-4;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₄-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

One embodiment provides a compound, or a pharmaceutically acceptable salt thereof, having the structure of Formula (IV):

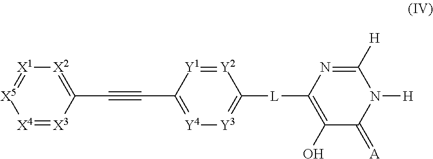

wherein:
A is O or S;
L is *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR¹R²—S—; *—N(R⁵)—C(O)—; *—C(O)—N(R⁵)—; *—N(R⁵)—S(O)₂—; *—S(O)₂—N(R⁵)—; or

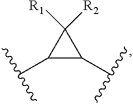

wherein the * denotes a bond to the hydroxypyrimidin-4(3H)-one ring or hydroxypyrimidin-4(3H)-thione ring;
R¹ or R³ are each independently H, optionally alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₁-C₄ alkylene)N(R¹²)—SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
each R⁵ is independently H or optionally substituted alkyl;
each X¹, X², X³, or X⁴ is N, N⁺—O⁻, or C—R⁶;
X⁵ is N, N⁺—O⁻, or C—R⁷;
each R⁶ is independently H, halogen, —OH, or optionally substituted C₁-C₃ alkyl; each Y¹, Y², Y³, or Y⁴ is N, N⁺—O⁻, or C—R⁷;
R⁷ is halogen, —OH, or optionally substituted C₁-C₃ alkyl;
Z is H, -L1-G, optionally substituted alkyl, optionally substituted carbocyclyl, optionally substituted aryl, optionally substituted heteroaryl, optionally substituted heterocyclyl;
L1 is a bond, optionally substituted C₁-C₄ alkylene, —C(O)—, or —SO₂—;
G is optionally substituted heterocyclyl, optionally substituted heteroaryl, —N(R¹³)₂, —OR¹³, —CN, —OCON(R¹³)₂, or —N(R¹⁴)CON(R¹³)₂;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
with the provision if L is *—CR¹R²—CR³R⁴—, then at least one of X¹, X², X³, X⁴, X⁵, Y¹, Y², Y³, or Y⁴ is not C.

One embodiment provides for a compound, or a pharmaceutically acceptable salt thereof, of Formula (V) wherein the structure is:

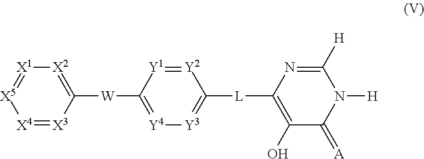

wherein:
A is O or S;
L is *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR¹R²—S—; *—N(R³)—C(O)—; *—C(O)—N(R⁵)—; *—N(R⁵)—S(O)₂—; *—S(O)₂—N(R⁵)—; or

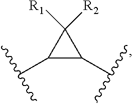

wherein the * denotes a bond to the hydroxypyrimidin-4(3H)-one ring or hydroxypyrimidin-4(3H)-thione ring;
R¹ or R³ are each independently H, optionally alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₁-C₄ alkylene)N(R¹²)—SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
each R⁵ is independently H or optionally substituted alkyl;
each X¹, X², X³, or X⁴ is N, N⁺—O⁻, or C—R⁶;
X⁵ is N, N⁺—O⁻, or C—R⁷;
each R⁶ is independently H, halogen, —OH, or optionally substituted C₁-C₃ alkyl; each Y¹, Y², Y³, or Y⁴ is N, N⁺—O⁻, or C—R⁷;
R⁷ is halogen, —OH, or optionally substituted C₁-C₃ alkyl;
Z is H, -L1-G, optionally substituted alkyl, optionally substituted carbocyclyl, optionally substituted aryl, optionally substituted heteroaryl, optionally substituted heterocyclyl;
L1 is a bond, optionally substituted C₁-C₄ alkylene, —C(O)—, or —SO₂—;
G is optionally substituted heterocyclyl, optionally substituted heteroaryl, —N(R¹³)₂, —OR¹³, —CN, —OCON(R¹³)₂, or —N(R¹⁴)CON(R¹³)₂;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
W is a bond, —S—, —SO—, —SO₂—, —O—, —CR⁴R³—, —C≡C—C≡C—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR⁴R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the X groups.

One embodiment provides a compound, or a pharmaceutically acceptable salt thereof, having the structure of Formula (VI):

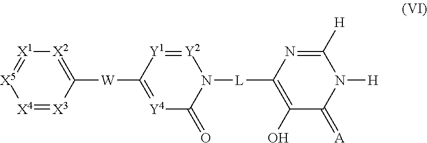

wherein:
A is O or S;
L is *—CR¹R²—CR³R⁴—; *—N(R⁵)—CR³R⁴—; *—O—CR³R⁴—; *—S—CR³R⁴—; *—N(R⁵)—C(O)—; *—C(O)—N(R⁵)—; *—N(R⁵)—S(O)₂—; *—S(O)₂—N(R⁵)—; or

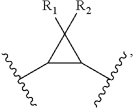

wherein the * denotes a bond to the hydroxypyrimidin-4(3H)-one ring or hydroxypyrimidin-4(3H)-thione ring;
R¹ or R³ are each independently H, optionally alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₁-C₄ alkylene)N(R¹²)—SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
each R⁵ is independently H or optionally substituted alkyl;
each X¹, X², X³, or X⁴ is N, N⁺—O⁻, or C—R⁶;
X⁵ is N, N⁺—O⁻, or C—R⁷;
each R⁶ is independently H, halogen, —OH, or optionally substituted C₁-C₃ alkyl; each Y¹, Y², Y³, or Y⁴ is N or C—R⁷;
R⁷ is halogen, —OH, or optionally substituted C₁-C₃ alkyl;
Z is H, -L1-G, optionally substituted alkyl, optionally substituted carbocyclyl, optionally substituted aryl, optionally substituted heteroaryl, optionally substituted heterocyclyl;
L1 is a bond, optionally substituted C₁-C₄ alkylene, —C(O)—, or —SO₂—;
G is optionally substituted heterocyclyl, optionally substituted heteroaryl, —N(R¹³)₂, —OR¹³, —CN, —OCON(R¹³)₂, or —N(R¹⁴)CON(R¹³)₂;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
W is a bond, —S—, —SO—, —SO₂—, —O—, —CR³R³—, —C≡C—C≡C—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the X groups.

Some embodiments provided herein describe a compound, or a pharmaceutically acceptable salt thereof, having the structure of Formula (VII):

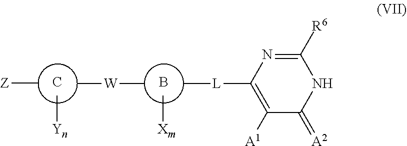

wherein:
n is 0, 1, or 2;
m is 0, 1, or 2;
A¹ is OH or SH;
A² is O or S;
L is a bond, —CR¹R², —N(R⁵)—, —O—, —S—, —SO₂—, *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR⁴R²—S—; *—N(R⁵)—C(O)—; *—C(O)—N(R⁵)—; *—N(R⁵)—S(O)₂—; *—S(O)₂—N(R⁵)—; or

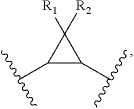

wherein the * denotes a bond to the pyrimidin-4(3H)-one ring or pyrimidin-4(3H)-thione ring;
R¹, R², R³, R⁴, and R⁵ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₀-C₄ alkylene)-CN, optionally substituted (C₀-C₄ alkylene)-OR¹¹, optionally substituted (C₀-C₄ alkylene)-N(R¹¹)₂, optionally substituted (C₀-C₄ alkylene)N(R¹²)—COR¹¹, optionally substituted (C₀-C₄ alkylene)N(R¹²)—CO₂R¹¹, optionally substituted (C₀-C₄ alkylene)N(R¹²)—CON(R¹¹)₂, optionally substituted (C₀-C₄ alkylene)N(R¹²)—SO₂N(R¹¹)₂, optionally substituted (C₀-C₄ alkylene)-O—SO₂N(R¹¹)₂, optionally substituted (C₀-C₄ alkylene)-N(R¹²)—SO₂R¹¹, optionally substituted (C₀-C₄ alkylene)-SO₂R¹¹, or optionally substituted (C₀-C₄ alkylene)N(R¹¹)—PO (optionally substituted C₀-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
R⁶ is H, halogen, optionally substituted alkyl, hydroxyl, alkoxyl, cyano, amino, or nitro;

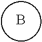

is phenyl,

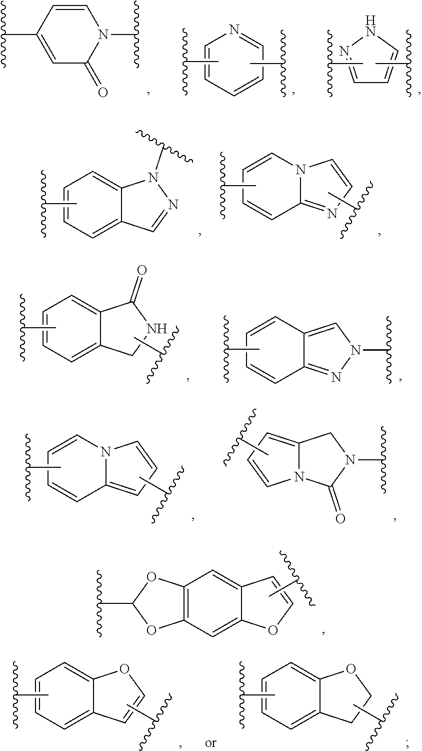

W is a bond, —C≡C—, —C≡C—C≡C—, —S—, —SO—, —SO₂—, —O—, —CR¹R³—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the Y groups;

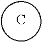

is absent, phenyl, alkyl, cycloalkyl,

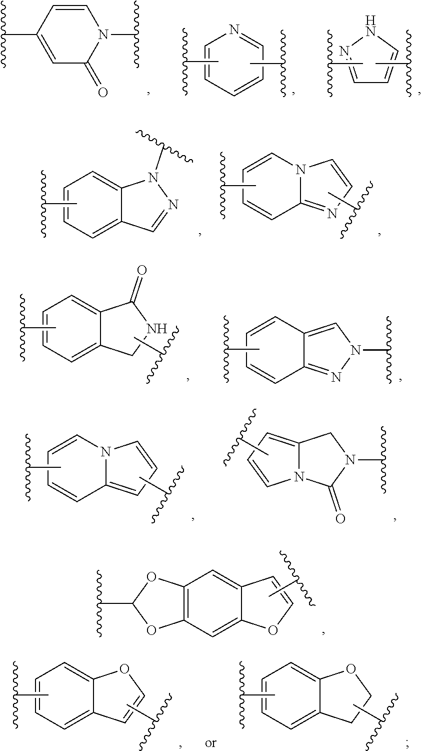

provided that if

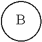

is phenyl or pyridine, then

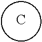

is not phenyl or pyridine; and
if

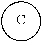

is phenyl or pyridine, then

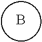

is not phenyl or pyridine;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L2-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L2 is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

Also provided herein in some embodiments is a compound, or a pharmaceutically acceptable salt thereof, having the structure of Formula (VII-1):

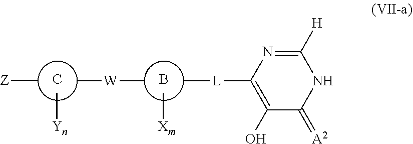

wherein:
n is 0, 1, or 2;
m is 0, 1, or 2;
A² is O or S;
L is —CR¹R²—, —N(R⁵)—, —O—, —S—, —SO₂—, *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR¹R²—S—; *—N(R⁵)—C(O)—; *—C(O)—N(R⁵)—; *—N(R⁵)—S(O)₂—; *—S(O)₂—N(R⁵)—; or

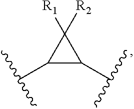

wherein the * denotes a bond to the hydroxypyrimidin-4(3H)-one ring or hydroxypyrimidin-4(3H)-thione ring;
R¹ or R³ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₀-C₄ alkylene)-CN, optionally substituted (C₀-C₄ alkylene)-OR¹¹, optionally substituted (C₀-C₄ alkylene)-N(R¹¹)₂, optionally substituted (C₀-C₄ alkylene)N(R¹²)—COR¹¹, optionally substituted (C₀-C₄ alkylene)N(R¹²)—CO₂R¹¹, optionally substituted (C₀-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₀-C₄ alkylene)N(R¹²)—SO₂N(R¹¹)₂, optionally substituted (C₀-C₄ alkylene)N(R¹²)—SO₂R¹¹, optionally substituted (C₀-C₄ alkylene)-O—SO₂N(R¹¹)₂, or optionally substituted (C₀-C₄ alkylene)N(R¹¹)—PO (optionally substituted C₁-C₄ alkyl)₂;
R², R⁴, and R⁵ are each independently H or optionally substituted C₁-C₃ alkyl;

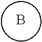

is phenyl,

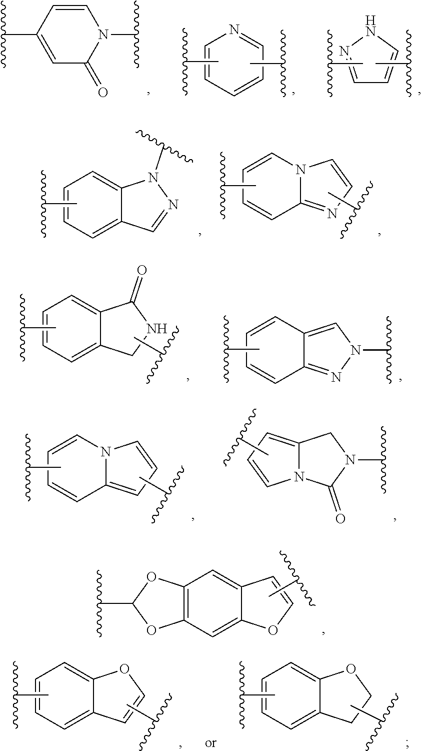

W is a bond, —C≡C—, —C≡C—C≡C—, —S—, —SO—, —SO₂—, —O—, —CR¹R³—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR⁴R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the Y groups:

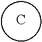

is absent, phenyl, alkyl, cycloalkyl,

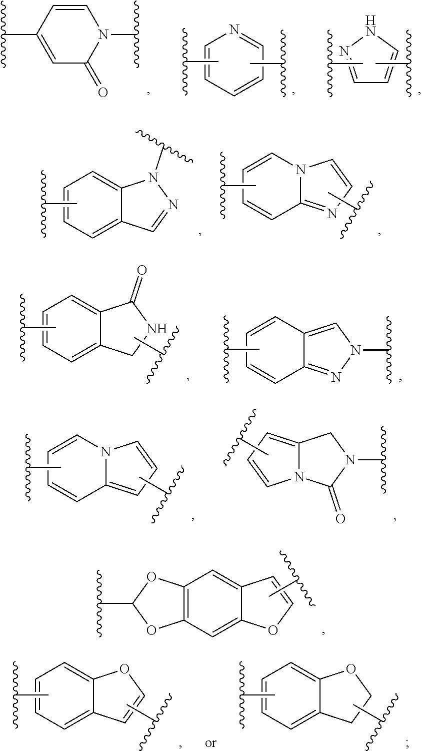

provided that if

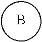

is phenyl or pyridine, then

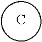

is not phenyl or pyridine; and
if

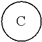

is phenyl or pyridine, then

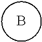

is not phenyl or pyridine;
X is halogen or optionally substituted C₁-C₃ alkyl;
Y is halogen or optionally substituted C₁-C₃ alkyl;
Z is H, optionally substituted alkyl, -L2-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂;
L2 is a bond, optionally substituted C₁-C₄ alkylene, —C(O)—, or —SO₂—;
G is optionally substituted heterocyclyl, optionally substituted heteroaryl, —N(R¹³)₂, —OR¹³, halogen, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

One embodiment provides a pharmaceutical composition comprising a compound disclosed herein or a pharmaceutically acceptable salt thereof, and a pharmaceutically acceptable excipient.

One embodiment provides for a method of treating a gram-negative bacterial infection in a patient in need thereof comprising administering to the patient a pharmaceutical composition comprising a compound disclosed herein or a pharmaceutically acceptable salt thereof, and a pharmaceutically acceptable excipient. One embodiment provides for a method wherein the gram-negative bacterial infection is selected from pneumonia, sepsis, cystic fibrosis, intra-abdominal infection, skin infection, and urinary tract infection. One embodiment provides for a method wherein the gram-negative bacterial infection is selected from chronic urinary tract infection, complicated urinary tract infection, cystitis, pyelonephritis, urethritis, recurrent urinary tract infections, bladder infections, urethral infections, and kidney infections.

One embodiment provides a method of inhibiting UDP-{3-O—[(R)-3-hydroxymyristoyl]}-N-acetylglucosamine deacetylase enzyme comprising contacting the enzyme with a compound disclosed herein.

One embodiment provides a method for treating bacterial infection in a patient in need thereof comprising administering to the patient a composition comprising a compound disclosed herein or a pharmaceutically acceptable salt thereof, and a pharmaceutically acceptable excipient.

### INCORPORATION BY REFERENCE

All publications, patents, and patent applications mentioned in this specification are herein incorporated by reference for the specific purposes identified herein.

### DETAILED DESCRIPTION OF THE INVENTION

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Aliphatic chain” refers to a linear chemical moiety that is composed of only carbons and hydrogens. In some embodiments, the aliphatic chain is saturated. In some embodiments, the aliphatic chain is unsaturated. In some embodiments, the unsaturated aliphatic chain contains one unsaturation. In some embodiments, the unsaturated aliphatic chain contains more than one unsaturation. In some embodiments, the unsaturated aliphatic chain contains two unsaturations. In some embodiments, the unsaturated aliphatic chain contains one double bond. In some embodiments, the unsaturated aliphatic chain contains two double bonds.

“Alkyl” refers to an optionally substituted straight-chain, or optionally substituted branched-chain saturated hydrocarbon monoradical having from one to about ten carbon atoms, or from one to six carbon atoms, wherein a sp3-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein a sp2-hybridized carbon of the alkenyl residue is attached to the rest of the molecule by a single bond. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5-to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuterium atoms. In some embodiments, the alkyl is substituted with one deuterium atom. In some embodiments, the alkyl is substituted with one, two, or three deuterium atoms. In some embodiments, the alkyl is substituted with one, two, three, four, five, or six deuterium atoms. Deuteroalkyl includes, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogen atoms. In some embodiments, the alkyl is substituted with one, two, or three halogen atoms. In some embodiments, the alkyl is substituted with one, two, three, four, five, or six halogen halogens.

Haloalkyl includes, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl.

“Halo” or “halogen” refers to bromo, chloro, fluoro or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized.

Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3-to 6-membered heterocycloalkyl. In some embodiments, the cycloalkyl is a 5-to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g. —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl. Unless stated otherwise specifically in the specification, a Heteroalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroaryl” refers to a 5-to 14-membered ring system radical comprising hydrogen atoms, one to thirteen carbon atoms, one to six heteroatoms selected from the group consisting of nitrogen, oxygen, phosphorous and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5-to 10-membered heteroaryl. In some embodiments, the heteroaryl is a 5-to 6-membered heteroaryl.

Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### LpxC Inhibitory Compounds

Provided herein are heterocyclic LpxC inhibitory compounds and pharmaceutical compositions comprising said compounds. The subject compounds and compositions are useful for inhibiting UDP-{3-O—[(R)-3-hydroxymyristoyl]}-N-acetylglucosamine deacetylase (LpxC) and for the treatment of bacterial infection.

One embodiment provides a compound, or a pharmaceutically acceptable salt thereof, having the structure of Formula (I):

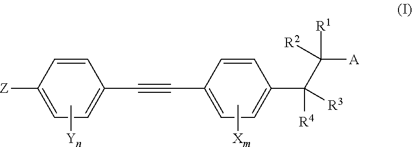

wherein:
A is

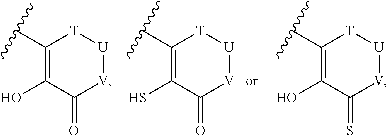

wherein the *-T-U—V— element is selected from the following:
a. *—C(R²¹)═C(R²¹)—N(R²²)—;
b. *—C(R²¹)═C(R²¹)—O—;
c. *—N(R²²)—C(R²¹)═C(R²¹)—;
d. *—N═C(R²¹)—N(R²³)—;
e. *—N═C(R²¹)—O—;
f. *—N(R²²)—N═C(R²¹)—;
g. *—C(R²¹)═N—N(R²²)—;
h. *—C(R²¹)═N—O—; and
i. *—O—C(R²¹)═C(R²¹)—;
each R²¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R²¹ groups together with the carbon atoms to which they are attached join to form a ring;
each R²² is independently H or optionally substituted alkyl; or a R²¹ group and a R²² group on adjacent atoms join to form a ring;
R²³ is optionally substituted alkyl;
n is 0-4;
m is 0-4;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₄-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
   provided that when A is

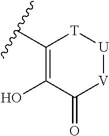

and *-T-U—V is *—O—C(R²¹)═C(R²¹)—, R³ is not H.

Another embodiment provides the compound of Formula (I), or a pharmaceutically acceptable salt thereof, wherein the *-T-U—V— element is *—C(R²¹)═C(R²¹)—N(R²²)—.

Another embodiment provides the compound of Formula (I), or a pharmaceutically acceptable salt thereof, wherein the *-T-U—V— element is *—C(R²¹)═C(R²¹)—O—.

Another embodiment provides the compound of Formula (I), or a pharmaceutically acceptable salt thereof, wherein the *-T-U—V— element is *—N(R²²)—C(R²¹)═C(R²¹)—.

Another embodiment provides the compound of Formula (I), or a pharmaceutically acceptable salt thereof, wherein the *-T-U—V— element is *—N═C(R²¹)—N(R²³)—.

Another embodiment provides the compound of Formula (I), or a pharmaceutically acceptable salt thereof, wherein the *-T-U—V— element is *—N═C(R²¹)—O—.

Another embodiment provides the compound of Formula (I), or a pharmaceutically acceptable salt thereof, wherein the *-T-U—V— element is *—N(R²²)—N═C(R²¹)—.

Another embodiment provides the compound of Formula (I), or a pharmaceutically acceptable salt thereof, wherein the *-T-U—V— element is *—C(R²¹)═N—N(R²²)—.

Another embodiment provides the compound of Formula (I), or a pharmaceutically acceptable salt thereof, wherein the *-T-U—V— element is *—C(R²¹)═N—O—.

Another embodiment provides the compound of Formula (I), or a pharmaceutically acceptable salt thereof, wherein the *-T-U—V— element is *—O—C(R²¹)═C(R²¹)—.

Another embodiment provides the compound of Formula (I), or a pharmaceutically acceptable salt thereof, wherein A is:

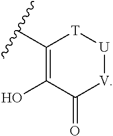

Another embodiment provides the compound of Formula (I), or a pharmaceutically acceptable salt thereof, wherein A is:

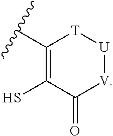

Another embodiment provides the compound of Formula (I), or a pharmaceutically acceptable salt thereof, wherein A is:

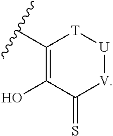

One embodiment provides a compound, or a pharmaceutically acceptable salt thereof, having the structure of Formula (II):

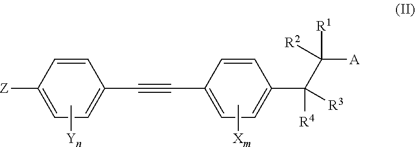

wherein:
A is

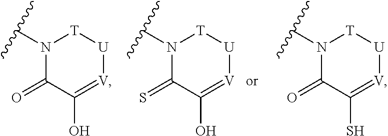

wherein the *-T-U—V— element is selected from the following:
a. *—C(R²¹)═C(R²¹)—C(R²¹)═;
b. *—N═C(R²¹)—C(R²¹)═;
c. *—C(R²¹)═N—C(R²¹)═;
d. *—C(R²¹)═C(R²¹)—N═;
e. *—N═C(R²¹)—N═;
f. *—C(R²¹)═N—N═;
g. *—N═N—C(R²¹)═; and
h. *—N═N—N═;
each R²¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R²¹ groups together with the carbon atoms to which they are attached join to form a ring;
n is 0-4;
m is 0-4;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₄-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

Another embodiment provides the compound of Formula (II), or a pharmaceutically acceptable salt thereof, wherein the *-T-U—V— element is *—C(R²¹)═C(R²¹)—C(R²¹)═.

Another embodiment provides the compound of Formula (II), or a pharmaceutically acceptable salt thereof, wherein the *-T-U—V— element is *—N═C(R²¹)—C(R²¹)═.

Another embodiment provides the compound of Formula (II), or a pharmaceutically acceptable salt thereof, wherein the *-T-U—V— element is *—C(R²¹)═N—C(R²¹)═.

Another embodiment provides the compound of Formula (II), or a pharmaceutically acceptable salt thereof, wherein the *-T-U—V— element is *—C(R²¹)═C(R²¹)—N═.

Another embodiment provides the compound of Formula (II), or a pharmaceutically acceptable salt thereof, wherein the *-T-U—V— element is *—N═C(R²¹)—N═.

Another embodiment provides the compound of Formula (II), or a pharmaceutically acceptable salt thereof, wherein the *-T-U—V— element is *—C(R²¹)═N—N═.

Another embodiment provides the compound of Formula (II), or a pharmaceutically acceptable salt thereof, wherein the *-T-U—V— element is *—N═N—C(R²¹)═.

Another embodiment provides the compound of Formula (II), or a pharmaceutically acceptable salt thereof, wherein the *-T-U—V— element is *—N═N—N═.

Another embodiment provides the compound of Formula (II), or a pharmaceutically acceptable salt thereof, wherein A is:

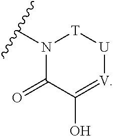

Another embodiment provides the compound of Formula (II), or a pharmaceutically acceptable salt thereof, wherein A is:

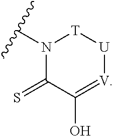

Another embodiment provides the compound of Formula (II), or a pharmaceutically acceptable salt thereof, wherein A is:

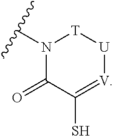

One embodiment provides a compound, or a pharmaceutically acceptable salt thereof, having the structure of Formula (III):

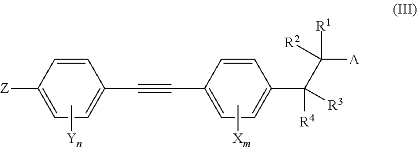

wherein:
A is

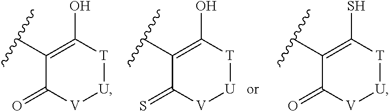

wherein the *-T-U—V— element is selected from the following:
a. *—C(R²¹)═C(R²¹)—N(R²²)—;
b. *—C(R²¹)═C(R²¹)—O—;
c. *—N(R²²)—C(R²¹)═C(R²¹)—;
d. *—N═C(R²¹)—N(R²²)—;
e. *—N═C(R²¹)—O—;
f. *—N(R²²)—N═C(R²¹)—;
g. *—C(R²¹)═N—N(R²²)—; and
h. *—C(R²¹)═N—O—;
each R²¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R²¹ groups together with the carbon atoms to which they are attached join to form a ring;
each R²² is independently H or optionally substituted alkyl; or a R²¹ group and a R²² group on adjacent atoms join to form a ring;
n is 0-4;
m is 0-4;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₄-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

Another embodiment provides the compound of Formula (III), or a pharmaceutically acceptable salt thereof, wherein the *-T-U—V— element is *—C(R²¹)═C(R²¹)—N(R²²)—.

Another embodiment provides the compound of Formula (III), or a pharmaceutically acceptable salt thereof, wherein the *-T-U—V— element is *—C(R²¹)═C(R²¹)—O—.

Another embodiment provides the compound of Formula (III), or a pharmaceutically acceptable salt thereof, wherein the *-T-U—V— element is *—N(R²²)—C(R²¹)═C(R²¹)—.

Another embodiment provides the compound of Formula (III), or a pharmaceutically acceptable salt thereof, wherein the *-T-U—V— element is *—N═C(R²¹)—N(R²²)—.

Another embodiment provides the compound of Formula (III), or a pharmaceutically acceptable salt thereof, wherein the *-T-U—V— element is *—N═C(R²¹)—O—.

Another embodiment provides the compound of Formula (III), or a pharmaceutically acceptable salt thereof, wherein the *-T-U—V— element is *—N(R²²)—N═C(R²¹)—.

Another embodiment provides the compound of Formula (III), or a pharmaceutically acceptable salt thereof, wherein the *-T-U—V— element is *—C(R²¹)═N—N(R²²)—.

Another embodiment provides the compound of Formula (III), or a pharmaceutically acceptable salt thereof, wherein the *-T-U—V— element is *—C(R²¹)═N—O—.

Another embodiment provides the compound of Formula (III), or a pharmaceutically acceptable salt thereof, wherein A is:

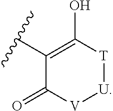

Another embodiment provides the compound of Formula (III), or a pharmaceutically acceptable salt thereof, wherein A is:

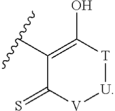

Another embodiment provides the compound of Formula (III), or a pharmaceutically acceptable salt thereof, wherein A is:

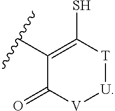

Another embodiment provides the compound of Formula (I), (II), or (III), or a pharmaceutically acceptable salt thereof, wherein each R²¹ is H.

Another embodiment provides the compound of Formula (I), (II), or (III), or a pharmaceutically acceptable salt thereof, wherein each R²¹ is independently H or optionally substituted alkyl. Another embodiment provides the compound of Formula (I), (II), or (III), or a pharmaceutically acceptable salt thereof, wherein each R²¹ is independently H or optionally substituted alkenyl. Another embodiment provides the compound of Formula (I), (II), or (III), or a pharmaceutically acceptable salt thereof, wherein each R²¹ is independently H or optionally substituted carbocyclyl. Another embodiment provides the compound of Formula (I), (II), or (III), or a pharmaceutically acceptable salt thereof, wherein each R²¹ is independently H or optionally substituted carbocyclylalkyl. Another embodiment provides the compound of Formula (I), (II), or (III), or a pharmaceutically acceptable salt thereof, wherein each R²¹ is independently H or optionally substituted aryl. Another embodiment provides the compound of Formula (I), (II), or (III), or a pharmaceutically acceptable salt thereof, wherein each R²¹ is independently H or optionally substituted aralkyl. Another embodiment provides the compound of Formula (I), (II), or (III), or a pharmaceutically acceptable salt thereof, wherein each R²¹ is independently H or optionally substituted heterocyclyl. Another embodiment provides the compound of Formula (I), (II), or (III), or a pharmaceutically acceptable salt thereof, wherein each R²¹ is independently H or optionally substituted heterocyclylalkyl. Another embodiment provides the compound of Formula (I), (II), or (III), or a pharmaceutically acceptable salt thereof, wherein each R²¹ is independently H or optionally substituted heteroaryl. Another embodiment provides the compound of Formula (I), (II), or (III), or a pharmaceutically acceptable salt thereof, wherein each R²¹ is independently H or optionally substituted heteroarylalkyl. Another embodiment provides the compound of Formula (I), (II), or (III), or a pharmaceutically acceptable salt thereof, wherein two R²¹ groups together with the carbon atoms to which they are attached join to form a ring.

Another embodiment provides the compound of Formula (I), (II), or (III), or a pharmaceutically acceptable salt thereof, wherein R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂.

Another embodiment provides the compound of Formula (I), (II), or (III), or a pharmaceutically acceptable salt thereof, wherein R¹ is unsubstituted alkyl, —COR¹¹, —CON(R¹¹)₂, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, or optionally substituted (C₁-C₄ alkylene)N(R¹²)—CON(R¹¹)₂. Another embodiment provides the compound of Formula (I), (II), or (III), or a pharmaceutically acceptable salt thereof, wherein R¹ is —COR¹¹, —CON(R¹¹)₂, optionally substituted (C₁-C₄ alkylene)-N(R¹¹)₂, or optionally substituted (C₁-C₄ alkylene)N(R¹²)—COR¹¹. Another embodiment provides the compound of Formula (I), (II), or (III), or a pharmaceutically acceptable salt thereof, wherein R¹ is —COR¹¹. Another embodiment provides the compound of Formula (I), (II), or (III), or a pharmaceutically acceptable salt thereof, wherein R¹ is —CON(R¹¹)₂. Another embodiment provides the compound of Formula (I), (II), or (III), or a pharmaceutically acceptable salt thereof, wherein R¹ is optionally substituted (C₁-C₄ alkylene)-N(R¹¹)₂. Another embodiment provides the compound of Formula (I), (II), or (III), or a pharmaceutically acceptable salt thereof, wherein R¹ is optionally substituted (C₁-C₄ alkylene)N(R¹²)—COR¹¹. Another embodiment provides the compound of Formula (I), (II), or (III), or a pharmaceutically acceptable salt thereof, wherein optionally substituted (C₁-C₄ alkylene) is —CH₂—.

Another embodiment provides the compound of Formula (I), (II), or (III), or a pharmaceutically acceptable salt thereof, wherein each R¹¹ is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl. Another embodiment provides the compound of Formula (I), (II), or (III), or a pharmaceutically acceptable salt thereof, wherein R¹¹ is H. Another embodiment provides the compound of Formula (I), (II), or (III), or a pharmaceutically acceptable salt thereof, wherein R¹¹ is optionally substituted alkyl. Another embodiment provides the compound of Formula (I), (II), or (III), or a pharmaceutically acceptable salt thereof, wherein R¹¹ is hydroxy substituted alkyl. Another embodiment provides the compound of Formula (I), (II), or (III), or a pharmaceutically acceptable salt thereof, wherein R¹¹ is fluoro substituted alkyl. Another embodiment provides the compound of Formula (I), (II), or (III), or a pharmaceutically acceptable salt thereof, wherein R¹¹ is 2-fluoroethyl. Another embodiment provides the compound of Formula (I), (II), or (III), or a pharmaceutically acceptable salt thereof, wherein R¹¹ is fluoromethyl.

Another embodiment provides the compound of Formula (I), (II), or (III), or a pharmaceutically acceptable salt thereof, wherein R¹¹ is difluoro substituted alkyl. Another embodiment provides the compound of Formula (I), (II), or (III), or a pharmaceutically acceptable salt thereof, wherein R¹¹ is 2,2-difluoroethyl. Another embodiment provides the compound of Formula (I), (II), or (III), or a pharmaceutically acceptable salt thereof, wherein R¹¹ is difluoromethyl. Another embodiment provides the compound of Formula (I), (II), or (III), or a pharmaceutically acceptable salt thereof, wherein R¹¹ is trifluoro substituted alkyl. Another embodiment provides the compound of Formula (I), (II), or (III), or a pharmaceutically acceptable salt thereof, wherein R¹¹ is 2,2,2-trifluoroethyl. Another embodiment provides the compound of Formula (I), (II), or (III), or a pharmaceutically acceptable salt thereof, wherein R¹¹ is trifluoromethyl. Another embodiment provides the compound of Formula (I), (II), or (III), or a pharmaceutically acceptable salt thereof, wherein R¹¹ is amino substituted alkyl. Another embodiment provides the compound of Formula (I), (II), or (III), or a pharmaceutically acceptable salt thereof, wherein R¹¹ is cyano substituted alkyl. Another embodiment provides the compound of Formula (I), (II), or (III), or a pharmaceutically acceptable salt thereof, wherein R¹¹ is optionally substituted alkenyl. Another embodiment provides the compound of Formula (I), (II), or (III), or a pharmaceutically acceptable salt thereof, wherein R¹¹ is optionally substituted carbocyclyl. Another embodiment provides the compound of Formula (I), (II), or (III), or a pharmaceutically acceptable salt thereof, wherein R¹¹ is optionally substituted carbocyclylalkyl. Another embodiment provides the compound of Formula (I), (II), or (III), or a pharmaceutically acceptable salt thereof, wherein R¹¹ is optionally substituted heterocyclyl.

Another embodiment provides the compound of Formula (I), (II), or (III), or a pharmaceutically acceptable salt thereof, wherein R¹¹ is optionally substituted heterocyclylalkyl. Another embodiment provides the compound of Formula (I), (II), or (III), or a pharmaceutically acceptable salt thereof, wherein R¹¹ is optionally substituted heteroaryl. Another embodiment provides the compound of Formula (I), (II), or (III), or a pharmaceutically acceptable salt thereof, wherein R¹¹ is optionally substituted heteroarylalkyl. Another embodiment provides the compound of Formula (I), (II), or (III), or a pharmaceutically acceptable salt thereof, wherein two R¹¹ groups together with the nitrogen to which they are attached join to form an optionally substituted N-heterocyclyl. Another embodiment provides the compound of Formula (I), (II), or (III), or a pharmaceutically acceptable salt thereof, wherein R¹¹ is vinyl, propan-2-yl, methyl, ethyl, cyclopropyl, cyclopentyl, azentidin-1-yl or allyl.

Another embodiment provides the compound of Formula (I), (II), or (III), or a pharmaceutically acceptable salt thereof, wherein R² is H.

Another embodiment provides the compound of Formula (I), (II), or (III), or a pharmaceutically acceptable salt thereof, wherein R³ is H.

Another embodiment provides the compound of Formula (I), (II), or (III), or a pharmaceutically acceptable salt thereof, wherein R⁴ is H.

Another embodiment provides the compound of Formula (I), (II), or (III), or a pharmaceutically acceptable salt thereof, wherein n is 0 or 1. Another embodiment provides the compound of Formula (I), (II), or (III), or a pharmaceutically acceptable salt thereof, wherein n is 1 or 2.

Another embodiment provides the compound of Formula (I), (II), or (III), or a pharmaceutically acceptable salt thereof, wherein m is 0 or 1. Another embodiment provides the compound of Formula (I), (II), or (III), or a pharmaceutically acceptable salt thereof, wherein m is 1 or 2.

Another embodiment provides the compound of Formula (I), (II), or (III), or a pharmaceutically acceptable salt thereof, wherein X is halogen.

Another embodiment provides the compound of Formula (I), (II), or (III), or a pharmaceutically acceptable salt thereof, wherein Y is halogen.

Another embodiment provides the compound of Formula (I), (II), or (III), or a pharmaceutically acceptable salt thereof, wherein Z is -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂. Another embodiment provides the compound of Formula (I), (II), or (III), or a pharmaceutically acceptable salt thereof, wherein Z is -L-G. Another embodiment provides the compound of Formula (I), (II), or (III), or a pharmaceutically acceptable salt thereof, wherein Z is morpholinomethyl, (8-oxa-3-azabicyclo[3.2.1]octan-3-yl) methyl, (2-oxa-5-azabicyclo[2.2.1]heptan-5-yl) methyl, (1,1-dioxidothiomorpholino) methyl, (oxetan-3-ylamino) methyl, ((methoxycyclobutyl)amino)methyl, (methylpiperazin-1-yl)methyl, (cyanopyrrolidin-1-yl) methyl, or (methoxypyrrolidin-1-yl) methyl.

Another embodiment provides the compound of Formula (I), (II), or (III), or a pharmaceutically acceptable salt thereof, wherein L is a bond or optionally substituted C₁-C₄ alkylene.

Another embodiment provides the compound of Formula (I), (II), or (III), or a pharmaceutically acceptable salt thereof, wherein L is a bond. Another embodiment provides the compound of Formula (I), (II), or (III), or a pharmaceutically acceptable salt thereof, wherein L is an optionally substituted C₁-C₄ alkylene. Another embodiment provides the compound of Formula (I), (II), or (III), or a pharmaceutically acceptable salt thereof, wherein L is optionally substituted C₁. Another embodiment provides the compound of Formula (I), (II), or (III), or a pharmaceutically acceptable salt thereof, wherein L is —CH₂—.

Another embodiment provides the compound of Formula (I), (II), or (III), or a pharmaceutically acceptable salt thereof, wherein G is optionally substituted alkyl. Another embodiment provides the compound of Formula (I), (II), or (III), or a pharmaceutically acceptable salt thereof, wherein G is optionally substituted heterocyclyl. Another embodiment provides the compound of Formula (I), (II), or (III), or a pharmaceutically acceptable salt thereof, wherein G is optionally substituted morpholinyl, piperidinyl, piperazinyl, pyrolidinyl, imidazolyl, imidazolidinyl. Another embodiment provides the compound of Formula (I), (II), or (III), or a pharmaceutically acceptable salt thereof, wherein G is optionally substituted morpholinyl. Another embodiment provides the compound of Formula (I), (II), or (III), or a pharmaceutically acceptable salt thereof, wherein G is unsubstituted morpholinyl Another embodiment provides the compound of Formula (I), (II), or (III), or a pharmaceutically acceptable salt thereof, wherein G is optionally substituted heteroaryl. Another embodiment provides the compound of Formula (I), (II), or (III), or a pharmaceutically acceptable salt thereof, wherein G is —N(R¹³)₂. Another embodiment provides the compound of Formula (I), (II), or (III), or a pharmaceutically acceptable salt thereof, wherein G is —OR¹³. Another embodiment provides the compound of Formula (I), (II), or (III), or a pharmaceutically acceptable salt thereof, wherein G is —CN. Another embodiment provides the compound of Formula (I), (II), or (III), or a pharmaceutically acceptable salt thereof, wherein each R¹³ is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl. Another embodiment provides the compound of Formula (I), (II), or (III), or a pharmaceutically acceptable salt thereof, wherein R¹³ is H. Another embodiment provides the compound of Formula (I), (II), or (III), or a pharmaceutically acceptable salt thereof, wherein R¹³ is optionally substituted alkyl. Another embodiment provides the compound of Formula (I), (II), or (III), or a pharmaceutically acceptable salt thereof, wherein R¹³ is optionally substituted carbocyclyl. Another embodiment provides the compound of Formula (I), (II), or (III), or a pharmaceutically acceptable salt thereof, wherein R¹³ is optionally substituted carbocyclylalkyl. Another embodiment provides the compound of Formula (I), (II), or (III), or a pharmaceutically acceptable salt thereof, wherein R¹³ is optionally substituted heterocyclyl. Another embodiment provides the compound of Formula (I), (II), or (III), or a pharmaceutically acceptable salt thereof, wherein R¹³ is optionally substituted heterocyclylalkyl. Another embodiment provides the compound of Formula (I), (II), or (III), or a pharmaceutically acceptable salt thereof, wherein R¹³ is optionally substituted heteroaryl. Another embodiment provides the compound of Formula (I), (II), or (III), or a pharmaceutically acceptable salt thereof, wherein R¹³ is optionally substituted heteroarylalkyl. Another embodiment provides the compound of Formula (I), (II), or (III), or a pharmaceutically acceptable salt thereof, wherein two R¹³ groups together with the nitrogen to which they are attached join to form an optionally substituted N-heterocyclyl.

One embodiment provides for a compound, or a pharmaceutically acceptable salt thereof, of Formula (IV) wherein the structure is:

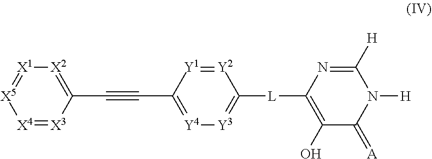

wherein.
A is O or S;
L is *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR¹R²—S—; *—N(R³)—C(O)—; *—C(O)—N(R⁵)—; *—N(R⁵)—S(O)₂—; *—S(O)₂—N(R⁵)—; or

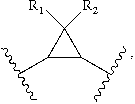

wherein the * denotes a bond to the hydroxypyrimidin-4(3H)-one ring or hydroxypyrimidin-4(3H)-thione ring;
R¹ or R³ are each independently H, optionally alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₁-C₄ alkylene)N(R¹²)—SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
each R⁵ is independently H or optionally substituted alkyl;
each X¹, X², X³, or X⁴ is N, N⁺—O⁻, or C—R⁶;
X⁵ is N, N⁺—O⁻, or C—R⁷;
each R⁶ is independently H, halogen, —OH, or optionally substituted C₁-C₃ alkyl; each Y¹, Y², Y³, or Y⁴ is N, N⁺—O⁻, or C—R⁷;
R⁷ is halogen, —OH, or optionally substituted C₁-C₃ alkyl;
Z is H, -L1-G, optionally substituted alkyl, optionally substituted carbocyclyl, optionally substituted aryl, optionally substituted heteroaryl, optionally substituted heterocyclyl;
L1 is a bond, optionally substituted C₁-C₄ alkylene, —C(O)—, or —SO₂—;
G is optionally substituted heterocyclyl, optionally substituted heteroaryl, —N(R¹³)₂, —OR¹³, —CN, —OCON(R¹³)₂, or —N(R¹⁴)CON(R¹³)₂;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
with the provision if L is *—CR¹R²—CR³R⁴—, then at least one of X¹, X², X³, X⁴, X⁵, Y¹, Y², Y³, or Y⁴ is not C.

One embodiment provides for a compound, or a pharmaceutically acceptable salt thereof, of Formula (IV) wherein A is O. One embodiment provides for a compound, or a pharmaceutically acceptable salt thereof, of Formula (IV) wherein L is *—CR¹R²—CR³R⁴—. One embodiment provides for a compound, or a pharmaceutically acceptable salt thereof, of Formula (IV) wherein L is *—N(R⁵)—CR³R⁴—. One embodiment provides for a compound, or a pharmaceutically acceptable salt thereof, of Formula (IV) wherein L is *—CR¹R²—N(R⁵)—. One embodiment provides for a compound, or a pharmaceutically acceptable salt thereof, of Formula (IV) wherein L is *—CR¹R²—O—. One embodiment provides for a compound, or a pharmaceutically acceptable salt thereof, of Formula (IV) wherein L is *—CR¹R²—S—. One embodiment provides for a compound, or a pharmaceutically acceptable salt thereof, of Formula (IV) wherein L is *—N(R⁵)—C(O)—. One embodiment provides for a compound, or a pharmaceutically acceptable salt thereof, of Formula (IV) wherein L is *—C(O)—N(R⁵)—. One embodiment provides fora compound, or a pharmaceutically acceptable salt thereof, of Formula (IV) wherein L is *—N(R⁵)—S(O)₂—. One embodiment provides for a compound, or a pharmaceutically acceptable salt thereof, of Formula (IV) wherein R¹ or R³ is —COR¹¹. One embodiment provides for a compound, or a pharmaceutically acceptable salt thereof, of Formula (IV) wherein R¹ or R³ is —CON(R¹¹)₂.

One embodiment provides for a compound, or a pharmaceutically acceptable salt thereof, of Formula (IV) wherein R¹ or R³ is optionally substituted (C₁-C₄ alkylene)-N(R¹¹)₂. One embodiment provides for a compound, or a pharmaceutically acceptable salt thereof, of Formula (IV) wherein R¹ or R³ is optionally substituted (C₁-C₄ alkylene)N(R¹²)—COR¹¹. One embodiment provides for a compound, or a pharmaceutically acceptable salt thereof, of Formula (IV) wherein R¹ or R³ is optionally substituted (C₁-C₄ alkylene)N(R¹²)—CO2R¹¹.

One embodiment provides for a compound, or a pharmaceutically acceptable salt thereof, of Formula (IV) wherein R² is H. One embodiment provides for a compound, or a pharmaceutically acceptable salt thereof, of Formula (IV) wherein R⁴ is H. One embodiment provides for a compound, or a pharmaceutically acceptable salt thereof, of Formula (IV) wherein R⁵ is H.

One embodiment provides for a compound, or a pharmaceutically acceptable salt thereof, of Formula (IV) wherein X¹, X² and X³ are CR⁶ and X⁴ is N. One embodiment provides for a compound, or a pharmaceutically acceptable salt thereof, of Formula (IV) wherein X¹, X² and X⁴ are CR⁶ and X³ is N. One embodiment provides for a compound, or a pharmaceutically acceptable salt thereof, of Formula (IV) wherein X¹, X³ and X⁴ are CR⁶ and X² is N. One embodiment provides for a compound, or a pharmaceutically acceptable salt thereof, of Formula (IV) wherein X², X³ and X⁴ are CR⁶ and X¹ is N. One embodiment provides for a compound, or a pharmaceutically acceptable salt thereof, of Formula (IV) wherein X¹ and X² is CR⁶, X³ and X⁴ is N. One embodiment provides for a compound, or a pharmaceutically acceptable salt thereof, of Formula (IV) wherein X¹ and X³ is CR⁶, X² and X⁴ is N. One embodiment provides for a compound, or a pharmaceutically acceptable salt thereof, of Formula (IV) wherein X¹ and X⁴ is CR⁶, X³ and X² is N. One embodiment provides for a compound, or a pharmaceutically acceptable salt thereof, of Formula (IV) wherein X² and X³ is CR⁶, X¹ and X⁴ is N. One embodiment provides for a compound, or a pharmaceutically acceptable salt thereof, of Formula (IV) wherein X² and X⁴ is CR⁶, X¹ and X³ is N. One embodiment provides for a compound, or a pharmaceutically acceptable salt thereof, of Formula (IV) wherein X³ and X⁴ is CR⁶, X¹ and X² is N.

One embodiment provides for a compound, or a pharmaceutically acceptable salt thereof, of Formula (IV) wherein X⁵ is C—Z. One embodiment provides for a compound, or a pharmaceutically acceptable salt thereof, of Formula (IV) wherein X⁵ is N. One embodiment provides for a compound, or a pharmaceutically acceptable salt thereof, of Formula (IV) wherein R⁶ is halogen. One embodiment provides for a compound, or a pharmaceutically acceptable salt thereof, of Formula (IV) wherein R⁶ is H. One embodiment provides for a compound, or a pharmaceutically acceptable salt thereof, of Formula (IV) wherein R⁶ is —OH.

One embodiment provides for a compound, or a pharmaceutically acceptable salt thereof, of Formula (IV) wherein Y¹, Y² and Y³ are CR⁷ and Y⁴ is N. One embodiment provides for a compound, or a pharmaceutically acceptable salt thereof, of Formula (IV) wherein Y¹, Y² and Y⁴ are CR⁷ and Y³ is N. One embodiment provides for a compound, or a pharmaceutically acceptable salt thereof, of Formula (IV) wherein Y¹, Y³ and Y⁴ are CR⁷ and Y² is N. One embodiment provides for a compound, or a pharmaceutically acceptable salt thereof, of Formula (IV) wherein Y², Y³ and Y⁴ are CR⁷ and Y¹ is N. One embodiment provides for a compound, or a pharmaceutically acceptable salt thereof, of Formula (IV) wherein Y¹ and Y² is CR⁷, Y³ and Y⁴ is N. One embodiment provides for a compound, or a pharmaceutically acceptable salt thereof, of Formula (IV) wherein Y¹ and Y³ is CR⁷, Y² and Y⁴ is N. One embodiment provides for a compound, or a pharmaceutically acceptable salt thereof, of Formula (IV) wherein Y¹ and Y⁴ is CR⁷, Y³ and Y² is N. One embodiment provides for a compound, or a pharmaceutically acceptable salt thereof, of Formula (IV) wherein Y² and Y³ is CR⁷, Y¹ and Y⁴ is N. One embodiment provides for a compound, or a pharmaceutically acceptable salt thereof, of Formula (IV) wherein Y² and Y⁴ is CR⁷, Y¹ and Y³ is N. One embodiment provides for a compound, or a pharmaceutically acceptable salt thereof, of Formula (IV) wherein Y³ and Y⁴ is CR⁷, Y¹ and Y² is N.

One embodiment provides for a compound, or a pharmaceutically acceptable salt thereof, of Formula (IV) wherein R⁷ is halogen. One embodiment provides for a compound, or a pharmaceutically acceptable salt thereof, of Formula (IV) wherein R⁷ is H. One embodiment provides for a compound, or a pharmaceutically acceptable salt thereof, of Formula (IV) wherein R⁷ is —OH.

One embodiment provides for a compound, or a pharmaceutically acceptable salt thereof, of Formula (IV) wherein Z is H. One embodiment provides for a compound, or a pharmaceutically acceptable salt thereof, of Formula (IV) wherein Z is haloalkyl. One embodiment provides for a compound, or a pharmaceutically acceptable salt thereof, of Formula (IV) wherein Z is hydroxyalkyl.

One embodiment provides for a compound, or a pharmaceutically acceptable salt thereof, of Formula (IV) wherein Z is -L1-G. One embodiment provides for a compound, or a pharmaceutically acceptable salt thereof, of Formula (IV) wherein L1 is a bond. One embodiment provides for a compound, or a pharmaceutically acceptable salt thereof, of Formula (IV) wherein L1 is a C₁-C₄ alkylene. One embodiment provides for a compound, or a pharmaceutically acceptable salt thereof, of Formula (IV) wherein L1 is C₁ alkylene. One embodiment provides for a compound, or a pharmaceutically acceptable salt thereof, of Formula (IV) wherein L1 is —CH₂—. One embodiment provides for a compound, or a pharmaceutically acceptable salt thereof, of Formula (IV) wherein G is —N(R¹³)₂—.

One embodiment provides for a compound, or a pharmaceutically acceptable salt thereof, of Formula (IV) wherein R¹³ is H. One embodiment provides for a compound, or a pharmaceutically acceptable salt thereof, of Formula (IV) wherein R¹³ is optionally substituted alkyl. One embodiment provides for a compound, or a pharmaceutically acceptable salt thereof, of Formula (IV) wherein R¹³ is hydroxy substituted alkyl. One embodiment provides for a compound, or a pharmaceutically acceptable salt thereof, of Formula (IV) wherein R¹³ is optionally substituted heterocycle. One embodiment provides for a compound, or a pharmaceutically acceptable salt thereof, of Formula (IV) wherein R¹³ is alkoxy substituted heterocycle. One embodiment provides for a compound, or a pharmaceutically acceptable salt thereof, of Formula (IV) wherein R¹³ is optionally substituted cycloalkyl.

One embodiment provides for a compound, or a pharmaceutically acceptable salt thereof, of Formula (V) wherein the structure is:

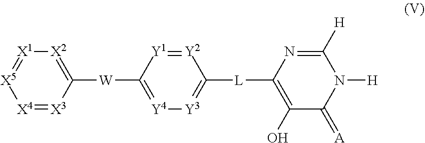

wherein:
A is O or S;
L is *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR¹R²—S—; *—N(R⁵)—C(O)—; *—C(O)—N(R⁵)—; *—N(R⁵)—S(O)₂—; *—S(O)₂—N(R⁵)—; or

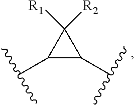

wherein the * denotes a bond to the hydroxypyrimidin-4(3H)-one ring or hydroxypyrimidin-4(3H)-thione ring;
R¹ or R³ are each independently H, optionally alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₁-C₄ alkylene)N(R¹²)—SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
each R⁵ is independently H or optionally substituted alkyl;
each X¹, X², X³, or X⁴ is N, N⁺—O⁻, or C—R⁶;
X⁵ is N, N⁺—O⁻, or C—Z;
each R⁶ is independently H, halogen, —OH, or optionally substituted C₁-C₃ alkyl; each Y¹, Y², Y³, or Y⁴ is N, N⁺—O⁻, or C—R⁷;
R⁷ is halogen, —OH, or optionally substituted C₁-C₃ alkyl;
Z is H, -L1-G, optionally substituted alkyl, optionally substituted carbocyclyl, optionally substituted aryl, optionally substituted heteroaryl, optionally substituted heterocyclyl;
L1 is a bond, optionally substituted C₁-C₄ alkylene, —C(O)—, or —SO₂—;
G is optionally substituted heterocyclyl, optionally substituted heteroaryl, —N(R¹³)₂, —OR¹³, —CN, —OCON(R¹³)₂, or —N(R¹⁴)CON(R¹³)₂;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
W is a bond, —S—, —SO—, —SO₂—, —O—, —CR¹R³—, —C≡C—C≡C—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR¹R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the X groups.

One embodiment provides for a compound, or a pharmaceutically acceptable salt thereof, of Formula (V) wherein A is O. One embodiment provides for a compound, or a pharmaceutically acceptable salt thereof, of Formula (V) wherein L is *—CR¹R²—CR³R⁴—. One embodiment provides for a compound, or a pharmaceutically acceptable salt thereof, of Formula (V) wherein L is *—N(R⁵)—CR³R⁴—. One embodiment provides for a compound, or a pharmaceutically acceptable salt thereof, of Formula (V) wherein L is *—CR¹R²—N(R⁵)—. One embodiment provides for a compound, or a pharmaceutically acceptable salt thereof, of Formula (V) wherein L is*—CR¹R²—O—. One embodiment provides for a compound, or a pharmaceutically acceptable salt thereof, of Formula (V) wherein L is *—CR¹R²—S—. One embodiment provides for a compound, or a pharmaceutically acceptable salt thereof, of Formula (V) wherein L is *—N(R⁵)—C(O)—. One embodiment provides for a compound, or a pharmaceutically acceptable salt thereof, of Formula (V) wherein L is *—C(O)—N(R⁵)—. One embodiment provides for a compound, or a pharmaceutically acceptable salt thereof, of Formula (V) wherein L is *—N(R⁵)—S(O)₂—. One embodiment provides for a compound, or a pharmaceutically acceptable salt thereof, of Formula (V) wherein

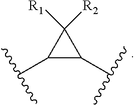

One embodiment provides for a compound, or a pharmaceutically acceptable salt thereof, of Formula (V) wherein R¹ or R³ is —COR¹¹. One embodiment provides for a compound, or a pharmaceutically acceptable salt thereof, of Formula (V) wherein R¹ or R³ is —CON(R¹¹)₂.

One embodiment provides for a compound, or a pharmaceutically acceptable salt thereof, of Formula (V) wherein R¹ or R³ is optionally substituted (C₁-C₄ alkylene)-N(R¹¹)₂. One embodiment provides for a compound, or a pharmaceutically acceptable salt thereof, of Formula (V) wherein R¹ or R³ is optionally substituted (C₁-C₄ alkylene)N(R¹²)—COR¹¹. One embodiment provides for a compound, or a pharmaceutically acceptable salt thereof, of Formula (V) wherein R¹ or R³ is optionally substituted (C₁-C₄ alkylene)N(R¹²)—CO₂R¹¹.

One embodiment provides for a compound, or a pharmaceutically acceptable salt thereof, of Formula (V) wherein R² is H. One embodiment provides for a compound, or a pharmaceutically acceptable salt thereof, of Formula (V) wherein R⁴ is H. One embodiment provides for a compound, or a pharmaceutically acceptable salt thereof, of Formula (V) wherein R⁵ is H.

One embodiment provides for a compound, or a pharmaceutically acceptable salt thereof, of Formula (V) wherein X¹, X² and X³ are CR⁶ and X⁴ is N. One embodiment provides for a compound, or a pharmaceutically acceptable salt thereof, of Formula (V) wherein X¹, X² and X⁴ are CR⁶ and X³ is N. One embodiment provides for a compound, or a pharmaceutically acceptable salt thereof, of Formula (V) wherein X¹, X³ and X⁴ are CR⁶ and X² is N. One embodiment provides for a compound, or a pharmaceutically acceptable salt thereof, of Formula (V) wherein X², X³ and X⁴ are CR⁶ and X¹ is N. One embodiment provides for a compound, or a pharmaceutically acceptable salt thereof, of Formula (V) wherein X¹ and X² is CR⁶, X³ and X⁴ is N. One embodiment provides for a compound, or a pharmaceutically acceptable salt thereof, of Formula (V) wherein X¹ and X³ is CR⁶, X² and X⁴ is N. One embodiment provides for a compound, or a pharmaceutically acceptable salt thereof, of Formula (V) wherein X¹ and X⁴ is CR⁶, X³ and X² is N. One embodiment provides for a compound, or a pharmaceutically acceptable salt thereof, of Formula (V) wherein X² and X³ is CR⁶, X¹ and X⁴ is N. One embodiment provides for a compound, or a pharmaceutically acceptable salt thereof, of Formula (V) wherein X² and X⁴ is CR⁶, X¹ and X³ is N. One embodiment provides for a compound, or a pharmaceutically acceptable salt thereof, of Formula (V) wherein X³ and X⁴ is CR⁶, X¹ and X² is N.

One embodiment provides for a compound, or a pharmaceutically acceptable salt thereof, of Formula (V) wherein X⁵ is C—Z. One embodiment provides for a compound, or a pharmaceutically acceptable salt thereof, of Formula (V) wherein X⁵ is N. One embodiment provides for a compound, or a pharmaceutically acceptable salt thereof, of Formula (V) wherein R⁶ is halogen. One embodiment provides for a compound, or a pharmaceutically acceptable salt thereof, of Formula (V) wherein R⁶ is H. One embodiment provides for a compound, or a pharmaceutically acceptable salt thereof, of Formula (V) wherein R⁶ is —OH.

One embodiment provides for a compound, or a pharmaceutically acceptable salt thereof, of Formula (V) wherein Y¹, Y² and Y³ are CR⁷ and Y⁴ is N. One embodiment provides for a compound, or a pharmaceutically acceptable salt thereof, of Formula (V) wherein Y¹, Y² and Y⁴ are CR⁷ and Y³ is N. One embodiment provides for a compound, or a pharmaceutically acceptable salt thereof, of Formula (V) wherein Y¹, Y³ and Y⁴ are CR⁷ and Y² is N. One embodiment provides for a compound, or a pharmaceutically acceptable salt thereof, of Formula (V) wherein Y², Y³ and Y⁴ are CR⁷ and Y¹ is N. One embodiment provides for a compound, or a pharmaceutically acceptable salt thereof, of Formula (V) wherein Y¹ and Y² is CR⁷, Y³ and Y⁴ is N. One embodiment provides for a compound, or a pharmaceutically acceptable salt thereof, of Formula (V) wherein Y¹ and Y³ is CR⁷, Y² and Y⁴ is N. One embodiment provides for a compound, or a pharmaceutically acceptable salt thereof, of Formula (V) wherein Y¹ and Y⁴ is CR⁷, Y³ and Y² is N. One embodiment provides for a compound, or a pharmaceutically acceptable salt thereof, of Formula (V) wherein Y² and Y³ is CR⁷, Y¹ and Y⁴ is N. One embodiment provides for a compound, or a pharmaceutically acceptable salt thereof, of Formula (V) wherein Y² and Y⁴ is CR⁷, Y¹ and Y³ is N. One embodiment provides for a compound, or a pharmaceutically acceptable salt thereof, of Formula (V) wherein Y³ and Y⁴ is CR⁷, Y¹ and Y² is N.

One embodiment provides for a compound, or a pharmaceutically acceptable salt thereof, of Formula (V) wherein R⁷ is halogen. One embodiment provides for a compound, or a pharmaceutically acceptable salt thereof, of Formula (V) wherein R⁷ is H. One embodiment provides for a compound, or a pharmaceutically acceptable salt thereof, of Formula (V) wherein R⁷ is —OH.

One embodiment provides for a compound, or a pharmaceutically acceptable salt thereof, of Formula (V) wherein Z is H. One embodiment provides for a compound, or a pharmaceutically acceptable salt thereof, of Formula (V) wherein Z is haloalkyl. One embodiment provides for a compound, or a pharmaceutically acceptable salt thereof, of Formula (V) wherein Z is hydroxyalkyl.

One embodiment provides for a compound, or a pharmaceutically acceptable salt thereof, of Formula (V) wherein Z is -L1-G. One embodiment provides for a compound, or a pharmaceutically acceptable salt thereof, of Formula (V) wherein L1 is a bond. One embodiment provides for a compound, or a pharmaceutically acceptable salt thereof, of Formula (V) wherein L1 is a C₁-C₄ alkylene. One embodiment provides for a compound, or a pharmaceutically acceptable salt thereof, of Formula (V) wherein L1 is C₁ alkylene. One embodiment provides for a compound, or a pharmaceutically acceptable salt thereof, of Formula (V) wherein L1 is —CH₂—. One embodiment provides for a compound, or a pharmaceutically acceptable salt thereof, of Formula (V) wherein G is —N(R¹³)₂—.

One embodiment provides for a compound, or a pharmaceutically acceptable salt thereof, of Formula (V) wherein R¹³ is H. One embodiment provides for a compound, or a pharmaceutically acceptable salt thereof, of Formula (V) wherein R¹³ is optionally substituted alkyl. One embodiment provides for a compound, or a pharmaceutically acceptable salt thereof, of Formula (V) wherein R¹³ is hydroxy substituted alkyl. One embodiment provides for a compound, or a pharmaceutically acceptable salt thereof, of Formula (V) wherein R¹³ is optionally substituted heterocycle. One embodiment provides for a compound, or a pharmaceutically acceptable salt thereof, of Formula (V) wherein R¹³ is alkoxy substituted heterocycle. One embodiment provides for a compound, or a pharmaceutically acceptable salt thereof, of Formula (V) wherein R¹³ is optionally substituted cycloalkyl.

One embodiment provides for a compound, or a pharmaceutically acceptable salt thereof, of Formula (V) wherein W is a bond. One embodiment provides for a compound, or a pharmaceutically acceptable salt thereof, of Formula (V) wherein W is —C≡C—C≡C—. One embodiment provides for a compound, or a pharmaceutically acceptable salt thereof, of Formula (V) wherein W is *—CR¹═CR³—. One embodiment provides for a compound, or a pharmaceutically acceptable salt thereof, of Formula (V) wherein W is *—CR¹R²—CR³R⁴—. One embodiment provides for a compound, or a pharmaceutically acceptable salt thereof, of Formula (V) wherein W is —S—. One embodiment provides for a compound, or a pharmaceutically acceptable salt thereof, of Formula (V) wherein W is —O—. One embodiment provides for a compound, or a pharmaceutically acceptable salt thereof, of Formula (V) wherein W is —CR¹R³—.

One embodiment provides for a compound, or a pharmaceutically acceptable salt thereof, having the structure of Formula (VI):

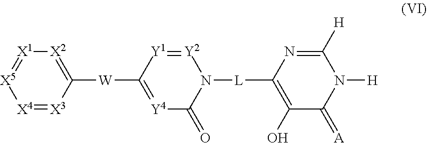

wherein:
A is O or S;
L is *—CR¹R²—CR³R⁴—; *—N(R⁵)—CR³R⁴—; *—O—CR³R⁴—; *—S—CR³R⁴—; *—N(R⁵)—C(O)—; *—C(O)—N(R⁵)—; *—N(R⁵)—S(O)₂—; *—S(O)₂—N(R⁵)—,

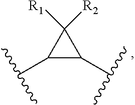

wherein the * denotes a bond to the hydroxypyrimidin-4(3H)-one ring or hydroxypyrimidin-4(3H)-thione ring;
R¹ or R³ are each independently H, optionally alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₁-C₄ alkylene)N(R¹²)—SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
each R⁵ is independently H or optionally substituted alkyl;
each X¹, X², X³, or X⁴ is N, N⁺—O⁻, or C—R⁶;
X⁵ is N, N⁺—O⁻, or C—R⁷;
each R⁶ is independently H, halogen, —OH, or optionally substituted C₁-C₃ alkyl; each Y¹, Y², Y³, or Y⁴ is N or C—R⁷;
R⁷ is halogen, —OH, or optionally substituted C₁-C₃ alkyl;
Z is H, -L1-G, optionally substituted alkyl, optionally substituted carbocyclyl, optionally substituted aryl, optionally substituted heteroaryl, optionally substituted heterocyclyl;
L1 is a bond, optionally substituted C₁-C₄ alkylene, —C(O)—, or —SO₂—;
G is optionally substituted heterocyclyl, optionally substituted heteroaryl, —N(R¹³)₂, —OR¹³, —CN, —OCON(R¹³)₂, or —N(R¹⁴)CON(R¹³)₂;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
W is a bond, —S—, —SO—, —SO₂—, —O—, —CR⁴R³—, —C≡C—C≡C—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR⁴R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the X groups.

One embodiment provides for a compound, or a pharmaceutically acceptable salt thereof, of Formula (VI) wherein A is O.

One embodiment provides for a compound, or a pharmaceutically acceptable salt thereof, of Formula (VI) wherein L is *—CR¹R²—CR³R⁴—. One embodiment provides for a compound, or a pharmaceutically acceptable salt thereof, of Formula (VI) wherein L is *—N(R⁵)—CR³R⁴—. One embodiment provides for a compound, or a pharmaceutically acceptable salt thereof, of Formula (VI) wherein L is *—CR¹R²—N(R⁵)—. One embodiment provides for a compound, or a pharmaceutically acceptable salt thereof, of Formula (VI) wherein L is *—CR¹R²—O—. One embodiment provides for a compound, or a pharmaceutically acceptable salt thereof, of Formula (VI) wherein L is *—CR¹R²—S—. One embodiment provides for a compound, or a pharmaceutically acceptable salt thereof, of Formula (VI) wherein L is *—N(R⁵)—C(O)—. One embodiment provides for a compound, or a pharmaceutically acceptable salt thereof, of Formula (VI) wherein L is *—C(O)—N(R⁵)—. One embodiment provides fora compound, or a pharmaceutically acceptable salt thereof, of Formula (VI) wherein L is *—N(R⁵)—S(O)₂—.

One embodiment provides for a compound, or a pharmaceutically acceptable salt thereof, of Formula (VI) wherein R¹ or R³ is —COR¹¹. One embodiment provides for a compound, or a pharmaceutically acceptable salt thereof, of Formula (VI) wherein R¹ or R³ is —CON(R¹¹)₂. One embodiment provides for a compound, or a pharmaceutically acceptable salt thereof, of Formula (VI) wherein R¹ or R³ is optionally substituted (C₁-C₄ alkylene)-N(R¹¹)₂. One embodiment provides for a compound, or a pharmaceutically acceptable salt thereof, of Formula (VI) wherein R¹ or R³ is optionally substituted (C₁-C₄ alkylene)N(R¹²)—COR¹¹. One embodiment provides for a compound, or a pharmaceutically acceptable salt thereof, of Formula (VI) wherein R¹ or R³ is optionally substituted (C₁-C₄ alkylene)N(R¹²)—CO₂R¹¹.

One embodiment provides for a compound, or a pharmaceutically acceptable salt thereof, of Formula (VI) wherein R² is H. One embodiment provides for a compound, or a pharmaceutically acceptable salt thereof, of Formula (VI) wherein R⁴ is H. One embodiment provides for a compound, or a pharmaceutically acceptable salt thereof, of Formula (VI) wherein R⁵ is H.

One embodiment provides for a compound, or a pharmaceutically acceptable salt thereof, of Formula (VI) wherein X¹, X² and X³ are CR⁶ and X⁴ is N. One embodiment provides for a compound, or a pharmaceutically acceptable salt thereof, of Formula (VI) wherein X¹, X² and X⁴ are CR⁶ and X³ is N. One embodiment provides for a compound, or a pharmaceutically acceptable salt thereof, of Formula (VI) wherein X¹, X³ and X⁴ are CR⁶ and X² is N. One embodiment provides for a compound, or a pharmaceutically acceptable salt thereof, of Formula (VI) wherein X², X³ and X⁴ are CR⁶ and X¹ is N. One embodiment provides for a compound, or a pharmaceutically acceptable salt thereof, of Formula (VI) wherein X¹ and X² is CR⁶, X³ and X⁴ is N. One embodiment provides for a compound, or a pharmaceutically acceptable salt thereof, of Formula (VI) wherein X¹ and X³ is CR⁶, X² and X⁴ is N. One embodiment provides for a compound, or a pharmaceutically acceptable salt thereof, of Formula (VI) wherein X¹ and X⁴ is CR⁶, X³ and X² is N. One embodiment provides for a compound, or a pharmaceutically acceptable salt thereof, of Formula (VI) wherein X² and X³ is CR⁶, X¹ and X⁴ is N. One embodiment provides for a compound, or a pharmaceutically acceptable salt thereof, of Formula (VI) wherein X² and X⁴ is CR⁶, X¹ and X³ is N. One embodiment provides for a compound, or a pharmaceutically acceptable salt thereof, of Formula (VI) wherein X³ and X⁴ is CR⁶, X¹ and X² is N.

One embodiment provides for a compound, or a pharmaceutically acceptable salt thereof, of Formula (VI) wherein X⁵ is C—Z. One embodiment provides for a compound, or a pharmaceutically acceptable salt thereof, of Formula (VI) wherein X⁵ is N.

One embodiment provides for a compound, or a pharmaceutically acceptable salt thereof, of Formula (VI) wherein R⁶ is halogen. One embodiment provides for a compound, or a pharmaceutically acceptable salt thereof, of Formula (VI) wherein R⁶ is H. One embodiment provides for a compound, or a pharmaceutically acceptable salt thereof, of Formula (VI) wherein R⁶ is —OH.

One embodiment provides for a compound, or a pharmaceutically acceptable salt thereof, of Formula (VI) wherein Y¹ and Y² is CR⁷, Y⁴ is N. One embodiment provides for a compound, or a pharmaceutically acceptable salt thereof, of Formula (VI) wherein Y¹ and Y⁴ is CR⁷, Y² is N. One embodiment provides for a compound, or a pharmaceutically acceptable salt thereof, of Formula (VI) wherein Y² and Y⁴ is CR⁷, Y¹ is N. One embodiment provides for a compound, or a pharmaceutically acceptable salt thereof, of Formula (VI) wherein Y¹ is CR⁷, Y² and Y⁴ is N. One embodiment provides for a compound, or a pharmaceutically acceptable salt thereof, of Formula (VI) wherein Y² is CR⁷, Y¹ and Y⁴ is N. One embodiment provides for a compound, or a pharmaceutically acceptable salt thereof, of Formula (VI) wherein Y⁴ is CR⁷, Y¹ and Y² is N.

One embodiment provides for a compound, or a pharmaceutically acceptable salt thereof, of Formula (VI) wherein W is a bond. One embodiment provides for a compound, or a pharmaceutically acceptable salt thereof, of Formula (VI) wherein W is —C≡C—C≡C—. One embodiment provides for a compound, or a pharmaceutically acceptable salt thereof, of Formula (VI) wherein W is *—CR¹═CR³—. One embodiment provides for a compound, or a pharmaceutically acceptable salt thereof, of Formula (VI) wherein W is *—CR¹R²—CR³R⁴—. One embodiment provides for a compound, or a pharmaceutically acceptable salt thereof, of Formula (VI) wherein W is —S—. One embodiment provides for a compound, or a pharmaceutically acceptable salt thereof, of Formula (VI) wherein W is —O—. One embodiment provides for a compound, or a pharmaceutically acceptable salt thereof, of Formula (VI) wherein W is —CR¹R³—.

One embodiment provides for a compound, or a pharmaceutically acceptable salt thereof, of Formula (VI) wherein R⁷ is halogen. One embodiment provides for a compound, or a pharmaceutically acceptable salt thereof, of Formula (VI) wherein R⁷ is H. One embodiment provides for a compound, or a pharmaceutically acceptable salt thereof, of Formula (VI) wherein R⁷ is —OH.

One embodiment provides for a compound, or a pharmaceutically acceptable salt thereof, of Formula (VI) wherein Z is H. One embodiment provides for a compound, or a pharmaceutically acceptable salt thereof, of Formula (VI) wherein Z is haloalkyl. One embodiment provides for a compound, or a pharmaceutically acceptable salt thereof, of Formula (VI) wherein Z is hydroxyalkyl. One embodiment provides for a compound, or a pharmaceutically acceptable salt thereof, of Formula (VI) wherein Z is -L1-G.

One embodiment provides for a compound, or a pharmaceutically acceptable salt thereof, of Formula (VI) wherein L1 is a bond. One embodiment provides for a compound, or a pharmaceutically acceptable salt thereof, of Formula (VI) wherein L1 is a C₁-C₄ alkylene. One embodiment provides for a compound, or a pharmaceutically acceptable salt thereof, of Formula (VI) wherein L1 is C₁ alkylene. One embodiment provides for a compound, or a pharmaceutically acceptable salt thereof, of Formula (VI) wherein L1 is —CH₂—.

One embodiment provides for a compound, or a pharmaceutically acceptable salt thereof, of Formula (VI) wherein G is —N(R¹³)₂—.

One embodiment provides for a compound, or a pharmaceutically acceptable salt thereof, of Formula (VI) wherein R¹³ is H. One embodiment provides for a compound, or a pharmaceutically acceptable salt thereof, of Formula (VI) wherein R¹³ is optionally substituted alkyl. One embodiment provides for a compound, or a pharmaceutically acceptable salt thereof, of Formula (VI) wherein R¹³ is hydroxy substituted alkyl. One embodiment provides for a compound, or a pharmaceutically acceptable salt thereof, of Formula (VI) wherein R¹³ is optionally substituted heterocycle. One embodiment provides for a compound, or a pharmaceutically acceptable salt thereof, of Formula (VI) wherein R¹³ is alkoxy substituted heterocycle. One embodiment provides for a compound, or a pharmaceutically acceptable salt thereof, of Formula (VI) wherein R¹³ is optionally substituted cycloalkyl.

Some embodiments provided herein describe a compound, or a pharmaceutically acceptable salt thereof, having the structure of Formula (VII):

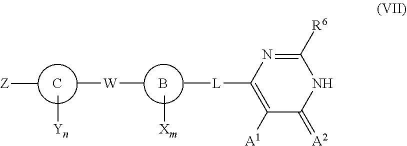

wherein:
n is 0, 1, or 2;
m is 0, 1, or 2;
A¹ is OH or SH;
A² is O or S;
L is a bond, —CR¹R², —N(R⁵)—, —O—, —S—, —SO₂—, *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR¹R²—S—; *—N(R⁵)—C(O)—; *—C(O)—N(R⁵)—; *—N(R⁵)—S(O)₂—; *—S(O)₂—N(R⁵)—; or

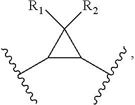

wherein the * denotes a bond to the pyrimidin-4(3H)-one ring or pyrimidin-4(3H)-thione ring;
R¹, R², R³, R⁴, and R⁵ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₀-C₄ alkylene)-CN, optionally substituted (C₀-C₄ alkylene)-OR¹¹, optionally substituted (C₀-C₄ alkylene)-N(R¹¹)₂, optionally substituted (C₀-C₄ alkylene)N(R¹²)—COR¹¹, optionally substituted (C₀-C₄ alkylene)N(R¹²)—CO₂R¹¹, optionally substituted (C₀-C₄ alkylene)N(R¹²)—CON(R¹¹)₂, optionally substituted (C₀-C₄ alkylene)N(R¹²)—SO₂N(R¹¹)₂, optionally substituted (C₀-C₄ alkylene)-O—SO₂N(R¹¹)₂, optionally substituted (C₀-C₄ alkylene)-N(R¹²)—SO₂R¹¹, optionally substituted (C₀-C₄ alkylene)-SO₂R¹¹, or optionally substituted (C₀-C₄ alkylene)N(R¹¹)—PO (optionally substituted C₀-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
   R⁶ is H, halogen, optionally substituted alkyl, hydroxyl, alkoxyl, cyano, amino, or nitro;

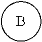

is phenyl,

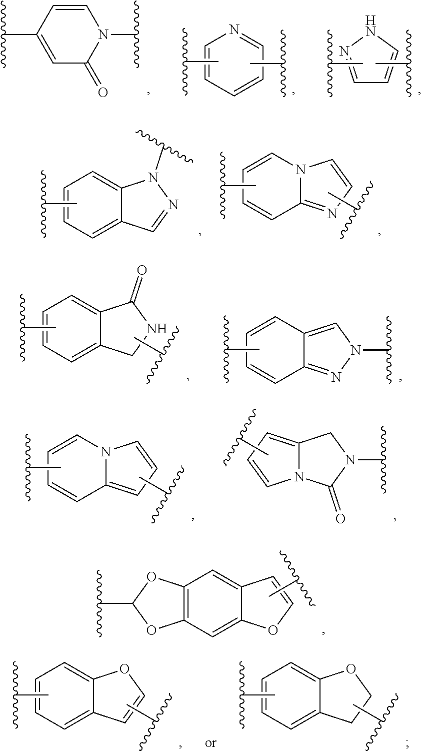

W is a bond, —C≡C—, —C≡C—C≡C—, —S—, —SO—, —SO₂—, —O—, —CR¹R³—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the Y groups;

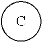

is absent, phenyl, alkyl, cycloalkyl,

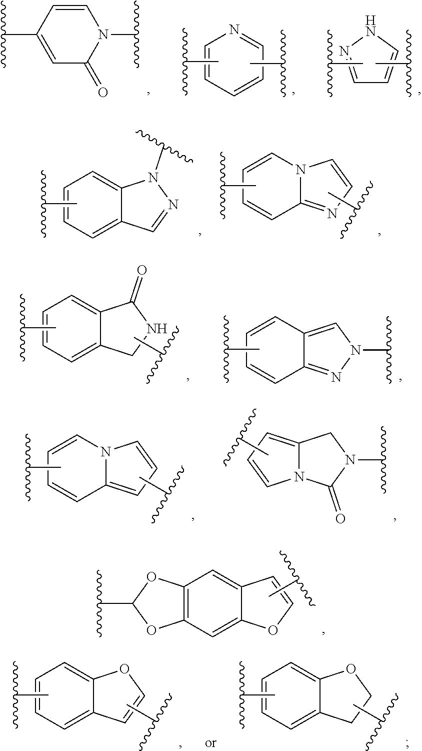

provided that if

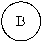

is phenyl or pyridine, then

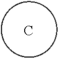

is not phenyl or pyridine; and
if

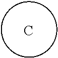

is phenyl or pyridine, then

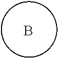

is not phenyl or pyridine;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L2-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L2 is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

Certain embodiments provided herein describe a compound, or a pharmaceutically acceptable salt thereof, having the structure of Formula (VIIa):

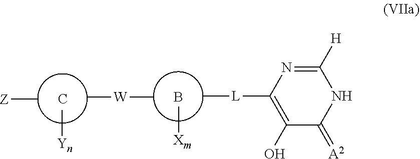

wherein:
n is 0, 1, or 2;
m is 0, 1, or 2;
A² is O or S;
L is —CR¹R²—, —N(R⁵)—, —O—, —S—, —SO₂—, *—CR¹R²—CR³R⁴—; *—N(R⁵)—CR³R⁴—; *—CR¹R²—N(R⁵)—; *—O—CR³R⁴—;*—CR¹R²—O—; *—S—CR³R⁴—; *—CR¹R²—S—; *—N(R⁵)—C(O)—; *—C(O)—N(R⁵)—; *—N(R⁵)—S(O)₂—; *—S(O)₂—N(R⁵)—; or

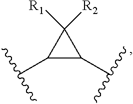

wherein the * denotes a bond to the hydroxypyrimidin-4(3H)-one ring or hydroxypyrimidin-4(3H)-thione ring;
R¹ or R³ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₀-C₄ alkylene)-CN, optionally substituted (C₀-C₄ alkylene)-OR¹¹, optionally substituted (C₀-C₄ alkylene)-N(R¹¹)₂, optionally substituted (C₀-C₄ alkylene)N(R¹²)—COR¹¹, optionally substituted (C₀-C₄ alkylene)N(R¹²)—CO₂R¹¹, optionally substituted (C₀-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₀-C₄ alkylene)N(R¹²)—SO₂N(R¹¹)₂, optionally substituted (C₀-C₄ alkylene)N(R¹²)—SO₂R¹¹, optionally substituted (C₀-C₄ alkylene)-O—SO₂N(R¹¹)₂, or optionally substituted (C₀-C₄ alkylene)N(R¹¹)—PO (optionally substituted C₁-C₄ alkyl)₂;
R², R⁴, and R⁵ are each independently H or optionally substituted C₁-C₃ alkyl;

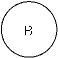

is phenyl,

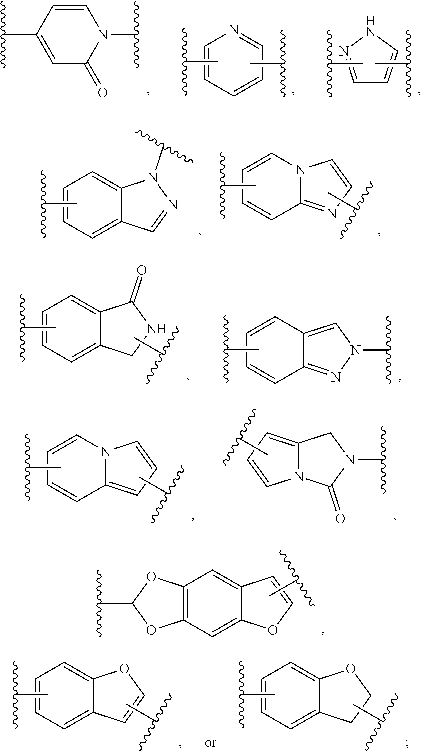

W is a bond, —C≡C—, —C≡C—C≡C—, —S—, —SO—, —SO₂—, —O—, —CR⁴R³—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the Y groups:

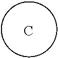

is absent, phenyl, alkyl, cycloalkyl,

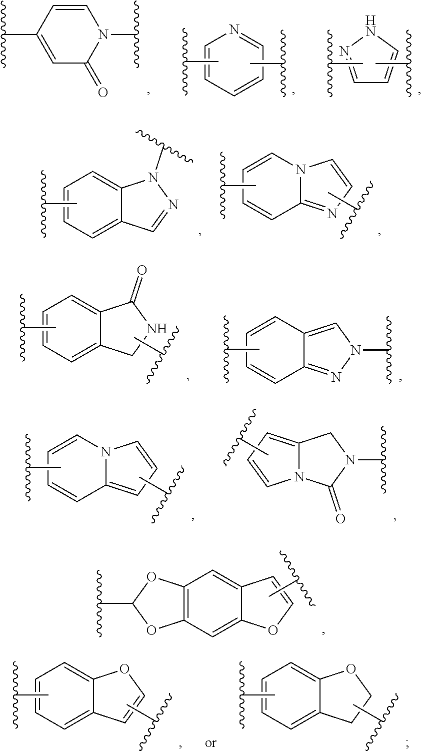

provided that if

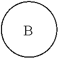

is phenyl or pyridine, then

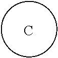

is not phenyl or pyridine; and
if

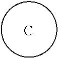

is phenyl or pyridine, then

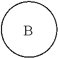

is not phenyl or pyridine;
X is halogen or optionally substituted C₁-C₃ alkyl;
Y is halogen or optionally substituted C₁-C₃ alkyl;
Z is H, optionally substituted alkyl, -L2-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂;
L2 is a bond, optionally substituted C₁-C₄ alkylene, —C(O)—, or —SO₂—;
G is optionally substituted heterocyclyl, optionally substituted heteroaryl, —N(R¹³)₂, —OR¹³, halogen, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

In some embodiments,
n is 0 or 1;
m is 0 or 1;
A² is O or S;
L is —CR¹R²—, *—CR¹R²—CR³R⁴—; *—CR¹R²—N(R⁵)—; *—CR⁴R²—O—; *—CR¹R²—S—; *—N(R⁵)—C(O)—; or *—C(O)—N(R⁵)—, wherein the * denotes a bond to the hydroxypyrimidin-4(3H)-one ring or hydroxypyrimidin-4(3H)-thione ring;
R¹ or R³ are each independently H, optionally substituted alkyl, —COR¹¹, —CON(R¹¹)₂, (C₀-C₄ alkylene)-CN, (C₀-C₄ alkylene)-OR¹¹, (C₀-C₄ alkylene)-N(R¹¹)₂, (C₀-C₄ alkylene)-SO₂N(R¹¹)₂, (C₀-C₄ alkylene)-SO₂R¹¹;
R², R⁴, and R⁵ are each independently H or optionally substituted C₁-C₃ alkyl;

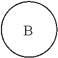

is phenyl,

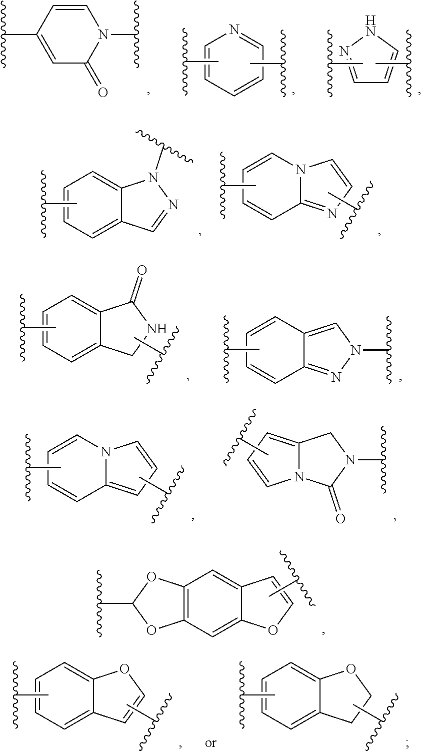

W is a bond, —C≡C—, or —C≡C—C≡C—;

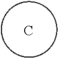

is absent, phenyl, alkyl, cycloalkyl,

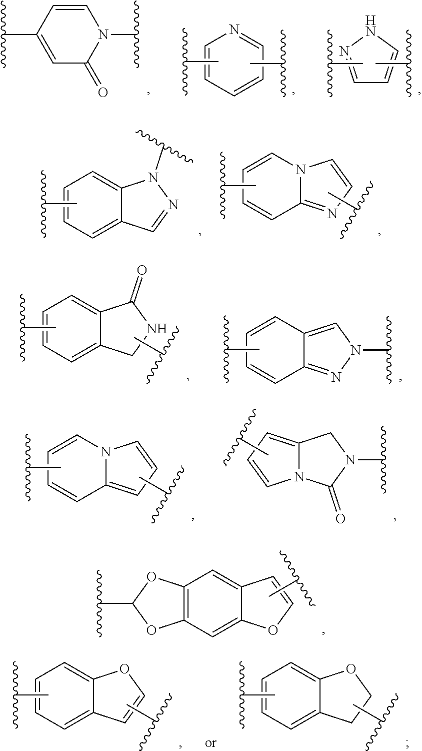

provided that if

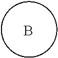

is phenyl or pyridine, then

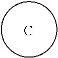

is not phenyl or pyridine; and
if

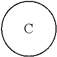

is phenyl or pyridine, then

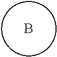

is not phenyl or pyridine;
X is halogen or optionally substituted C₁-C₃ alkyl;
Y is halogen or optionally substituted C₁-C₃ alkyl;
Z is H, alkyl, -L2-G, (C₁-C₄ alkylene)-OCON(R¹³)₂, (C₁-C₄ alkylene)-N(R¹⁴)CON(R¹³)₂, or (C₁-C₄ alkylene)-SO₂N(R¹³)₂;
L2 is a bond, C₁-C₄ alkylene, —C(O)—, or —SO₂—;
G is optionally substituted heterocyclyl, —N(R¹³)₂, —OR¹³, halogen, or —CN;
each R¹¹ is independently H, optionally substituted alkyl, optionally substituted alkenyl; or two R¹¹ groups together with the nitrogen to which they are attached join to form an optionally substituted N-heterocyclyl;
each R¹² is independently H or alkyl;
each R¹³ is independently H, optionally substituted alkyl, optionally substituted carbocyclyl, optionally substituted carbocyclylalkyl, optionally substituted heterocyclyl, optionally substituted heterocyclylalkyl; or two R¹³ groups together with the nitrogen to which they are attached join to form an optionally substituted N-heterocyclyl; and
each R¹⁴ is independently H or alkyl.

In some embodiments,
n is 0 or 1;
m is 0 or 1;
A² is O;
L is —CR¹R²—, *—CR¹R²—CR³R⁴—; *—CR¹R²—N(R⁵)—; or *—CR¹R²—S—; wherein the * denotes a bond to the hydroxypyrimidin-4(3H)-one ring or hydroxypyrimidin-4(3H)-thione ring;
R¹ or R³ are each independently H, optionally substituted alkyl, —COR¹¹, —CON(R¹¹)₂, or (C₀-C₄ alkylene) —N(R¹¹)₂;
R², R⁴, and R⁵ are each independently H;

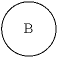

is phenyl,

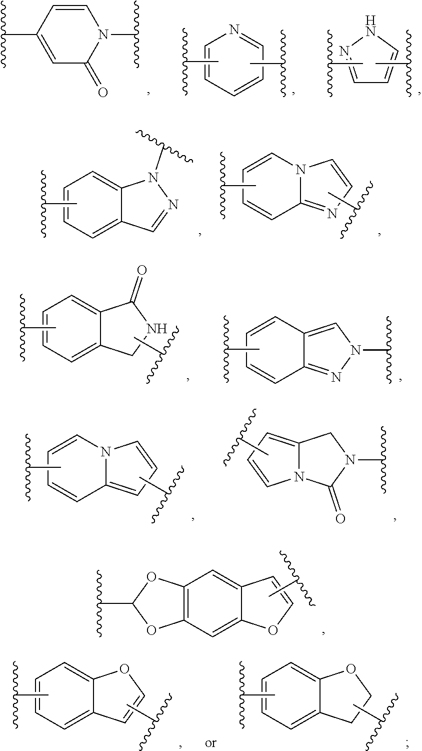

W is a bond, —C≡C—, or —C≡C—C≡C—;

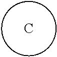

is absent, phenyl, alkyl, cycloalkyl,

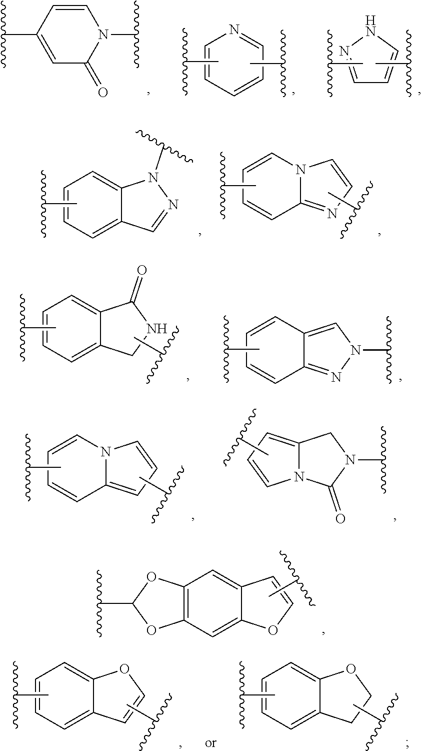

provided that if

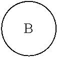

is phenyl or pyridine, then

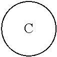

is not phenyl or pyridine; and
if

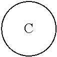

is phenyl or pyridine, then

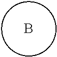

is not phenyl or pyridine;
X is F;
Y is F;
Z is H, C₁-C₆ alkyl, -L2-G;
L2 is a bond, C₁-C₄ alkylene, or —C(O)—;
G is heterocyclyl, —N(R¹³)₂, —OR¹³, F, Br, Cl, or —CN;
each R¹¹ is independently H, C₁-C₆ alkyl, C₁-C₆ fluoroalkyl, or C₁-C₆ alkenyl; or two R¹¹ groups together with the nitrogen to which they are attached join to form an N-heterocyclyl;
each R¹² is H;
each R¹³ is H, C₁-C₆ alkyl, C₃-C₆ carbocyclyl, C₃-C₈ carbocyclylalkyl, C₂-C₆ heterocyclyl, or C₂-C₈ heterocyclylalkyl; or two R¹³ groups together with the nitrogen to which they are attached join to form an N-heterocyclyl; and
each R¹⁴ is H.

For any and all of the embodiments of Formula (VII) or (VIIa), substituents are selected from among a subset of the listed alternatives.

In some embodiments, L is *—CR¹R²—CR³R⁴—; *—CR¹R²—N(R⁵)—; or *—C(O)—N(R⁵)—. In some embodiments, L is *—CR¹R²—CR³R⁴— or *—CR¹R²—N(R⁵)—. In some embodiments, L is *—CR¹R²—CR³R⁴— or *—C(O)—N(R⁵)—. In some embodiments, L is *—CR¹R²—CR³R⁴—. In some embodiments, L is *—CR¹R²—N(R⁵)—. In some embodiments, L is *—C(O)—N(R⁵)—.

In some embodiments, R¹ or R³ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)-N(R¹²)—SO₂R¹¹, optionally substituted (C₁-C₄ alkylene)-SO₂R¹¹, or optionally substituted (C₁-C₄ alkylene)N(R¹¹)—PO (optionally substituted C₁-C₄ alkyl)₂. In some embodiments, R¹ or R³ are each independently H, optionally substituted (C₁-C₄ alkylene)-OR¹¹, optionally substituted (C₁-C₄ alkylene)-N(R¹¹)₂, optionally substituted (C₁-C₄ alkylene)-N(R¹²)—SO₂R¹¹, or optionally substituted (C₁-C₄ alkylene)-SO₂R¹¹. In some embodiments, R¹ or R³ are each independently H, (C₁-C₄ alkylene)-OH, (C₁-C₄ alkylene)-NH₂, (C₁-C₄ alkylene)-NH—SO₂Me, or (C₁-C₄ alkylene)-SO₂Me.

In some embodiments, R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)-N(R¹²)—SO₂R¹¹, optionally substituted (C₁-C₄ alkylene)-SO₂R¹¹, or optionally substituted (C₁-C₄ alkylene)N(R¹¹)—PO (optionally substituted C₁-C₄ alkyl)₂. In some embodiments, R¹ is optionally substituted heterocyclyl, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)-SO₂N(R¹¹)₂, or optionally substituted (C₁-C₄ alkylene)-SO₂R¹¹. In other embodiments, R¹ is optionally substituted (C₁-C₄ alkylene)-OR¹¹, optionally substituted (C₁-C₄ alkylene)-N(R¹¹)₂, optionally substituted (C₁-C₄ alkylene)-N(R¹²)—SO₂R¹¹, or optionally substituted (C₁-C₄ alkylene)-SO₂R¹¹. In some embodiments, R¹ is H, optionally substituted (C₁-C₄ alkylene)-OH, optionally substituted (C₁-C₄ alkylene)-NH₂, optionally substituted (C₁-C₄ alkylene)-NH—SO₂Me, or optionally substituted (C₁-C₄ alkylene)-SO₂Me. In certain embodiments, R¹ is —CON(R¹¹)₂, (C₁-C₄ alkylene)-N(R¹¹)₂, or (C₁-C₄ alkylene)N(R¹²)—COR¹¹. In other embodiments R¹ is —CON(R¹¹)₂, (C₁ alkylene)-N(R¹¹)₂, or (C₁ alkylene)N(R¹²)—COR¹¹. In other embodiments R¹ is —CON(R¹¹)₂, (C₁ alkylene)-N(R¹¹)₂, or (C₁ alkylene)N(R¹²)—COR¹¹, wherein R¹¹ is H, alkyl, or fluoroalkyl; and R¹² is H.

In some embodiments, R³ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)-N(R¹²)—SO₂R¹¹, optionally substituted (C₁-C₄ alkylene)-SO₂R¹¹, or optionally substituted (C₁-C₄ alkylene)N(R¹¹)—PO (optionally substituted C₁-C₄ alkyl)₂. In other embodiments, R³ is optionally substituted (C₁-C₄ alkylene)-OR¹¹, optionally substituted (C₁-C₄ alkylene)-N(R¹¹)₂, optionally substituted (C₁-C₄ alkylene)-N(R¹²)—SO₂R¹¹, or optionally substituted (C₁-C₄ alkylene)-SO₂R¹¹. In some embodiments, R³ is H, optionally substituted (C₁-C₄ alkylene)-OH, optionally substituted (C₁-C₄ alkylene)-NH₂, optionally substituted (C₁-C₄ alkylene)-NH—SO₂Me, or optionally substituted (C₁-C₄ alkylene)-SO₂Me. In certain embodiments, R³ is —CON(R¹¹)₂, (C₁-C₄ alkylene)-N(R¹¹)₂, or (C₁-C₄ alkylene)N(R¹²)—COR¹¹. In other embodiments R³ is —CON(R¹¹)₂, (C₁ alkylene)-N(R¹¹)₂, or (C₁ alkylene)N(R¹²)—COR¹¹. In other embodiments R³ is —CON(R¹¹)₂, (C₁ alkylene)-N(R¹¹)₂, or (C₁ alkylene)N(R¹²)—COR¹¹, wherein R¹¹ is H, alkyl, or fluoroalkyl; and R¹² is H.

In some embodiments, R², R⁴, and R⁵ are each independently H or optionally substituted C₁-C₃ alkyl. In some embodiments, R², R⁴, and R⁵ are each independently H or C₁-C₃ alkyl. In some embodiments, R² is H. In some embodiments, R⁴ is H. In some embodiments, R⁵ is H. In some embodiments, R², R⁴, and R⁵ are H. In some embodiments, R¹ and R² are H. In some embodiments, R³ and R⁴ are H.

In some embodiments, n is 0. In some embodiments, m is 0. In some embodiments, m is 1 or 2. In some embodiments, m is 1. In some embodiments, m is 2. In some embodiments, n is 1 or 2. In some embodiments, n is 1. In some embodiments, n is 2. In some embodiments, X is halogen. In some embodiments, X is F, Cl, or Br. In some embodiments, X is F. In some embodiments, Y is halogen. In some embodiments, Y is F, Cl, or Br. In some embodiments, Y is F.

In some embodiments, Z is H, alkyl, -L2-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂. In some embodiments, Z is -L2-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂. In some embodiments, Z is H or -L2-G, wherein L2 is C₁-C₄ alkylene and G is optionally substituted heterocyclyl. In some embodiments, Z is H. In some embodiments, Z is L2-G, wherein L2 is C₁-C₄ alkylene and G is optionally substituted heterocyclyl. In some embodiments, Z is H, alkyl, -L2-G; wherein L2 is a bond, optionally substituted C₁-C₄ alkylene, —C(O)—; and G is optionally substituted heterocyclyl, —N(R¹³)₂, —OR¹³, halogen, or —CN. In some embodiments, Z is H, alkyl, or -L2-G, wherein L2 is C₁-C₄ alkylene and G is heterocyclyl. In some embodiments, Z is H, alkyl, or -L2-G, wherein L2 is C₁-C₄ alkylene and G is morpholino.

In some embodiments, W is a bond, —C≡C—, —C≡C—C≡C—, or —CH═CH—. In some embodiments, W is a bond, —C≡C—, or —C≡C—C≡C—. In other embodiments, W is a —C≡C— or —C≡C—C≡C—. In some embodiments, W is a bond. In some embodiments, W is —C≡C—. In other embodiments, W is —C≡C—C≡C—.

In some embodiments, R⁶ is H, F, Cl, Br, alkyl, hydroxyl, alkoxyl, cyano, amino, or nitro. In some embodiments, R⁶ is H.

In some embodiments, A¹ is OH and A² is O. In some embodiments, A¹ is SH and A² is O. In some embodiments, A¹ is SH and A² is S. In some embodiments, A¹ is OH and A² is S.

In some embodiments, each R¹¹ is independently H, haloalkyl, alkyl, alkenyl, carbocyclyl, or carbocyclylalkyl; or two R¹¹ groups together with the nitrogen to which they are attached join to form an optionally substituted N-heterocyclyl. In some embodiments, each R¹¹ is independently H, haloalkyl, alkyl, or alkenyl. In some embodiments, each R¹¹ is independently H, fluoroalkyl, alkyl, or alkenyl. In some embodiments, each R¹¹ is independently H, C₁₋₆ fluoroalkyl, C₁₋₆ alkyl, or C₃alkenyl. In some embodiments, each R¹¹ is independently H, C₁₋₃ fluoroalkyl, C₁₋₆ methyl, or C₃alkenyl. In some embodiments, R¹² is H or methyl. In some embodiments, R¹² is H.

In some embodiments, each R¹³ is independently H, optionally substituted alkyl, optionally substituted carbocyclyl, optionally substituted carbocyclylalkyl, optionally substituted heterocyclyl, or optionally substituted heterocyclylalkyl; or two R¹³ groups together with the nitrogen to which they are attached join to form an optionally substituted N-heterocyclyl; and each R¹⁴ is independently H or optionally substituted alkyl. In some embodiments, each R¹³ is independently H, alkyl, carbocyclyl, carbocyclylalkyl, heterocyclyl, or heterocyclylalkyl; or two R¹³ groups together with the nitrogen to which they are attached join to form an N-heterocyclyl; and each R¹⁴ is H. In some embodiments, each R¹³ is independently H or alkyl; or two R¹³ groups together with the nitrogen to which they are attached join to form an N-heterocyclyl; and each R¹⁴ is H. In some embodiments, each R¹³ is independently H, haloalkyl, alkyl, alkenyl, or carbocyclyl, carbocyclylalkyl; or two R¹³ groups together with the nitrogen to which they are attached join to form an optionally substituted N-heterocyclyl. In some embodiments, each R¹³ is independently H, haloalkyl, alkyl, or alkenyl. In some embodiments, each R¹³ is independently H, fluoroalkyl, alkyl, or alkenyl. In some embodiments, each R¹³ is independently H, C₁₋₆ fluoroalkyl, C₁₋₆ alkyl, or C₃alkenyl. In some embodiments, each R¹³ is independently H, C₁₋₃ fluoroalkyl, C₁₋₆ methyl, or C₃alkenyl. In some embodiments, R¹⁴ is H or methyl. In some embodiments, R¹⁴ is H.

In some embodiments,

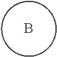

is phenyl,

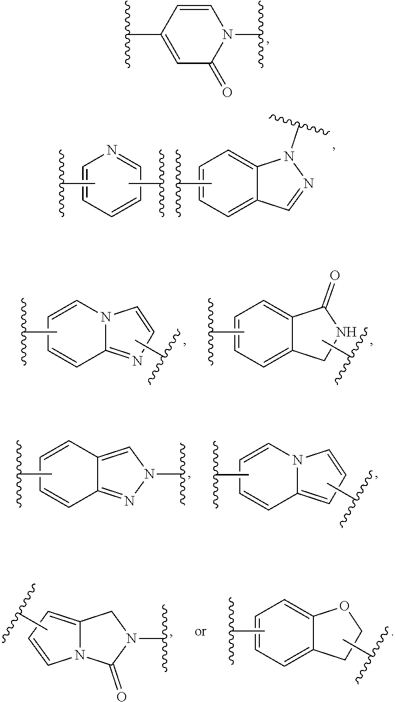

In some embodiments,

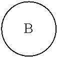

is phenyl. In some embodiments,

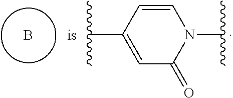

In some embodiments,

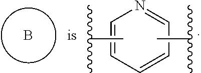

In some embodiments,

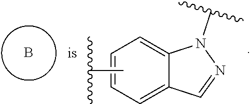

In some embodiments,

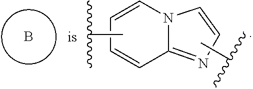

In some embodiments,

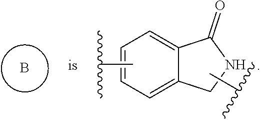

In some embodiments,

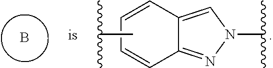

In some embodiments,

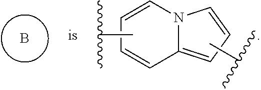

In some embodiments

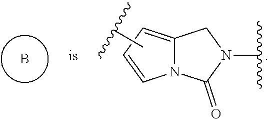

In some embodiments,

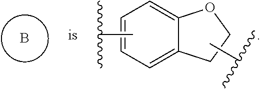

In some embodiments,

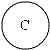

is absent, phenyl, alkyl, cycloalkyl,

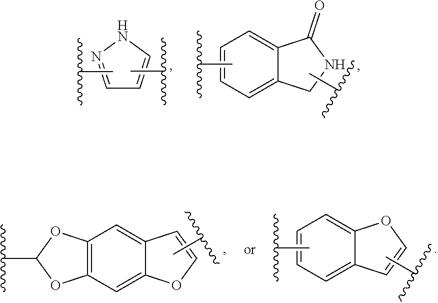

In some embodiments,

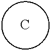

is phenyl, alkyl, cycloalkyl,

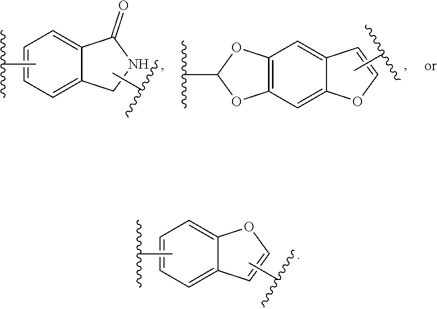

In some embodiments,

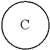

is phenyl, cycloalkyl,

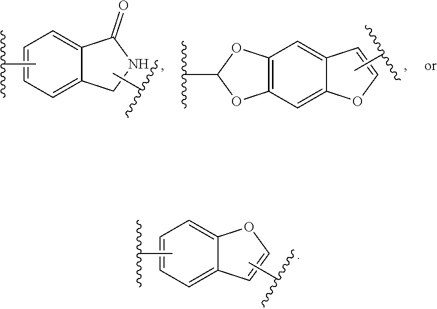

In some embodiments,

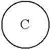

is phenyl. In some embodiments,

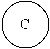

is cycloalkyl. In some embodiments,

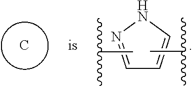

In some embodiments,

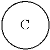

is absent. In some embodiments,

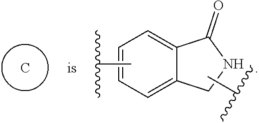

In some embodiments,

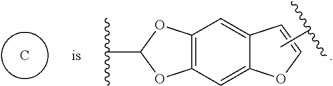

In some embodiments,

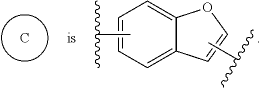

In some embodiments,

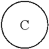

is phenyl or cycloalkyl. In some embodiments,

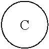

is phenyl or C₃₋₆ cycloalkyl. In some embodiments,

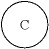

is phenyl or cyclopropyl. In some embodiment, W is —C≡C— or —C≡C—C≡C—; and

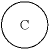

is phenyl or cyclopropyl. In some embodiments,

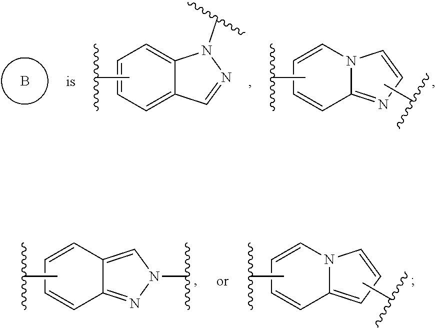

W is bond, —C≡C— or —C≡C—C≡C—; and

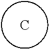

is phenyl or C₃₋₆ cycloalkyl. In some embodiments,

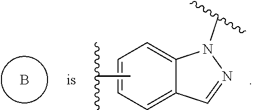

### W is —C≡C—C≡C—; and

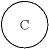

is cyclopropyl.

In some embodiments, a compound of Formula (VII) or (VIIa), or a pharmaceutically acceptable salt thereof, has the structure of Formula (VIIb):

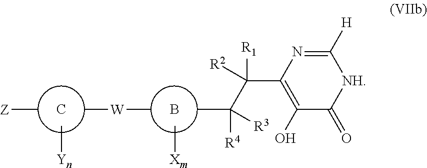

In some embodiments, a compound of Formula (VII) or (VIIa), or a pharmaceutically acceptable salt thereof, has the structure of Formula (VIIe):

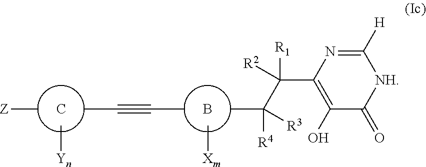

In some embodiments, a compound of Formula (VII) or (VIIa), or a pharmaceutically acceptable salt thereof, has the structure of Formula (VIId):

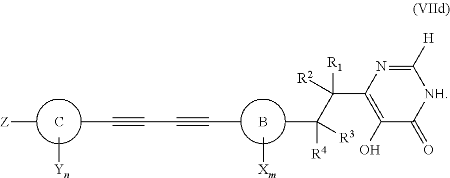

In some embodiments, a compound of Formula (VII) or (VIIa), or a pharmaceutically acceptable salt thereof, has the structure of Formula (VIIe):

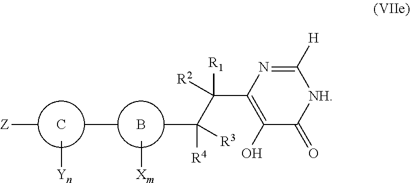

In some embodiments, the heterocyclic LpxC inhibitory compound described herein has a structure provided in Table 1.

TABLE 1

| Compound | | | Mass |
|---|---|---|---|
| No. | Name | Structure | [M + H] |
| 1 | N-(2-(3-hydroxy-2-oxo-1,2- dihydropyridin-4-yl)-3-(4- ((4- (morpholinomethyl)phenyl) ethynyl)phenyl)propyl) acetamide | 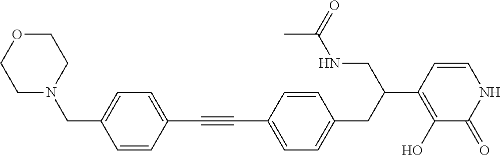 [SMILES: CC(=O)NCC(Cc1ccc(C#Cc2ccc(CN3CCOCC3)cc2)cc1)c1cc[nH]c(=O)c1O] | 486.13 |
| 2 | 3-hydroxy-1-methyl-2-(((4- ((4- (morpholinomethyl)phenyl) ethynyl)phenyl)amino)methyl) pyridin-4(1H)-one | 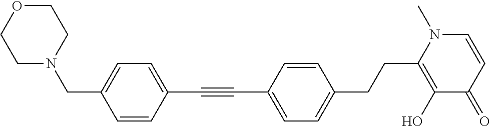 [SMILES: Cn1ccc(=O)c(O)c1CCc1ccc(C#Cc2ccc(CN3CCOCC3)cc2)cc1] | 430.12 |
| 3 | N-(2-(5-hydroxy-6- oxopyrimidin-1(6H)-yl)-3-(4- ((4- (morpholinomethyl)phenyl) ethynyl)phenyl)propyl) acetamide | 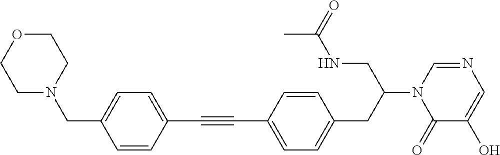 [SMILES: CC(=O)NCC(Cc1ccc(C#Cc2ccc(CN3CCOCC3)cc2)cc1)n1cncc(O)c1=O] | 487.23 |
| 4 | 2-(5-hydroxy-6- oxopyrimidin-1(6H)-yl)-N- methyl-3-(4-((4- (morpholinomethyl)phenyl) ethynyl)phenyl)propanamide | 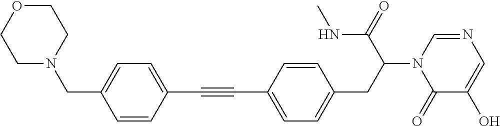 [SMILES: CNC(=O)C(Cc1ccc(C#Cc2ccc(CN3CCOCC3)cc2)cc1)n1cncc(O)c1=O] | 473.24 |
| 5 | 2-(5-hydroxy-6- oxopyrimidin-1(6H)-yl)-3-(4- ((4- (morpholinomethyl)phenyl) ethynyl)phenyl)propanamide | 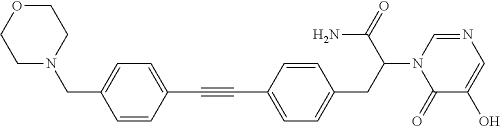 [SMILES: NC(=O)C(Cc1ccc(C#Cc2ccc(CN3CCOCC3)cc2)cc1)n1cncc(O)c1=O] | 459.32 |
| 6 | 2-(3-hydroxy-2-oxopyridin- 1(2H)-yl)-3-(4-((4- (morpholinomethyl)phenyl) ethynyl)phenyl)propanamide | 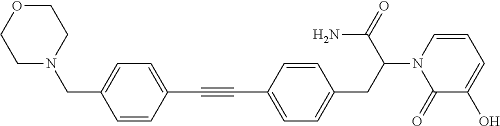 [SMILES: NC(=O)C(Cc1ccc(C#Cc2ccc(CN3CCOCC3)cc2)cc1)n1cccc(O)c1=O] | 458.31 |
| 7 | N-(2-(5-hydroxy-6- oxopyrimidin-1(6H)-yl)-3-(4- ((4- (morpholinomethyl)phenyl) ethynyl)phenyl)propyl) methanesulfonamide | 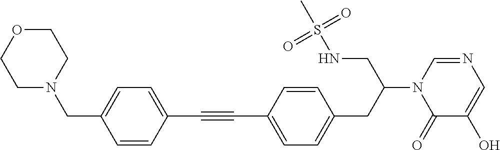 [SMILES: CS(=O)(=O)NCC(Cc1ccc(C#Cc2ccc(CN3CCOCC3)cc2)cc1)n1cncc(O)c1=O] | 523.18 |
| 8 | N-(2-(5-hydroxy-3-oxo-2,3- dihydropyridazin-4-yl)-3-(4- ((4- (morpholinomethyl)phenyl) ethynyl)phenyl)propyl) acetamide | 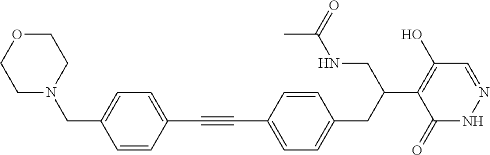 [SMILES: CC(=O)NCC(Cc1ccc(C#Cc2ccc(CN3CCOCC3)cc2)cc1)c1c(O)cn[nH]c1=O] | 487.23 |
| 9 | 2-fluoro-N-(2-(5-hydroxy-1- methyl-6-oxo-1,6- dihydropyrimidin-4-yl)-3-(4- ((4- (morpholinomethyl)phenyl) ethynyl)phenyl)propyl) acetamide | 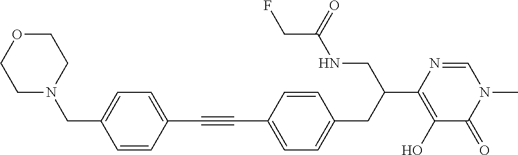 [SMILES: Cn1cnc(C(CNC(=O)CF)Cc2ccc(C#Cc3ccc(CN4CCOCC4)cc3)cc2)c(O)c1=O] | 519.13 |
| 10 | 2-(3-hydroxy-2-oxo-1,2- dihydropyridin-4-yl)-3-(4- ((4- (morpholinomethyl)phenyl) ethynyl)phenyl)propanenitrile | 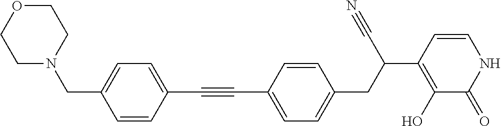 [SMILES: N#CC(Cc1ccc(C#Cc2ccc(CN3CCOCC3)cc2)cc1)c1cc[nH]c(=O)c1O] | 440.18 |
| 11 | N-(2-(4,5- dihydroxypyridazin-3-yl)-3- (4-((4- (morpholinomethyl)phenyl) ethynyl)phenyl)propyl) acetamide | 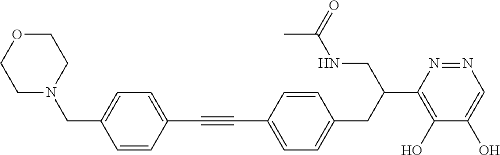 [SMILES: CC(=O)NCC(Cc1ccc(C#Cc2ccc(CN3CCOCC3)cc2)cc1)c1nncc(O)c1O] | 487.22 |
| 12 | 2-(3,4-dihydroxypyridin-2- yl)-N-methyl-3-(4-((4- (morpholinomethyl)phenyl) ethynyl)phenyl)propanamide | 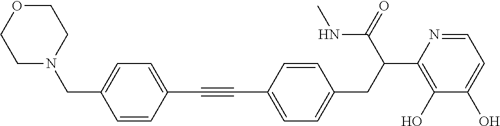 [SMILES: CNC(=O)C(Cc1ccc(C#Cc2ccc(CN3CCOCC3)cc2)cc1)c1nccc(O)c1O] | 472.12 |
| 13 | 6-(2-(5-((4- (((tetrahydrofuran-3- yl)amino)methyl)phenyl) ethynyl)pyridin-2- yl)ethyl)pyrimidine-4,5-diol | 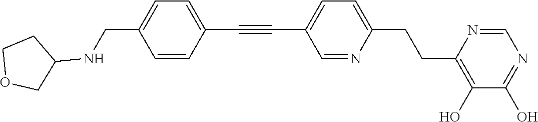 [SMILES: Oc1ncnc(CCc2ccc(C#Cc3ccc(CNC4CCOC4)cc3)cn2)c1O] | |
| 14 | 5-hydroxy-6-(4-(pyrimidin-2- ylethynyl)phenethyl) pyrimidin-4(3H)-one | 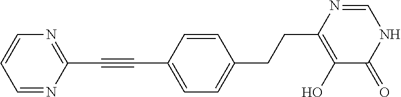 [SMILES: O=c1[nH]cnc(CCc2ccc(C#Cc3ncccn3)cc2)c1O] | 319.13 |
| 15 | 5-hydroxy-6-(2-(5-((4-(((3- hydroxycyclobutyl)amino) methyl)phenyl)ethynyl)pyridin- 2-yl)ethyl)pyrimidin-4(3H)- one | 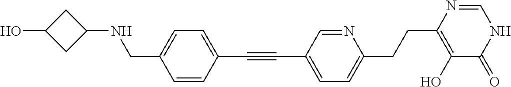 [SMILES: O=c1[nH]cnc(CCc2ccc(C#Cc3ccc(CNC4CC(O)C4)cc3)cn2)c1O] | 417.20 |
| 16 | 5-hydroxy-6-(4-((5- (morpholinomethyl)pyridin- 2-yl)ethynyl)phenethyl) pyrimidin-4(3H)-one | 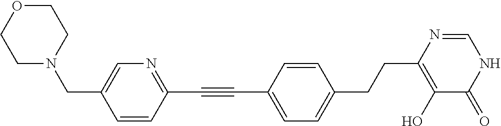 [SMILES: O=c1[nH]cnc(CCc2ccc(C#Cc3ccc(CN4CCOCC4)cn3)cc2)c1O] | 417.14 |
| 17 | 5-hydroxy-6-(4-((6- (morpholinomethyl)pyridin- 3-yl)ethynyl)phenethyl) pyrimidin-4(3H)-one | 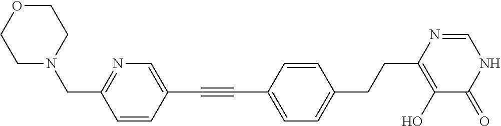 [SMILES: O=c1[nH]cnc(CCc2ccc(C#Cc3ccc(CN4CCOCC4)nc3)cc2)c1O] | 417.15 |
| 18 | 5-hydroxy-6-(4-(pyridin-4- ylethynyl)phenethyl) pyrimidin-4(3H)-one | 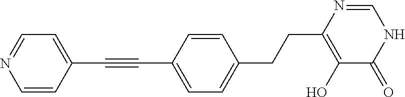 [SMILES: O=c1[nH]cnc(CCc2ccc(C#Cc3ccncc3)cc2)c1O] | 318.32 |
| 19 | 5-hydroxy-6-(2-(5-((4- (morpholinomethyl)phenyl) ethynyl)pyridin-2- yl)ethyl)pyrimidin-4(3H)-one | 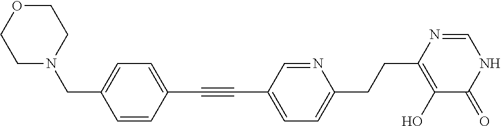 [SMILES: O=c1[nH]cnc(CCc2ccc(C#Cc3ccc(CN4CCOCC4)cc3)cn2)c1O] | 417.21 |
| 20 | 5-hydroxy-6-(2-(5-((4- (((tetrahydrofuran-3- yl)amino)methyl)phenyl) ethynyl)pyridin-2- yl)ethyl)pyrimidin-4(3H)-one | 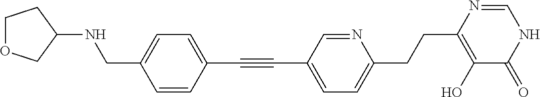 [SMILES: O=c1[nH]cnc(CCc2ccc(C#Cc3ccc(CNC4CCOC4)cc3)cn2)c1O] | 417.32 |
| 21 | 5-hydroxy-6-(2-(5-((4- (morpholinomethyl)phenyl) ethynyl)pyrimidin-2- yl)ethyl)pyrimidin-4(3H)-one | 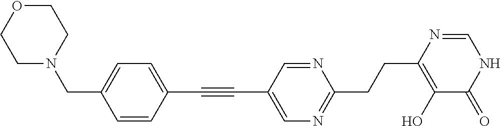 [SMILES: O=c1[nH]cnc(CCc2ncc(C#Cc3ccc(CN4CCOCC4)cc3)cn2)c1O] | 418.19 |
| 22 | 5-hydroxy-6-(4-((5- (((tetrahydrofuran-3- yl)amino)methyl)pyridin-2- yl)ethynyl)phenethyl) pyrimidin-4(3H)-one | 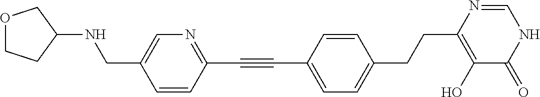 [SMILES: O=c1[nH]cnc(CCc2ccc(C#Cc3ccc(CNC4CCOC4)cn3)cc2)c1O] | 417.31 |
| 23 | 5-hydroxy-6-(2-(4-((4- (morpholinomethyl)phenyl) ethynyl)-2-oxopyridin-1(2H)- yl)ethyl)pyrimidin-4(3H)-one | 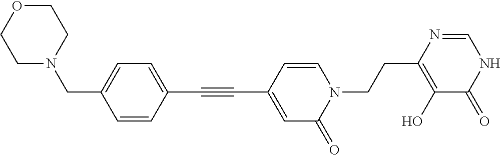 [SMILES: O=c1[nH]cnc(CCn2ccc(C#Cc3ccc(CN4CCOCC4)cc3)cc2=O)c1O] | 433.21 |
| 24 | 3-(4-((2-aminopyrimidin-5- yl)ethynyl)phenyl)-N-(2,2- difluoroethyl)-2-(5-hydroxy- 6-oxo-1,6-dihydropyrimidin- 4-yl)propanamide | 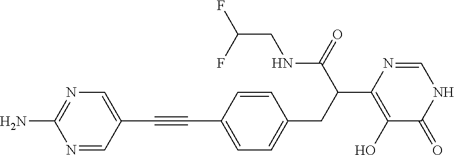 [SMILES: Nc1ncc(C#Cc2ccc(CC(C(=O)NCC(F)F)c3nc[nH]c(=O)c3O)cc2)cn1] | 441.35 |
| 25 | N-(2,2-difluoroethyl)-2-(5- hydroxy-6-oxo-1,6- dihydropyrimidin-4-yl)-3-(4- ((2-methylpyrimidin-5- yl)ethynyl)phenyl)propanamide | 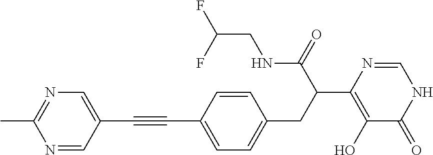 [SMILES: Cc1ncc(C#Cc2ccc(CC(C(=O)NCC(F)F)c3nc[nH]c(=O)c3O)cc2)cn1] | 440.12 |
| 26 | N-(2,2-difluoroethyl)-2-(5- hydroxy-6-oxo-1,6- dihydropyrimidin-4-yl)-3-(4- ((6-(trifluoromethyl)pyridin- 3-yl)ethynyl)phenyl) propanamide | 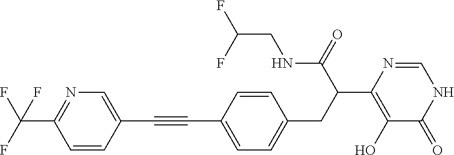 [SMILES: O=C(NCC(F)F)C(Cc1ccc(C#Cc2ccc(C(F)(F)F)nc2)cc1)c1nc[nH]c(=O)c1O] | 493.21 |
| 27 | N-(2,2-difluoroethyl)-2-(5- hydroxy-6-oxo-1,6- dihydropyrimidin-4-yl)-3-(4- ((2- (trifluoromethyl)pyrimidin-5- yl)ethynyl)phenyl) propanamide | 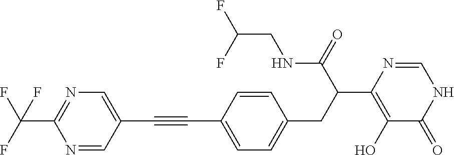 [SMILES: O=C(NCC(F)F)C(Cc1ccc(C#Cc2cnc(C(F)(F)F)nc2)cc1)c1nc[nH]c(=O)c1O] | 494.31 |
| 28 | 3-(4-((6-aminopyridin-3- yl)ethynyl)phenyl)-N-(2,2- difluoroethyl)-2-(5-hydroxy- 6-oxo-1,6-dihydropyrimidin- 4-yl)propanamide | 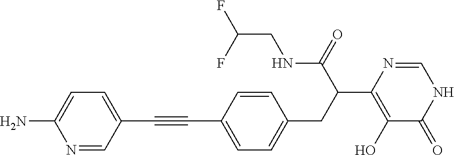 [SMILES: Nc1ccc(C#Cc2ccc(CC(C(=O)NCC(F)F)c3nc[nH]c(=O)c3O)cc2)cn1] | 440.45 |
| 29 | methyl (2-(5-hydroxy-6-oxo- 1,6-dihydropyrimidin-4-yl)- 3-(5-((4- (morpholinomethyl)phenyl) ethynyl)pyridin-2- yl)propyl)carbamate | 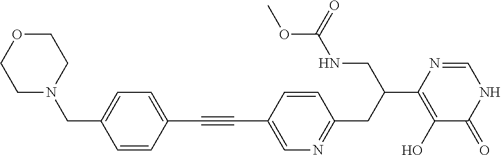 [SMILES: COC(=O)NCC(Cc1ccc(C#Cc2ccc(CN3CCOCC3)cc2)cn1)c1nc[nH]c(=O)c1O] | 504.26 |
| 30 | 6-(1-((2-fluoroethyl)amino)- 3-(4-((5- (morpholinomethyl)pyridin- 2-yl)ethynyl)phenyl)propan- 2-yl)-5-hydroxypyrimidin- 4(3H)-one | 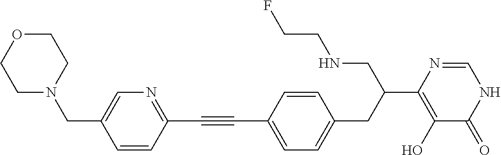 [SMILES: O=c1[nH]cnc(C(CNCCF)Cc2ccc(C#Cc3ccc(CN4CCOCC4)cn3)cc2)c1O] | 492.23 |
| 31 | 6-(1-((2-fluoroethyl)amino)- 3-(4-((6- (morpholinomethyl)pyridin- 3-yl)ethynyl)phenyl)propan- 2-yl)-5-hydroxypyrimidin- 4(3H)-one | 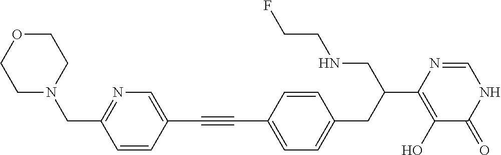 [SMILES: O=c1[nH]cnc(C(CNCCF)Cc2ccc(C#Cc3ccc(CN4CCOCC4)nc3)cc2)c1O] | 492.17 |
| 32 | 4-((4-(3-hydroxy-2-(5- hydroxy-6-oxo-1,6- dihydropyrimidin-4- yl)propyl)phenyl)buta-1,3- diyn-1-yl)-N-(2- hydroxyethyl)benzamide | 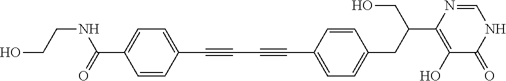 [SMILES: O=C(NCCO)c1ccc(C#CC#Cc2ccc(CC(CO)c3nc[nH]c(=O)c3O)cc2)cc1] | 458.19 |
| 33 | 5-hydroxy-6-(3-(4-((4-(4- methylpiperazine-1- carbonyl)phenyl)buta-1,3- diyn-1-yl)phenyl)prop-1-en- 2-yl)pyrimidin-4(3H)-one | 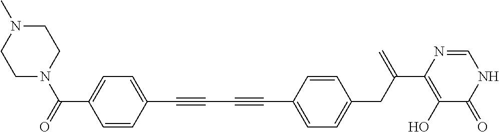 [SMILES: C=C(Cc1ccc(C#CC#Cc2ccc(C(=O)N3CCN(C)CC3)cc2)cc1)c1nc[nH]c(=O)c1O] | 479.25 |
| 34 | 4-((4-(3-(allylamino)-2-(5- hydroxy-6-oxo-1,6- dihydropyrimidin-4- yl)propyl)phenyl)buta-1,3- diyn-1-yl)-N-(2- hydroxyethyl)benzamide | 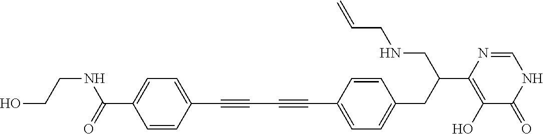 [SMILES: C=CCNCC(Cc1ccc(C#CC#Cc2ccc(C(=O)NCCO)cc2)cc1)c1nc[nH]c(=O)c1O] | 497.21 |
| 35 | 4-((4-(3-(allylamino)-2-(5 hydroxy-6-oxo-1,6- dihydropyrimidin-4- yl)propyl)phenyl)buta-1,3- diyn-1-yl)benzoic acid | 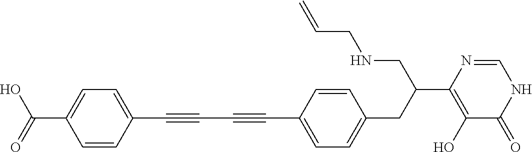 [SMILES: C=CCNCC(Cc1ccc(C#CC#Cc2ccc(C(=O)O)cc2)cc1)c1nc[nH]c(=O)c1O] | 454.10 |
| 36 | 6-(1-(allylamino)-3-(4-((2- aminophenyl)buta-1,3-diyn- 1-yl)phenyl)propan-2-yl)-5- hydroxypyrimidin-4(3H)-one | 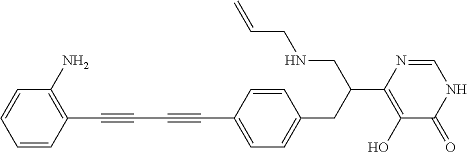 [SMILES: C=CCNCC(Cc1ccc(C#CC#Cc2ccccc2N)cc1)c1nc[nH]c(=O)c1O] | 425.20 |
| 37 | 6-(1-(allylamino)-3-(4-((2- methylpyrimidin-5-yl)buta- 1,3-diyn-1-yl)phenyl)propan- 2-yl)-5-hydroxypyrimidin- 4(3H)-one | 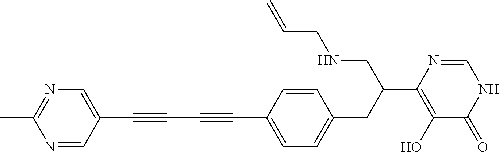 [SMILES: C=CCNCC(Cc1ccc(C#CC#Cc2cnc(C)nc2)cc1)c1nc[nH]c(=O)c1O] | 426.29 |
| 38 | 3-(4′-(1H-pyrazol-1-yl)-[1,1- biphenyl]-4-yl)-N-(2,2- difluoroethyl)-2-(5-hydroxy- 6-oxo-1,6-dihydropyrimidin- 4-yl)propanamide | 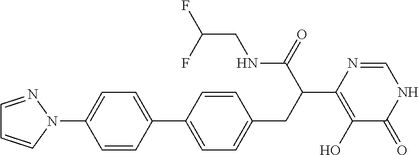 [SMILES: O=C(NCC(F)F)C(Cc1ccc(-c2ccc(-n3cccn3)cc2)cc1)c1nc[nH]c(=O)c1O] | 466.26 |
| 39 | 3-(4′-cyano-[1,1′-biphenyl]- 4-yl)-N-(2,2-difluoroethyl)-2- (5-hydroxy-6-oxo-1,6- dihydropyrimidin-4- yl)propanamide | 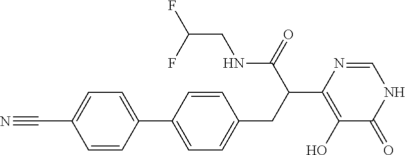 [SMILES: N#Cc1ccc(-c2ccc(CC(C(=O)NCC(F)F)c3nc[nH]c(=O)c3O)cc2)cc1] | 425.41 |
| 40 | 3-([1,1′-biphenyl]-4-yl)-2-(5- hydroxy-6-oxo-1,6- dihydropyrimidin-4- yl)propanoic acid | 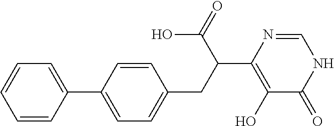 [SMILES: O=C(O)C(Cc1ccc(-c2ccccc2)cc1)c1nc[nH]c(=O)c1O] | 337.31 |
| 41 | 6-(2-([1,1′-biphenyl]-4- yl)ethyl)-5- hydroxypyrimidin-4(3H)-one | 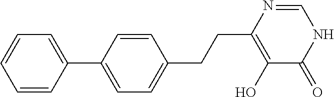 [SMILES: O=c1[nH]cnc(CCc2ccc(-c3ccccc3)cc2)c1O] | 293.24 |
| 42 | 1-(2-(5,6- dihydroxypyrimidin-4- yl)propyl)-4-((4- (morpholinomethyl)phenyl) ethynyl)pyridin-2(1H)-one | 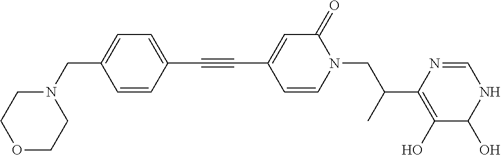 [SMILES: CC(Cn1ccc(C#Cc2ccc(CN3CCOCC3)cc2)cc1=O)C1=C(O)C(O)NC=N1] | |
| 43 | 5-hydroxy-6-(1-((4-((4- (morpholinomethyl)phenyl) ethynyl)-2-oxopyridin-1(2H)- yl)methyl)cyclopropyl) pyrimidin-4(3H)-one | 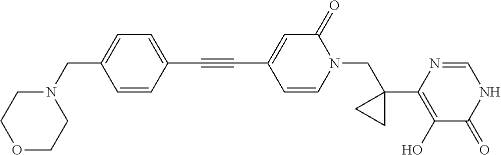 [SMILES: O=c1[nH]cnc(C2(Cn3ccc(C#Cc4ccc(CN5CCOCC5)cc4)cc3=O)CC2)c1O] | |
| 44 | 5-hydroxy-6-((4-((4- (morpholinomethyl)phenyl) ethynyl)-2-oxopyridin-1(2H)- yl)methyl)pyrimidin-4(3H)- one | 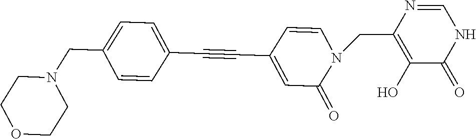 [SMILES: O=c1[nH]cnc(Cn2ccc(C#Cc3ccc(CN4CCOCC4)cc3)cc2=O)c1O] | |
| 45 | 5-hydroxy-6-(2-(2-oxo-4-((4- (((tetrahydrofuran-3- yl)amino)methyl)phenyl) ethynyl)pyridin-1(2H)- yl)ethyl)pyrimidin-4(3H)-one | 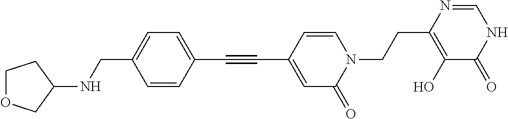 [SMILES: O=c1[nH]cnc(CCn2ccc(C#Cc3ccc(CNC4CCOC4)cc3)cc2=O)c1O] | |
| 46 | 5-hydroxy-6-(2-(4-((1- methyl-1H-pyrazol-4- yl)ethynyl)-2-oxopyridin- 1(2H)-yl)ethyl)pyrimidin- 4(3H)-one | 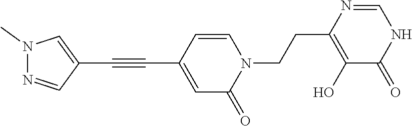 [SMILES: Cn1cc(C#Cc2ccn(CCc3nc[nH]c(=O)c3O)c(=O)c2)cn1] | |
| 47 | 6-(4-((5-chloropyridin-3- yl)ethynyl)phenethyl)-5- hydroxypyrimidin-4(3H)-one | 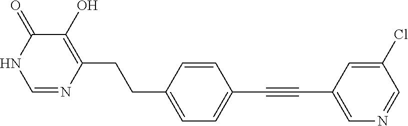 [SMILES: O=c1[nH]cnc(CCc2ccc(C#Cc3cncc(Cl)c3)cc2)c1O] | |
| 48 | 5-hydroxy-6-(4-((6- (((tetrahydrofuran-3- yl)amino)methyl)pyridin-3- yl)ethynyl)phenethyl) pyrimidin-4(3H)-one | 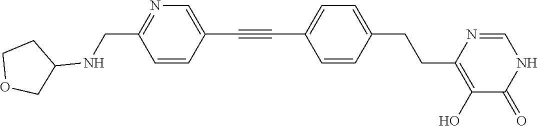 [SMILES: O=c1[nH]cnc(CCc2ccc(C#Cc3ccc(CNC4CCOC4)nc3)cc2)c1O] | |
| 49 | 5-hydroxy-6-(2-(4-(4- (morpholinomethyl)phenethyl)- 2-oxopyridin-1(2H)- yl)ethyl)pyrimidin-4(3H)-one | 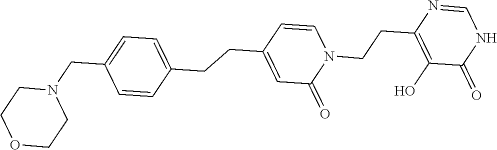 [SMILES: O=c1[nH]cnc(CCn2ccc(CCc3ccc(CN4CCOCC4)cc3)cc2=O)c1O] | |
| 50 | 6-(1-((2-fluoroethyl)amino)- 3-(4-(4- (morpholinomethyl)phenethyl) phenyl)propan-2-yl)-5- hydroxypyrimidin-4(3H)-one | 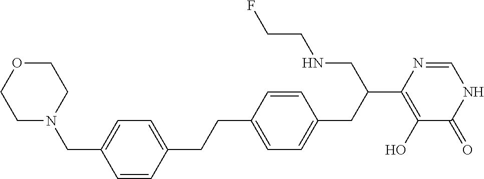 [SMILES: O=c1[nH]cnc(C(CNCCF)Cc2ccc(CCc3ccc(CN4CCOCC4)cc3)cc2)c1O] | |
| 51 | (E)-6-(1-((2- fluoroethyl)amino)-3-(4-(4- (morpholinomethyl)styryl) phenyl)propan-2-yl)-5- hydroxypyrimidin-4(3H)-one | 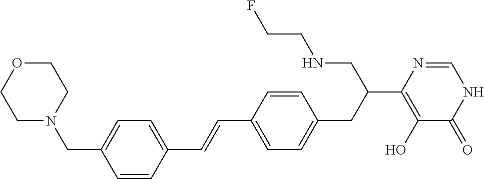 [SMILES: O=c1[nH]cnc(C(CNCCF)Cc2ccc(/C=C/c3ccc(CN4CCOCC4)cc3)cc2)c1O] | |
| 52 | (E)-6-(3-fluoro-4-(4- (morpholinomethyl)styryl) phenethyl)-5- hydroxypyrimidin-4(3H)-one | 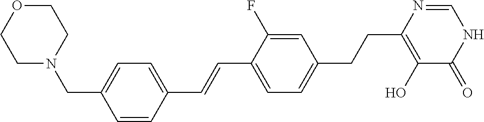 [SMILES: O=c1[nH]cnc(CCc2ccc(/C=C/c3ccc(CN4CCOCC4)cc3)c(F)c2)c1O] | |
| 53 | N-(2-(5-hydroxy-6-oxo-1,6- dihydropyrimidin-4-yl)-3-(4- ((4-(morpholinomethyl)phenyl) thio)phenyl)propyl)acetamide | 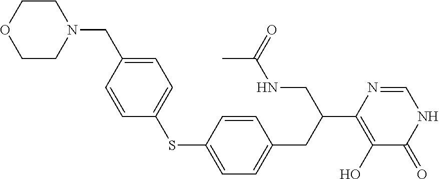 [SMILES: CC(=O)NCC(Cc1ccc(Sc2ccc(CN3CCOCC3)cc2)cc1)c1nc[nH]c(=O)c1O] | |
| 54 | (E)-N-(2,2-difluoroethyl)-2- (5-hydroxy-6-oxo-1,6- dihydropyrimidin-4-yl)-3-(4- (4-(morpholinomethyl)styryl) phenyl)propanamide | 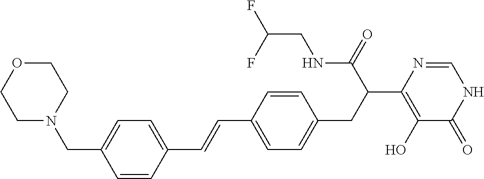 [SMILES: O=C(NCC(F)F)C(Cc1ccc(/C=C/c2ccc(CN3CCOCC3)cc2)cc1)c1nc[nH]c(=O)c1O] | |
| 55 | 6-(1-((2-fluoroethyl)amino)- 3-(4-((4- (morpholinomethyl)phenyl) thio)phenyl)propan-2-yl)-5- hydroxypyrimidin-4(3H)-one | 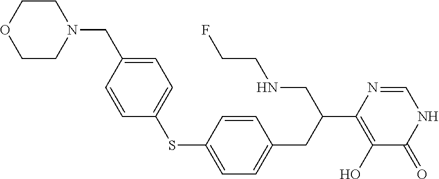 [SMILES: O=c1[nH]cnc(C(CNCCF)Cc2ccc(Sc3ccc(CN4CCOCC4)cc3)cc2)c1O] | |
| 56 | N-(2,2-difluoroethyl)-3-(4- ((4-(1,2- dihydroxyethyl)phenyl)ethynyl)- 2-oxopyridin-1(2H)-yl)- 2-(5-hydroxy-6-oxo-1,6- dihydropyrimidin-4- yl)propanamide | 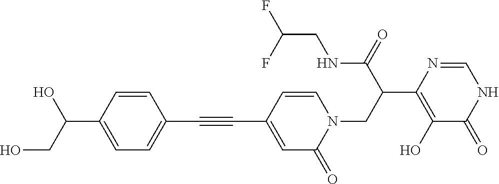 [SMILES: O=C(NCC(F)F)C(Cn1ccc(C#Cc2ccc(C(O)CO)cc2)cc1=O)c1nc[nH]c(=O)c1O] | |
| 57 | 6-(2-(5-((4-((2,2-dioxido-6- oxa-2-thia-9- azaspiro[4.5]decan-9- yl)methyl)phenyl)ethynyl) pyridin-2-yl)ethyl)-5- hydroxypyrimidin-4(3H)-one | 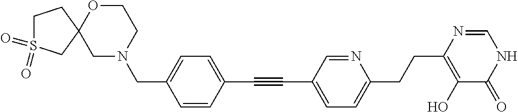 [SMILES: O=c1[nH]cnc(CCc2ccc(C#Cc3ccc(CN4CCOC5(CCS(=O)(=O)C5)C4)cc3)cn2)c1O] | |
| 58 | 6-(1-(ethylamino)-3-(4-(4- ((3-methoxyazetidin-1- yl)methyl)phenoxy)phenyl) propan-2-yl)-5- hydroxypyrimidin-4(3H)-one | 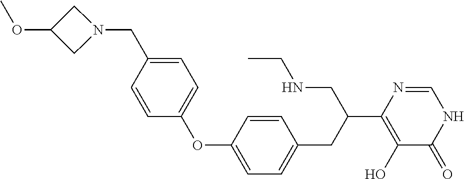 [SMILES: CCNCC(Cc1ccc(Oc2ccc(CN3CC(OC)C3)cc2)cc1)c1nc[nH]c(=O)c1O] | |
| 59 | 5-hydroxy-6-(2-(5-((4-(((5- (trifluoromethyl)-1,3,4- oxadiazol-2- yl)amino)methyl)phenyl) ethynyl)pyridin-2- yl)ethyl)pyrimidin-4(3H)-one | 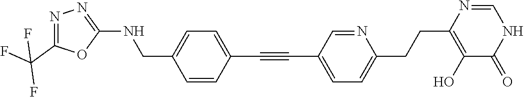 [SMILES: O=c1[nH]cnc(CCc2ccc(C#Cc3ccc(CNc4nnc(C(F)(F)F)o4)cc3)cn2)c1O] | |
| 60 | 5-hydroxy-6-(4-((5-((3- methoxypyrrolidin-1- yl)methyl)pyridin-2- yl)ethynyl)phenethyl) pyrimidin-4(3H)-one | 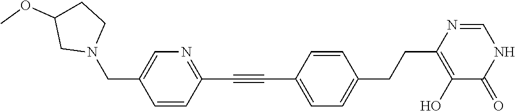 [SMILES: COC1CCN(Cc2ccc(C#Cc3ccc(CCc4nc[nH]c(=O)c4O)cc3)nc2)C1] | |
| 61 | 3-(4-(cyclopropylbuta-1,3- diyn-1-yl)-3-fluorophenyl)- N-(2,2-difluoroethyl)-2-(5- hydroxy-6-oxo-1,6- dihydropyrimidin-4- yl)propanamide | 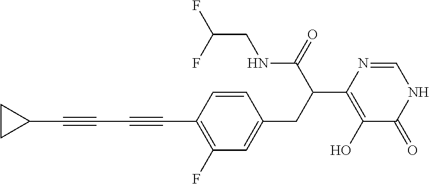 [SMILES: O=C(NCC(F)F)C(Cc1ccc(C#CC#CC2CC2)c(F)c1)c1nc[nH]c(=O)c1O] | 430.14 |
| 62 | 6-(1-(allylamino)-3-(4-((1- methyl-1H-pyrazol-4- yl)buta-1,3-diyn-1- yl)phenyl)propan-2-yl)-5- hydroxypyrimidin-4(3H)-one | 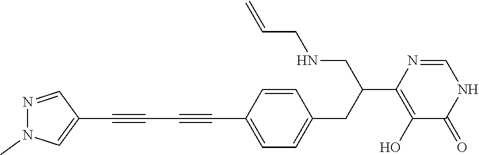 [SMILES: C=CCNCC(Cc1ccc(C#CC#Cc2cnn(C)c2)cc1)c1nc[nH]c(=O)c1O] | 414.20 |
| 63 | N-(2,2-difluoroethyl)-3-(3- fluoro-4-(5-methylhexa-1,3- diyn-1-yl)phenyl)-2-(5- hydroxy-6-oxo-1,6- dihydropyrimidin-4- yl)propanamide | 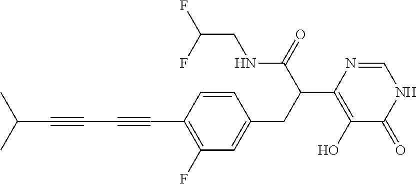 [SMILES: CC(C)C#CC#Cc1ccc(CC(C(=O)NCC(F)F)c2nc[nH]c(=O)c2O)cc1F] | 432.12 |
| 64 | N-(3-(4-(cyclopropylbuta- 1,3-diyn-1-yl)-3- fluorophenyl)-2-(5-hydroxy- 6-oxo-1,6-dihydropyrimidin- 4-yl)propyl)acetamide | 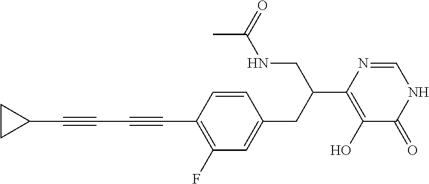 [SMILES: CC(=O)NCC(Cc1ccc(C#CC#CC2CC2)c(F)c1)c1nc[nH]c(=O)c1O] | 394.16 |
| 65 | N-(3-(4-(cyclopropylbuta- 1,3-diyn-1-yl)phenyl)-2-(5- hydroxy-6-oxo-1,6- dihydropyrimidin-4- yl)propyl)-2-fluoroacetamide | 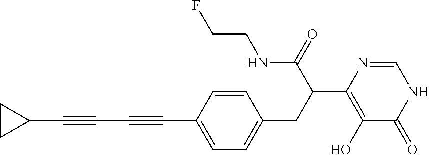 [SMILES: O=C(NCCF)C(Cc1ccc(C#CC#CC2CC2)cc1)c1nc[nH]c(=O)c1O] | 394.35 |
| 66 | 6-(1-((2-fluoroethyl)amino)- 3-(4-(5-methylhexa-1,3-diyn- 1-yl)phenyl)propan-2-yl)-5- hydroxypyrimidin-4(3H)-one | 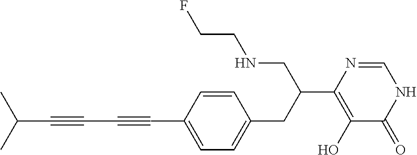 [SMILES: CC(C)C#CC#Cc1ccc(CC(CNCCF)c2nc[nH]c(=O)c2O)cc1] | 382.15 |
| 67 | 6-(1-(4-((1H-pyrazol-4- yl)buta-1,3-diyn-1- yl)phenyl)-3- (allylamino)propan-2-yl)-5- hydroxypyrimidin-4(3H)-one | 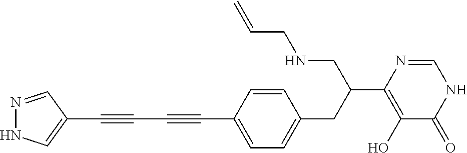 [SMILES: C=CCNCC(Cc1ccc(C#CC#Cc2cn[nH]c2)cc1)c1nc[nH]c(=O)c1O] | 400.18 |
| 68 | 6-(1-(4-(cyclopropylbuta-1,3- diyn-1-yl)phenyl)-3-((2- fluoroethyl)amino)propan-2- yl)-5-hydroxypyrimidin- 4(3H)-one | 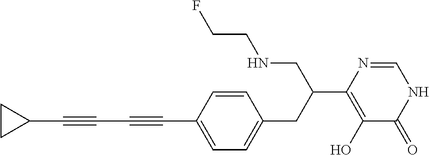 [SMILES: O=c1[nH]cnc(C(CNCCF)Cc2ccc(C#CC#CC3CC3)cc2)c1O] | 380.14 |
| 69 | 6-bromo-2-(2-(5-hydroxy-6- oxo-1,6-dihydropyrimidin-4- yl)ethyl)-1,2-dihydro-3H- pyrrolo[1,2-c]imidazol-3-one | 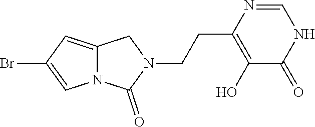 [SMILES: O=C1N(CCc2nc[nH]c(=O)c2O)Cc2cc(Br)cn21] | 340.05 |
| 70 | 2-(4-(2-(5-hydroxy-6-oxo- 1,6-dihydropyrimidin-4- yl)ethyl)phenyl)benzofuran- 5-carbonitrile | 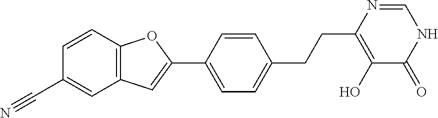 [SMILES: N#Cc1ccc2oc(-c3ccc(CCc4nc[nH]c(=O)c4O)cc3)cc2c1] | 358.17 |
| 71 | 6-(1-((2-fluoroethyl)amino)- 3-(4-((1-methyl-1H-pyrazol- 4-yl)ethynyl)phenyl)propan- 2-yl)-5-hydroxypyrimidin- 4(3H)-one | 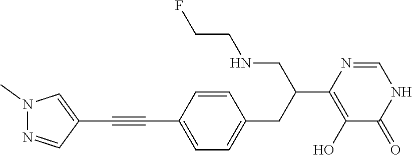 [SMILES: Cn1cc(C#Cc2ccc(CC(CNCCF)c3nc[nH]c(=O)c3O)cc2)cn1] | 396.41 |
| 72 | 6-(1-((2-fluoroethyl)amino)- 3-(4-((1-((tetrahydro-2H- pyran-4-yl)methyl)-1H- pyrazol-4- yl)ethynyl)phenyl)propan-2- yl)-5-hydroxypyrimidin- 4(3H)-one | 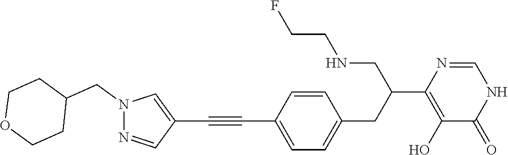 [SMILES: O=c1[nH]cnc(C(CNCCF)Cc2ccc(C#Cc3cnn(CC4CCOCC4)c3)cc2)c1O] | 480.14 |
| 73 | 6-((4-(3-((2,2- difluoroethyl)amino)-2-(5- hydroxy-6-oxo-1,6- dihydropyrimidin-4- yl)propyl)phenyl)ethynyl)-2- methylisoindolin-1-one | 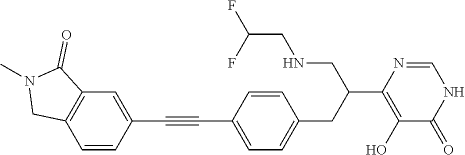 [SMILES: CN1Cc2ccc(C#Cc3ccc(CC(CNCC(F)F)c4nc[nH]c(=O)c4O)cc3)cc2C1=O] | 479.15 |
| 74 | 3-(4-((2-acetylisoindolin-5- yl)ethynyl)phenyl)-N-(2,2- difluoroethyl)-2-(5-hydroxy- 6-oxo-1,6-dihydropyrimidin- 4-yl)propanamide | 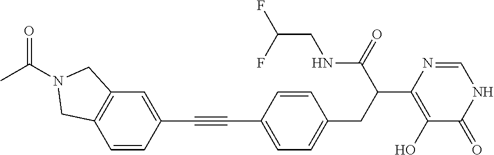 [SMILES: CC(=O)N1Cc2ccc(C#Cc3ccc(CC(C(=O)NCC(F)F)c4nc[nH]c(=O)c4O)cc3)cc2C1] | 507.31 |
| 75 | N-(2,2-difluoroethyl)-2-(5- hydroxy-6-oxo-1,6- dihydropyrimidin-4-yl)-3-(4- ((1-(2-hydroxyethyl)-1H- pyrazol-4- yl)ethynyl)phenyl) propanamide | 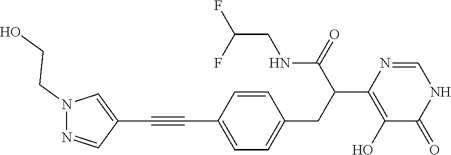 [SMILES: O=C(NCC(F)F)C(Cc1ccc(C#Cc2cnn(CCO)c2)cc1)c1nc[nH]c(=O)c1O] | 458.14 |
| 76 | N-(2,2-difluoroethyl)-2-(5- hydroxy-6-oxo-1,6- dihydropyrimidin-4-yl)-3-(4- ((1-methyl-1H-pyrazol-4- yl)ethynyl)phenyl)propanamide | 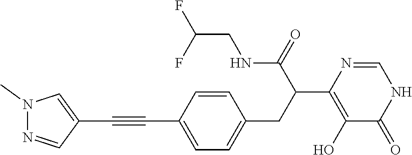 [SMILES: Cn1cc(C#Cc2ccc(CC(C(=O)NCC(F)F)c3nc[nH]c(=O)c3O)cc2)cn1] | 428.31 |
| 77 | 5-hydroxy-6-(2-(4-((4- (morpholinomethyl)phenyl) ethynyl)piperidin-1-yl)-2- oxoethyl)pyrimidin-4(3H)- one | 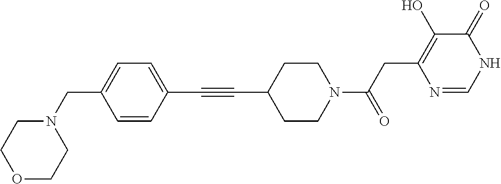 [SMILES: O=C(Cc1nc[nH]c(=O)c1O)N1CCC(C#Cc2ccc(CN3CCOCC3)cc2)CC1] | |
| 78 | 5-hydroxy-6-(2-(4-((4- (morpholinomethyl)phenyl) ethynyl)piperidin-1- yl)ethyl)pyrimidin-4(3H)-one | 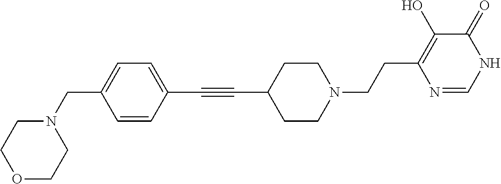 [SMILES: O=c1[nH]cnc(CCN2CCC(C#Cc3ccc(CN4CCOCC4)cc3)CC2)c1O] | |
| 79 | N-(2-(5-hydroxy-6-oxo-1,6- dihydropyrimidin-4- yl)ethyl)-2-(hydroxymethyl)- 4-((4- (morpholinomethyl)phenyl) ethynyl)-1H-pyrrole-1- carboxamide | 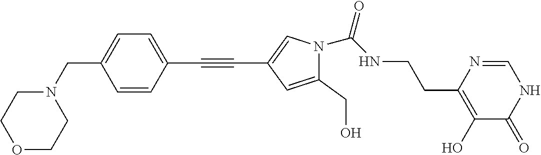 [SMILES: O=C(NCCc1nc[nH]c(=O)c1O)n1cc(C#Cc2ccc(CN3CCOCC3)cc2)cc1CO] | |
| 80 | 5-hydroxy-6-((5-((4- (morpholinomethyl)phenyl) ethynyl)-1H-pyrrolo[2,3- b]pyridin-1- yl)methyl)pyrimidin-4(3H)- one | 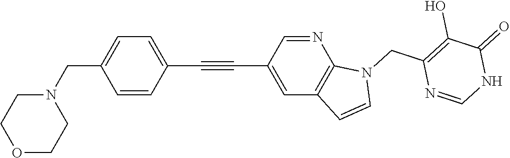 [SMILES: O=c1[nH]cnc(Cn2ccc3cc(C#Cc4ccc(CN5CCOCC5)cc4)cnc32)c1O] | |
| 81 | 2-((5-hydroxy-6-oxo-1,6- dihydropyrimidin-4- yl)methyl)-6-((4- (morpholinomethyl)phenyl) ethynyl)-1,2-dihydro-3H- pyrrolo[1,2-c]imidazol-3-one | 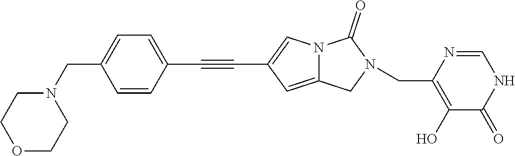 [SMILES: O=C1N(Cc2nc[nH]c(=O)c2O)Cc2cc(C#Cc3ccc(CN4CCOCC4)cc3)cn21] | |
| 82 | 2-(2-(5-hydroxy-6-oxo-1,6- dihydropyrimidin-4- yl)ethyl)-6-((4- (morpholinomethyl)phenyl) ethynyl)-1,2-dihydro-3H- pyrrolo[1,2-c]imidazol-3-one | 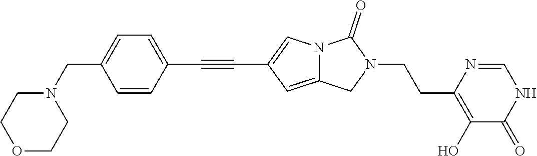 [SMILES: O=C1N(CCc2nc[nH]c(=O)c2O)Cc2cc(C#Cc3ccc(CN4CCOCC4)cc3)cn21] | |
| 83 | 5-hydroxy-6-(4-((1- ((tetrahydro-2H-pyran-4- yl)methyl)-1H-pyrazol-4- yl)ethynyl)phenethyl) pyrimidin-4(3H)-one | 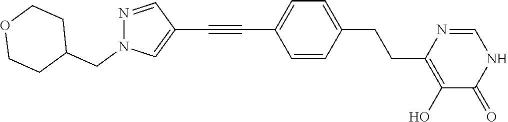 [SMILES: O=c1[nH]cnc(CCc2ccc(C#Cc3cnn(CC4CCOCC4)c3)cc2)c1O] | |
| 84 | 5-hydroxy-6-(4-((1-methyl- 1H-pyrazol-4- yl)ethynyl)phenethyl)pyrimidin- 4(3H)-one | 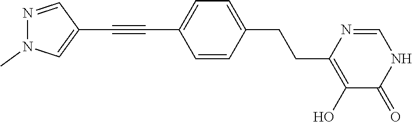 [SMILES: Cn1cc(C#Cc2ccc(CCc3nc[nH]c(=O)c3O)cc2)cn1] | |
| 85 | 2-(2-(5-hydroxy-6-oxo-1,6- dihydropyrimidin-4- yl)ethyl)-6-((1-methyl-1H- pyrazol-4-yl)ethynyl)-1,2- dihydro-3H-pyrrolo[1,2- c]imidazol-3-one | 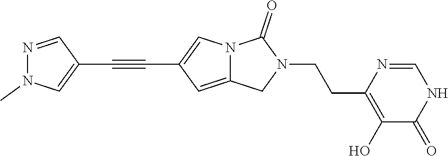 [SMILES: Cn1cc(C#Cc2cc3n(c2)C(=O)N(CCc2nc[nH]c(=O)c2O)C3)cn1] | |
| 86 | 5-hydroxy-6-(4-(piperidin-4- ylethynyl)phenethyl)pyrimidin- 4(3H)-one | 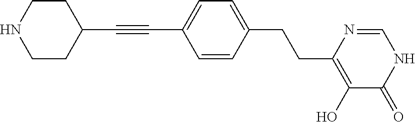 [SMILES: O=c1[nH]cnc(CCc2ccc(C#CC3CCNCC3)cc2)c1O] | |
| 87 | 5-hydroxy-6-(4-((1- methylpiperidin-4- yl)ethynyl)phenethyl) pyrimidin-4(3H)-one | 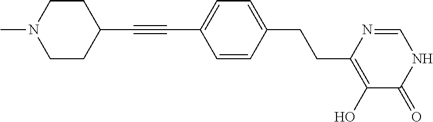 [SMILES: CN1CCC(C#Cc2ccc(CCc3nc[nH]c(=O)c3O)cc2)CC1] | |
| 88 | 6-(2-(4-(2-fluoro-4- methoxyphenyl)-2- oxopyridin-1(2H)-yl)ethyl)- 5-hydroxypyrimidin-4(3H)- one | 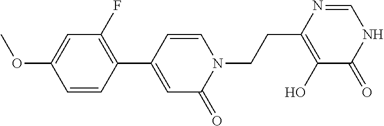 [SMILES: COc1ccc(-c2ccn(CCc3nc[nH]c(=O)c3O)c(=O)c2)c(F)c1] | |
| 89 | 2-(2-(5-hydroxy-6-oxo-1,6- dihydropyrimidin-4- yl)ethyl)-6-(1-methyl-1H- pyrazol-4-yl)isoquinolin- 1(2H)-one | 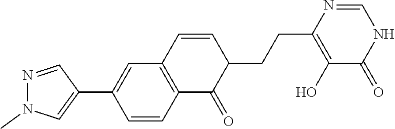 [SMILES: Cn1cc(-c2ccc3c(c2)C=CC(CCc2nc[nH]c(=O)c2O)C3=O)cn1] | |
| 90 | 6-(1-(6- (cyclopropylethynyl)imidazo [1,2-a]pyridin-2-yl)-3-((2- fluoroethyl)amino)propan-2- yl)-5-hydroxypyrimidin- 4(3H)-one | 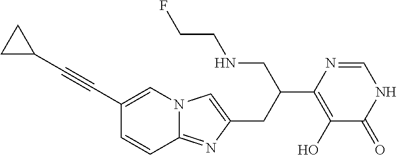 [SMILES: O=c1[nH]cnc(C(CNCCF)Cc2cn3cc(C#CC4CC4)ccc3n2)c1O] | |
| 91 | 6-(1-(5-(cyclopropylbuta-1,3- diyn-1-yl)-1H-indazol-1-yl)- 3-((2- fluoroethyl)amino)propan-2- yl)-5-hydroxypyrimidin- 4(3H)-one | 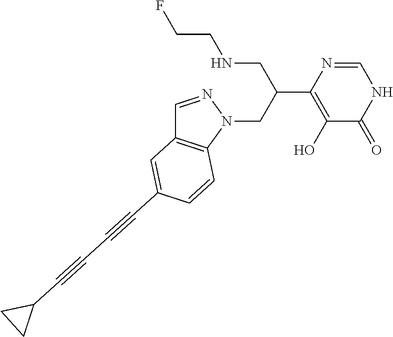 [SMILES: O=c1[nH]cnc(C(CNCCF)Cn2ncc3cc(C#CC#CC4CC4)ccc32)c1O] | |
| 92 | 5-(cyclopropylethynyl)-2-(3- ((2-fluoroethyl)amino)-2-(5- hydroxy-6-oxo-1,6- dihydropyrimidin-4- yl)propyl)isoindolin-1-one | 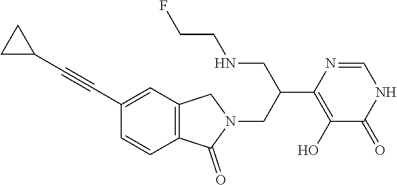 [SMILES: O=C1c2ccc(C#CC3CC3)cc2CN1CC(CNCCF)c1nc[nH]c(=O)c1O] | |
| 93 | 6-(1-(6- (cyclopropylethynyl)indolizin- 2-yl)-3-((2- fluoroethyl)amino)propan-2- yl)-5-hydroxypyrimidin- 4(3H)-one | 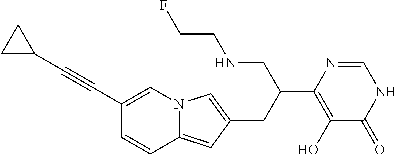 [SMILES: O=c1[nH]cnc(C(CNCCF)Cc2cc3ccc(C#CC4CC4)cn3c2)c1O] | |
| 94 | 5-hydroxy-6-(6- (phenylethynyl)-2,3- dihydrobenzofuran-2- yl)pyrimidin-4(3H)-one | 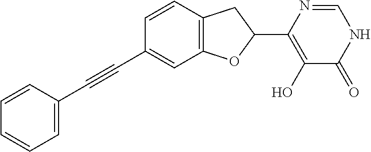 [SMILES: O=c1[nH]cnc(C2Cc3ccc(C#Cc4ccccc4)cc3O2)c1O] | |
| 95 | 5-(6-(3-((2- fluoroethyl)amino)-2-(5- hydroxy-6-oxo-1,6- dihydropyrimidin-4- yl)propyl)pyridin-3- yl)isoindolin-1-one | 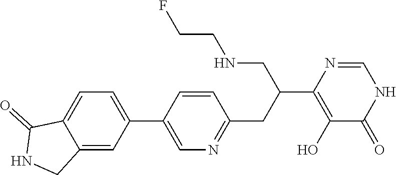 [SMILES: O=C1NCc2cc(-c3ccc(CC(CNCCF)c4nc[nH]c(=O)c4O)nc3)ccc21] | |
| 96 | (E)-3-(2-((2- fluoroethyl)amino)-1-(5- hydroxy-6-oxo-1,6- dihydropyrimidin-4- yl)ethyl)-6-(4- (morpholinomethyl)styryl) isoindolin-1-one | 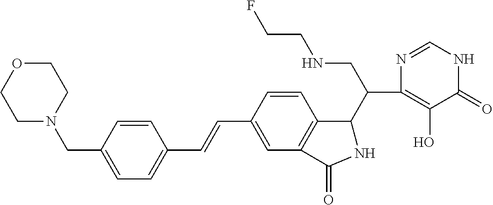 [SMILES: O=C1NC(C(CNCCF)c2nc[nH]c(=O)c2O)c2ccc(/C=C/c3ccc(CN4CCOCC4)cc3)cc21] | |
| 97 | 6-(1-(4-([1,3]dioxolo[4,5- f]benzofuran-6-yl)phenyl)-3- ((2- fluoroethyl)amino)propan-2- yl)-5-hydroxypyrimidin- 4(3H)-one | 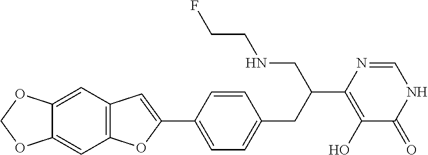 [SMILES: O=c1[nH]cnc(C(CNCCF)Cc2ccc(-c3cc4cc5c(cc4o3)OCO5)cc2)c1O] | |
| 98 | 6-(1-((4-(benzofuran-2- yl)phenyl)thio)-2-((2- fluoroethyl)amino)ethyl)-5- hydroxypyrimidin-4(3H)-one | 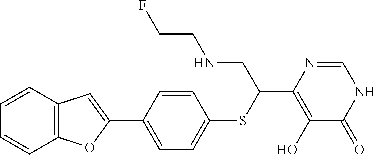 [SMILES: O=c1[nH]cnc(C(CNCCF)Sc2ccc(-c3cc4ccccc4o3)cc2)c1O] | |
| 99 | N -(2-fluoroethyl)-2-(5- hydroxy-6-oxo-1,6- dihydropyrimidin-4-yl)-3-(6- (4-(2-hydroxyethyl)phenyl)- 3-oxo-1H-pyrrolo[1,2- c]imidazol-2(3H)- yl)propanamide | 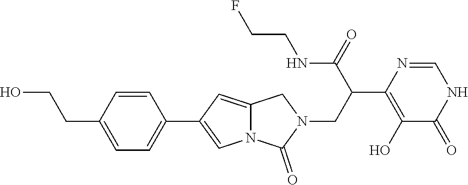 [SMILES: O=C(NCCF)C(CN1Cc2cc(-c3ccc(CCO)cc3)cn2C1=O)c1nc[nH]c(=O)c1O] | |
| 100 | 6-(1-(5-(cyclopropylethynyl)- 2H-indazol-2-yl)-3-((2- fluoroethyl)amino)propan-2- yl)-5-hydroxypyrimidin- 4(3H)-one | 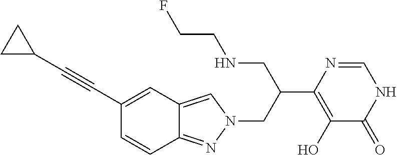 [SMILES: O=c1[nH]cnc(C(CNCCF)Cn2cc3cc(C#CC4CC4)ccc3n2)c1O] | |
| 101 | 3-((S)-3-hydroxy-2-(4-((4- ((((S)-tetrahydrofuran-3- yl)amino)methyl)phenyl)ethynyl) phenyl)propyl)pyridazine-4,5-diol | 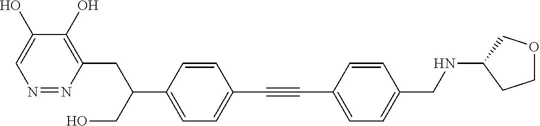 [SMILES: OCC(Cc1nncc(O)c1O)c1ccc(C#Cc2ccc(CN[C@H]3CCOC3)cc2)cc1] Single enantiomer (1^{st} eluting) | |
| 102 | 3-((S)-3-hydroxy-2-(4-((4- ((((S)-tetrahydrofuran-3- yl)amino)methyl)phenyl)ethynyl) phenyl)propyl)pyridazine-4,5-diol | 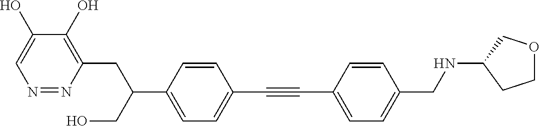 [SMILES: OCC(Cc1nncc(O)c1O)c1ccc(C#Cc2ccc(CN[C@H]3CCOC3)cc2)cc1] Single enantiomer (2^{nd} eluting) | |
| 103 | 5-hydroxy-6-(2-(4-((4-((((R)- pyrrolidin-3- yl)amino)methyl)phenyl) ethynyl)phenyl)cyclopropyl) pyrimidin-4(3H)-one | 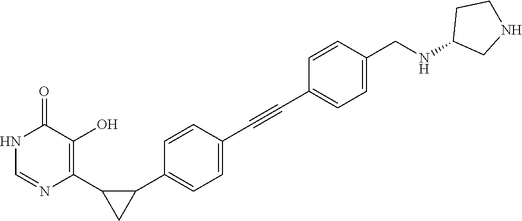 [SMILES: O=c1[nH]cnc(C2CC2c2ccc(C#Cc3ccc(CN[C@@H]4CCNC4)cc3)cc2)c1O] | |
| 104 | 5-hydroxy-6-(1-(4-((4- (morpholinomethyl)phenyl) ethynyl)phenyl)propan-2- yl)pyrimidin-4(3H)-one | 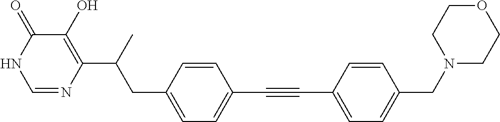 [SMILES: CC(Cc1ccc(C#Cc2ccc(CN3CCOCC3)cc2)cc1)c1nc[nH]c(=O)c1O] | |
| 105 | 5-hydroxy-6-(3-(4-((4- (morpholinomethyl)phenyl) ethynyl)phenyl)prop-1-en-2- yl)pyrimidin-4(3H)-one | 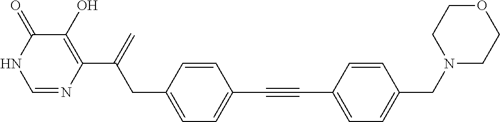 [SMILES: C=C(Cc1ccc(C#Cc2ccc(CN3CCOCC3)cc2)cc1)c1nc[nH]c(=O)c1O] | |
| 106 | 3-(5-hydroxy-6-oxo-1,6- dihydropyrimidin-4-yl)-4-(4- ((4- (morpholinomethyl)phenyl) ethynyl)phenyl)butanenitrile | 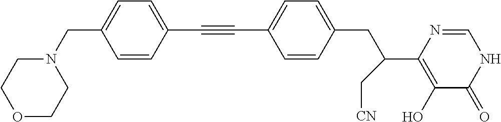 [SMILES: N#CCC(Cc1ccc(C#Cc2ccc(CN3CCOCC3)cc2)cc1)c1nc[nH]c(=O)c1O] | |
| 107 | 3-(5-hydroxy-6-oxo-1,6- dihydropyrimidin-4-yl)-4-(4- ((4-((((S)-tetrahydrofuran-3- yl)amino)methyl)phenyl) ethynyl)phenyl)butanenitrile | 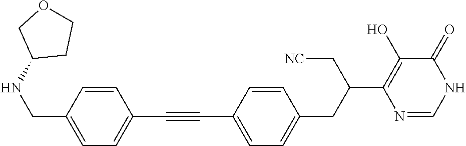 [SMILES: N#CCC(Cc1ccc(C#Cc2ccc(CN[C@H]3CCOC3)cc2)cc1)c1nc[nH]c(=O)c1O] | |
| 108 | 5-hydroxy-6-(((4-((4- (morpholinomethyl)phenyl) ethynyl)phenyl)amino)methyl) pyrimidin-4(3H)-one | 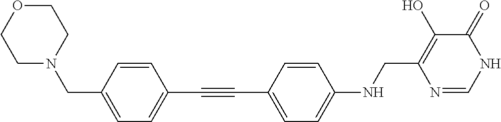 [SMILES: O=c1[nH]cnc(CNc2ccc(C#Cc3ccc(CN4CCOCC4)cc3)cc2)c1O] | |
| 109 | 3-hydroxy-2-(((4-((4- (morpholinomethyl)phenyl) ethynyl)phenyl)amino)methyl)- 4H-pyran-4-one | 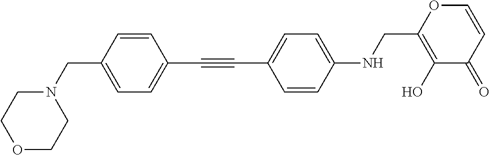 [SMILES: O=c1ccoc(CNc2ccc(C#Cc3ccc(CN4CCOCC4)cc3)cc2)c1O] | |
| 110 | 2-(3,4-dihydroxypyridin-2- yl)-N-methyl-3-(4-((4- (morpholinomethyl)phenyl) ethynyl)phenyl)propanamide | 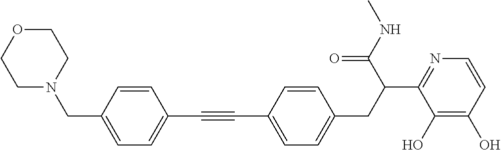 [SMILES: CNC(=O)C(Cc1ccc(C#Cc2ccc(CN3CCOCC3)cc2)cc1)c1nccc(O)c1O] | |
| 111 | (E)-3-(5,6- dihydroxypyrimidin-4-yl)-4- (4-((4- (morpholinomethyl)phenyl) ethynyl)phenyl)butan-2-one oxime | 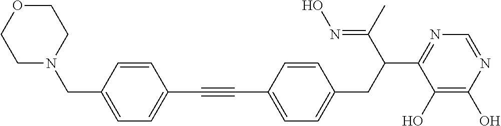 [SMILES: C/C(=N\O)C(Cc1ccc(C#Cc2ccc(CN3CCOCC3)cc2)cc1)c1ncnc(O)c1O] | |
| 112 | N-(2-(4,5- dihydroxypyridazin-3-yl)-3- (4-((4- (morpholinomethyl)phenyl) ethynyl)phenyl)propyl)acetamide | 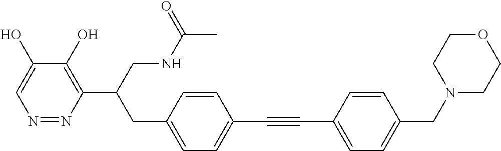 [SMILES: CC(=O)NCC(Cc1ccc(C#Cc2ccc(CN3CCOCC3)cc2)cc1)c1nncc(O)c1O] | |
| 113 | 6-(1-chloro-3-(4-((4- (morpholinomethyl)phenyl) ethynyl)phenyl)propan-2-yl)-5- hydroxypyrimidin-4(3H)-one | 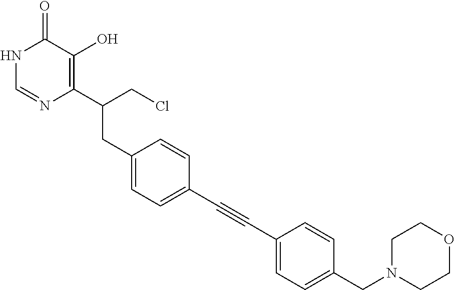 [SMILES: O=c1[nH]cnc(C(CCl)Cc2ccc(C#Cc3ccc(CN4CCOCC4)cc3)cc2)c1O] | |
| 114 | 3-hydroxy-1-methyl-2-(((4- ((4-(morpholinomethyl)phenyl) ethynyl)phenyl)amino)methyl) pyridin-4(1H)-one | 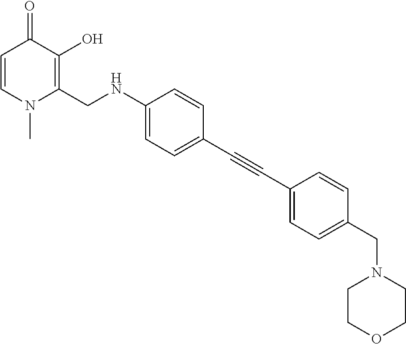 [SMILES: Cn1ccc(=O)c(O)c1CNc1ccc(C#Cc2ccc(CN3CCOCC3)cc2)cc1] | |
| 115 | tetrahydrothiophen-3-yl 2-(5- hydroxy-6-oxo-1,6- dihydropyrimidin-4-yl)-3-(4-((4- (morpholinomethyl)phenyl) ethynyl)phenyl)propanoate | 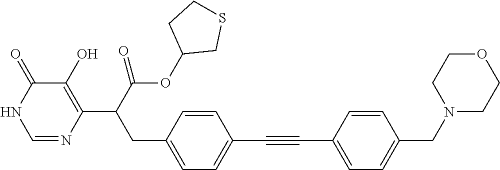 [SMILES: O=C(OC1CCSC1)C(Cc1ccc(C#Cc2ccc(CN3CCOCC3)cc2)cc1)c1nc[nH]c(=O)c1O] | |
| 116 | (E)-3-(5,6- dihydroxypyrimidin-4-yl)-4- (4-((4- (morpholinomethyl)phenyl) ethynyl)phenyl)butan-2-one O- methyl oxime | 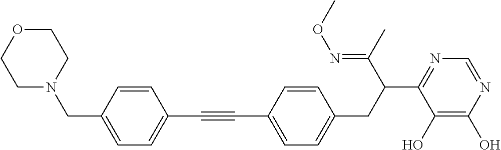 [SMILES: CO/N=C(\C)C(Cc1ccc(C#Cc2ccc(CN3CCOCC3)cc2)cc1)c1ncnc(O)c1O] | |
| 117 | 3-hydroxy-2-(4-((4- (morpholinomethyl)phenyl) ethynyl)phenethyl)-4H-pyran- 4-one | 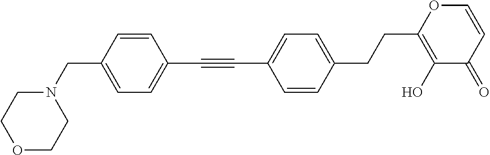 [SMILES: O=c1ccoc(CCc2ccc(C#Cc3ccc(CN4CCOCC4)cc3)cc2)c1O] | |
| 118 | 5-hydroxy-6-(((4-((4- (morpholinomethyl)phenyl) ethynyl)phenyl)sulfonyl)methyl) pyrimidin-4(3H)-one | 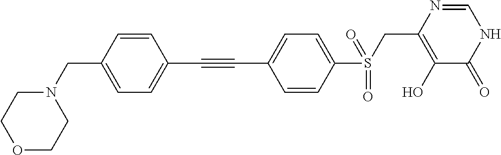 [SMILES: O=c1[nH]cnc(CS(=O)(=O)c2ccc(C#Cc3ccc(CN4CCOCC4)cc3)cc2)c1O] | |
| 119 | 5-hydroxy-6-(1-(4-((4- (morpholinomethyl)phenyl) ethynyl)phenyl)-3-(1H- tetrazol-1-yl)propan-2- yl)pyrimidin-4(3H)-one | 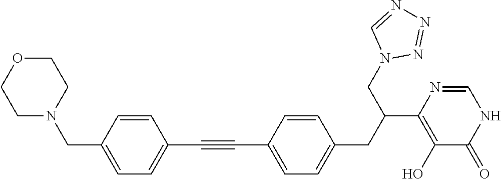 [SMILES: O=c1[nH]cnc(C(Cc2ccc(C#Cc3ccc(CN4CCOCC4)cc3)cc2)Cn2cnnn2)c1O] | |
| 120 | 3-hydroxy-2-(l1- (methylsulfonyl)-2-(4-((4- (morpholinomethyl)phenyl) ethynyl)phenyl)ethyl)-4H- pyran-4-one | 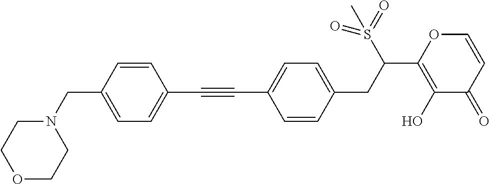 [SMILES: CS(=O)(=O)C(Cc1ccc(C#Cc2ccc(CN3CCOCC3)cc2)cc1)c1occc(=O)c1O] | |
| 121 | 2-(3-hydroxy-2-oxo-1,2- dihydropyridin-4-yl)-3-(4-((4- (morpholinomethyl)phenyl) ethynyl)phenyl)propanenitrile | 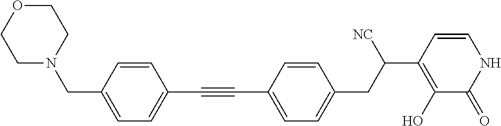 [SMILES: N#CC(Cc1ccc(C#Cc2ccc(CN3CCOCC3)cc2)cc1)c1cc[nH]c(=O)c1O] | |
| 122 | 4-((4-(((3-hydroxy-4-oxo- 4H-pyran-2- yl)methyl)amino)phenyl) ethynyl)benzonitrile | 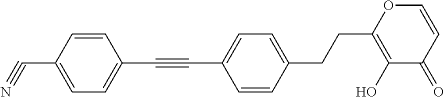 [SMILES: N#Cc1ccc(C#Cc2ccc(CCc3occc(=O)c3O)cc2)cc1] | |
| 123 | 4-((4-(((3-hydroxy-4-oxo- 4H-pyran-2- yl)methyl)amino)phenyl) ethynyl)benzoic acid | 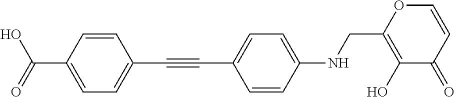 [SMILES: O=C(O)c1ccc(C#Cc2ccc(NCc3occc(=O)c3O)cc2)cc1] | |
| 124 | 3-hydroxy-2-(((4-((4- (morpholinomethyl)phenyl) ethynyl)phenyl)sulfonyl) methyl)-4H-pyran-4-one | 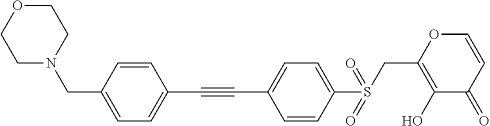 [SMILES: O=c1ccoc(CS(=O)(=O)c2ccc(C#Cc3ccc(CN4CCOCC4)cc3)cc2)c1O] | |
| 125 | N-(2-(3-hydroxy-2-oxo-1,2- dihydropyridin-4-yl)-3-(4- ((4-(morpholinomethyl)phenyl) ethynyl)phenyl)propyl)acetamide | 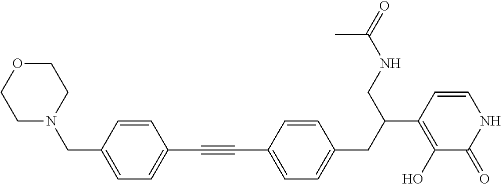 [SMILES: CC(=O)NCC(Cc1ccc(C#Cc2ccc(CN3CCOCC3)cc2)cc1)c1cc[nH]c(=O)c1O] | |
| 126 | 3-hydroxy-2-(1-((4-((4- (morpholinomethyl)phenyl) ethynyl)phenyl)amino)but-3- en-1-yl)-4H-pyran-4-one | 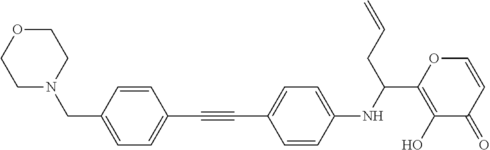 [SMILES: C=CCC(Nc1ccc(C#Cc2ccc(CN3CCOCC3)cc2)cc1)c1occc(=O)c1O] | |
| 127 | 5-hydroxy-6-(((2- hydroxyethyl)(4-((4- (morpholinomethyl)phenyl) ethynyl)phenyl)amino)methyl) pyrimidin-4(3H)-one | 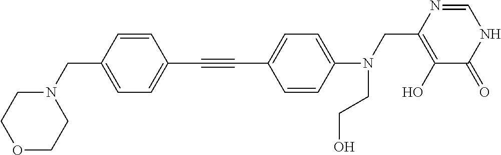 [SMILES: O=c1[nH]cnc(CN(CCO)c2ccc(C#Cc3ccc(CN4CCOCC4)cc3)cc2)c1O] | |
| 128 | 5-hydroxy-6-(((4-((4- (oxazol-2- yl)phenyl)ethynyl)phenyl) amino)methyl)pyrimidin-4(3H)- one | 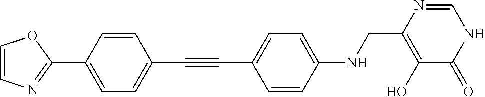 [SMILES: O=c1[nH]cnc(CNc2ccc(C#Cc3ccc(-c4ncco4)cc3)cc2)c1O] | |
| 129 | 6-(((4-((4-(1H-tetrazol-5- yl)phenyl)ethynyl)phenyl) amino)methyl)-5- hydroxypyrimidin-4(3H)-one | 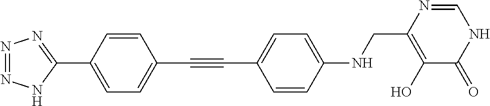 [SMILES: O=c1[nH]cnc(CNc2ccc(C#Cc3ccc(-c4nnn[nH]4)cc3)cc2)c1O] | |
| 130 | 3-hydroxy-4-oxo-N-(4- (phenylbuta-1,3-diyn-1- yl)phenyl)-4H-pyran-2- carboxamide | 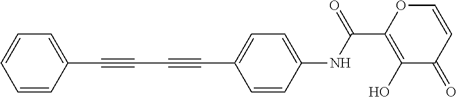 [SMILES: O=C(Nc1ccc(C#CC#Cc2ccccc2)cc1)c1occc(=O)c1O] | |
| 131 | N-(2-(5-hydroxy-3-oxo-2,3- dihydropyridazin-4-yl)-3-(4- ((4-(morpholinomethyl)phenyl) ethynyl)phenyl)propyl)acetamide | 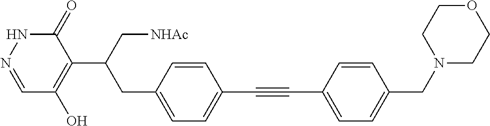 [SMILES: CC(=O)NCC(Cc1ccc(C#Cc2ccc(CN3CCOCC3)cc2)cc1)c1c(O)cn[nH]c1=O] | |
| 132 | 2-fluoro-N-(2-(5-hydroxy-1- methyl-6-oxo-1,6- dihydropyrimidin-4-yl)-3-(4- ((4-(morpholinomethyl)phenyl) ethynyl)phenyl)propyl)acetamide | 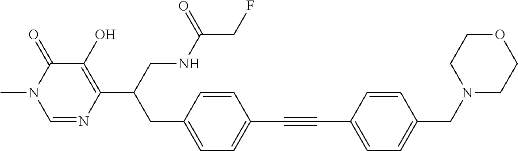 [SMILES: Cn1cnc(C(CNC(=O)CF)Cc2ccc(C#Cc3ccc(CN4CCOCC4)cc3)cc2)c(O)c1=O] | |
| 133 | 5-chloro-6-(4-((4- (morpholinomethyl)phenyl) ethynyl)phenethyl)pyrimidin- 4(3H)-one | 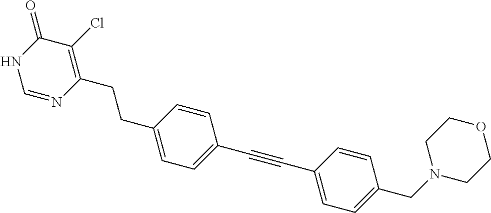 [SMILES: O=c1[nH]cnc(CCc2ccc(C#Cc3ccc(CN4CCOCC4)cc3)cc2)c1Cl] | |
| 134 | 2-(3-hydroxy-2-(4-((4- (morpholinomethyl)phenyl) ethynyl)phenyl)propyl)pyridine- 3,4-diol | 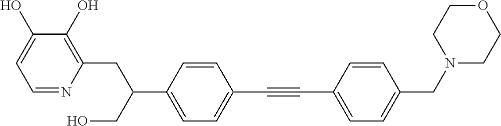 [SMILES: OCC(Cc1nccc(O)c1O)c1ccc(C#Cc2ccc(CN3CCOCC3)cc2)cc1] | |
| 135 | 5-hydroxy-6-(1-(4-((4- (morpholinomethyl)phenyl) ethynyl)benzyl)cyclopropyl) pyrimidin-4(3H)-one | 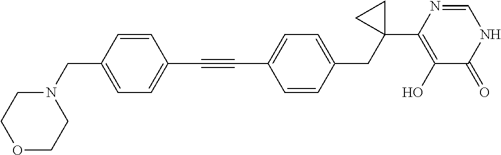 [SMILES: O=c1[nH]cnc(C2(Cc3ccc(C#Cc4ccc(CN5CCOCC5)cc4)cc3)CC2)c1O] | |
| 136 | 5-hydroxy-6-(1-(4-((4- (((tetrahydrofuran-3- yl)amino)methyl)phenyl)ethynyl) benzyl)cyclopropyl)pyrimidin- 4(3H)-one | 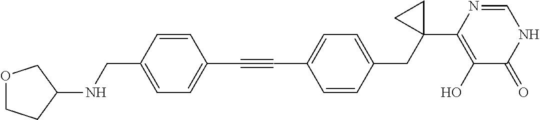 [SMILES: O=c1[nH]cnc(C2(Cc3ccc(C#Cc4ccc(CNC5CCOC5)cc4)cc3)CC2)c1O] | |
| 137 | 3-hydroxy-2-(3-hydroxy-2- (4-((4- (morpholinomethyl)phenyl) ethynyl)phenyl)propyl)-4H- pyran-4-one | 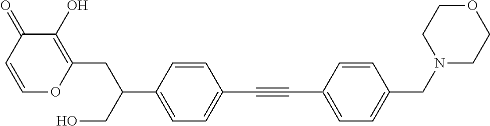 [SMILES: O=c1ccoc(CC(CO)c2ccc(C#Cc3ccc(CN4CCOCC4)cc3)cc2)c1O] | |
| 138 | 1-hydroxy-6-(3-hydroxy-2- (4-((4-(morpholinomethyl)phenyl) ethynyl)phenyl)propyl)pyridin- 2(1H)-one | 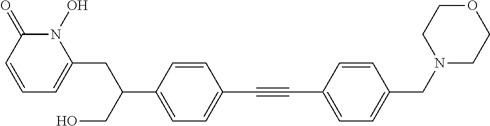 [SMILES: O=c1cccc(CC(CO)c2ccc(C#Cc3ccc(CN4CCOCC4)cc3)cc2)n1O] | |
| 139 | 2-(3-hydroxy-2-(4-((4- (morpholinomethyl)phenyl) ethynyl)phenyl)propyl)pyridine- 3,4-diol | 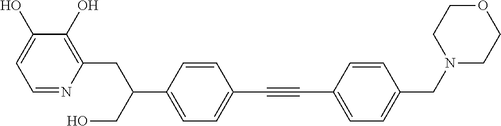 [SMILES: OCC(Cc1nccc(O)c1O)c1ccc(C#Cc2ccc(CN3CCOCC3)cc2)cc1] | |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

In some embodiments, the compounds described herein are labeled by other means, including, but not limited to, the use of chromophores or fluorescent moieties, bioluminescent labels, or chemiluminescent labels.

In one embodiment, the compounds disclosed herein contain one or more boron atom, silicon atom, or any combination thereof. In some embodiments, one or more carbon atoms in the compound disclosed herein are replaced with a boron atom, a silicon atom, or any combination thereof.

In some embodiments, one or more carbon atoms in the compound disclosed herein are replaced with a boron atom. In some embodiments, one carbon atom in the compound disclosed herein is replaced with a boron atom. In some embodiments, two carbon atoms in the compound disclosed herein are replaced with two boron atoms. In some embodiments, three carbon atoms in the compound disclosed herein are replaced with three boron atoms. In some embodiments, four carbon atoms in the compound disclosed herein are replaced with four boron atoms. In some embodiments, five carbon atoms in the compound disclosed herein are replaced with five boron atoms.

In some embodiments, one or more carbon atoms in the compound disclosed herein are replaced with a silicon atom. In some embodiments, one carbon atom in the compound disclosed herein is replaced with a silicon atom. In some embodiments, two carbon atoms in the compound disclosed herein are replaced with two silicon atoms. In some embodiments, three carbon atoms in the compound disclosed herein are replaced with three silicon atoms. In some embodiments, four carbon atoms in the compound disclosed herein are replaced with four silicon atoms. In some embodiments, five carbon atoms in the compound disclosed herein are replaced with five silicon atoms.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The disclosure provides for methods of treating diseases by administering such solvates. The disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. In some instances, the heterocyclic LpxC inhibitory compounds disclosed herein exist in tautomeric forms. The structures of said compounds are illustrated in the one tautomeric form for clarity. The alternative tautomeric forms are expressly included in this disclosure, such as, for example, the structures illustrated below.

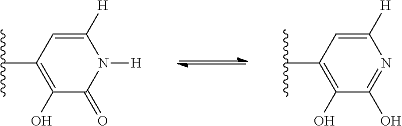 [SMILES: *c1cc[nH]c(=O)c1O ; *c1ccnc(O)c1O]

### Preparation of Compounds

The compounds used in the chemical reactions described herein are made according to organic synthesis techniques known to those skilled in this art, starting from commercially available chemicals and/or from compounds described in the chemical literature. “Commercially available chemicals” are obtained from standard commercial sources including Acres Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modem Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Alternatively, specific and analogous reactants can be identified through the indices of known chemicals and reactions prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the heterocyclic LpxC inhibitory compound described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the heterocyclic LpxC inhibitory compound as described herein is administered as a pure chemical. In other embodiments, the heterocyclic LpxC inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Provided herein is a pharmaceutical composition comprising at least one heterocyclic LpxC inhibitory compound, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

One embodiment provides a pharmaceutical composition comprising a compound disclosed herein, or a pharmaceutically acceptable salt thereof, and a pharmaceutically acceptable excipient.

In certain embodiments, the heterocyclic LpxC inhibitory compound as described herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See. e.g., Remington: The Science and Practice of Pharmacy (Gennaro, 21^{st} Ed. Mack Pub. Co., Easton, Pa. (2005)).

The dose of the composition comprising at least one heterocyclic LpxC inhibitory compound as described herein differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### LpxC, Lipid a and Gram-Negative Bacteria

Metalloproteins influence a vast diversity of biological systems, biological processes, and diseases. For example, UDP-{3-O—[(R)-3-hydroxymyristoyl]}-N-acetylglucosamine deacetylase (LpxC) is an essential enzyme involved in the first committed step in lipid A biosynthesis for gram-negative bacteria. Lipid A is an essential component of the outer membrane of gram-negative bacteria. LpxC is a zinc(II)-dependent metalloenzyme, with two histidines and an aspartic acid residue bound to the zinc(II) ion. Structures of LpxC show the zinc(II) ion is bound to two water molecules, both of which have been implicated in the mechanism of the enzyme. LpxC is highly conserved across strains of gram-negative bacteria, making LpxC an attractive target to treat gram-negative infections.

In recent years, there has been an increase in resistant and multi-drug resistant strains of bacteria. Thus, there is a need for new antibiotics, especially with new mechanisms of action. There remains a need for metalloprotein modulators of LpxC useful in the field of therapeutics, diagnostics, and research.

One embodiment provides a method of inhibiting UDP-{3-O—[(R)-3-hydroxymyristoyl]}-N-acetylglucosamine deacetylase enzyme comprising contacting the enzyme with a compound described herein.

One embodiment provides a method of modulating the activity of UDP-{3-O—[(R)-3-hydroxymyristoyl]}-N-acetylglucosamine deacetylase enzyme comprising contacting the enzyme with a compound described herein.

### Methods of Treatment

Disclosed herein are methods of treating disease wherein the inhibition of bacterial growth is indicated. Such disease includes gram-negative bacterial infection. In some embodiments, the method of treating a gram-negative bacterial infection in a patient in need thereof comprises administering to the patient a pharmaceutical composition comprising a compound disclosed herein, or a pharmaceutically acceptable salt thereof, and a pharmaceutically acceptable excipient. In some embodiments, the gram-negative bacterial infection is selected from pneumonia, sepsis, cystic fibrosis, intra-abdominal infection, skin infections and urinary tract infection. In some embodiments, the gram-negative bacterial infection is a urinary tract infection (UTI), a hospital acquired/ventilator-associated pneumonia (HAP/VAP), or an intra-abdominal infection (IAI). In some embodiments, the gram-negative bacterial infection is selected from chronic urinary tract infections, complicated urinary tract infections, cystitis, pyelonephritis, urethritis, recurrent urinary tract infections, bladder infections, urethral infections, or kidney infections. In some embodiments, the compounds described herein are used for the treatment of chronic urinary tract infections. In some embodiments, the compounds described herein are used for the treatment of complicated urinary tract infections. In other embodiments, the compounds described herein are used for the treatment of complicated intra-abdominal infection. In some embodiments, the compounds described herein are used for the treatment of chronic intra-abdominal infection. In other embodiments, the compounds described herein are used for the treatment of hospital acquired pneumonia (HAP) or ventilator associated pneumonia (VAP). In some embodiments the administration is to treat an existing infection. In some embodiments the administration is provided as prophylaxis.

In some embodiments the heterocyclic LpxC inhibitory compound as described herein is used for treating conditions caused by the bacterial production of endotoxin and, in particular, by gram-negative bacteria and bacteria that use LpxC in the biosynthesis of lipopolysaccharide (LPS) or endotoxin. In some embodiments, the method of treating a condition caused by endotoxin or LPS in a patient in need thereof comprises administering to the patient a pharmaceutical composition comprising a compound described herein, or a pharmaceutically acceptable salt thereof, and a pharmaceutically acceptable excipient. In another embodiment, the heterocyclic LpxC inhibitory compounds as described herein are useful in the treatment of conditions that are caused or exacerbated by the bacterial production of lipid A and LPS or endotoxin, such as sepsis, septic shock, systemic inflammation, localized inflammation, chronic obstructive pulmonary disease (COPD) and acute exacerbations of chronic bronchitis (AECB). In some embodiments, the method of treating a condition caused by endotoxin or LPS in a patient in need thereof comprises administering to the patient a pharmaceutical composition comprising a compound disclosed herein, or a pharmaceutically acceptable salt thereof, and a pharmaceutically acceptable excipient, wherein the condition caused by endotoxin or LPS is selected from sepsis, septic shock, systemic inflammation, localized inflammation, chronic obstructive pulmonary disease (COPD) and acute exacerbations of chronic bronchitis (AECB).

In other embodiments, the compounds of the disclosure can be used for the treatment of a serious or chronic respiratory tract infection or complicated urinary tract infections including serious lung and nosocomial infections such as those caused by Enterobacter aerogenes, Enterobacter cloacae, Escherichia coli, Klebsiella pneumoniae, Klebsiella oxytoca, Proteus mirabilis, Serratia marcescens, Stenotrophomonas maltophilia. Pseudomonas aeruginosa, Burkholderia cepacia, Acinetobacter baumannii, Alcaligenes xylosoxidans, Flavobacterium meningosepticum, Providencia sluarlii and Citrobacter freundi, Haemophilus influenzae, Legionella species, Moraxella catarrhalis, Enterobacter species, Acinetobacter species, Klebsiella species, Burkholderia species and Proteus species, and infections caused by other bacterial species such as Neisseria species, Shigella species, Salmonella species, Helicobacler pylori, Vibrionaceae and Bordetella species as well as the infections caused by a Brucella species, Francisella tularensis and/or Yersinia pestis.

In other embodiments, the compounds of the disclosure are not active against gram-positive bacteria. In some embodiments, the compounds of the disclosure are not active against Staphylococcus aureus.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants, J are reported in Hertz. For proton spectra the solvent peak was used as the reference peak.

### Example 1

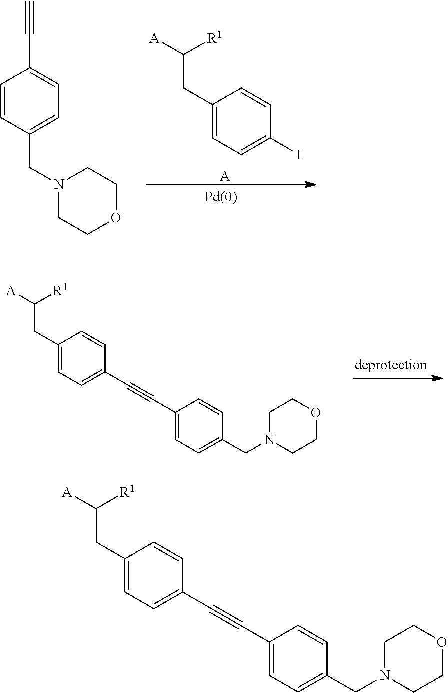

### Synthesis of 4-(4-ethynylbenzyl) morpholine

To a stirred solution of 4-ethynylbenzaldehyde (5.0 g, 0.0384 mol) in MeOH (20 mL), morpholine (5.0 g, 5.0 mL, 0.058 mol) was added along with 0.1 mL of AcOH. The reaction was stirred for 1 h until a homogenous solution was visible. To the reaction mixture NaCNBH₃ (2.90 g, 1.2 eq, 0.046 mol) was added and stirred for a period of 2 h. After completion, the reaction mixture was concentrated to dryness under vacuum. The reaction mixture was partitioned between water (50 mL) and EtOAc (100 mL). The water layer was slightly acidified with dil. HCl for a better separation. The aqueous layer was further extracted with EtOAc (100 mL). The organic layers were combined washed over satd. NaCl (50 mL), dried over Na₂SO₄ and concentrated to dryness to get a crude product. The crude product was further purified on a column chromatography on silica gel to get the desired product (6.7 g, 87%). LCMS=Calculated for C₁₃H₁₅NO is 201.27, observed=202.27.

### Palladium-Catalyzed Cross-Coupling Reaction

To a solution of aryliodide A is added 4-(4-ethynylbenzyl) morpholine and Et₃N. The reaction mixture is sparged with nitrogen for 10 min, PdCl₂(PPh₃)₂ and CuI is added and the reaction mixture is stirred at 100° C. for 1 h. After completion of the reaction, solvent is removed under reduced pressure and the reaction mixture is dissolved in water and extracted with EtOAc (2×50 mL). The combined organic layers are washed with satd. NaCl (50 mL), dried over Na₂SO₄. filtered and concentrated and the crude product is purified by column chromatography on silica gel (230-400 mesh, 15% MeOH in DCM) to obtain the desired product.

The product of the cross-coupling reaction described above is deprotected. The crude product is purified by reverse phase HPLC to afford the desired product.

### Example 2

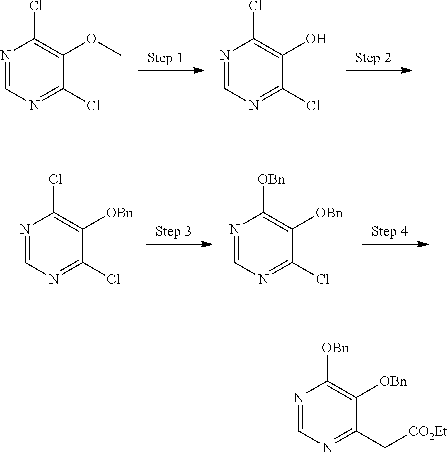

Step 1—To a stirred solution of 4,6-dichloro-5-methoxypyrimidine (50 g, 0.27 mol) in dichloroethane (800 mL) at 0° C., anhydrous aluminum chloride (50.48 g, 0.411 mol) was added in a single portion. The reaction mixture was stirred vigorously at 50° C. for 6 h. After completion, the mixture was cooled to 0° C. and aqueous HCl solution (1 M, 400 mL) was added slowly followed by addition of MeOH (100 mL). The mixture was stirred vigorously at room temperature for 10 min, diluted with water and extracted with EtOAc. The combined organic layers were washed with satd. NaCl, dried over Na₂SO₄, filtered and concentrated to afford 4, 6-dichloropyrimidin-5-ol (41 g, 91.11%). UPLC=Calculated for C₄H₂Cl₂N₂O is 164.97, Observed=165.9.

Step 2—To a stirred solution of 4, 6-dichloropyrimidin-5-ol (6.5 g, 35.50 mmol) in DMF (120 mL), benzyl bromide (8.42 mL, 70.90 mmol) was added followed by the addition of potassium carbonate (14.70 g, 106.36 mmol). The reaction mixture was stirred at 60° C. for 1 h. The mixture was concentrated and the residue was partitioned between EtOAc (150 mL) and ice cold water (75 mL). The aqueous layer was further extracted with EtOAc (2 * 100 mL). The organic layers were combined and washed with satd. NaCl, dried over Na₂SO₄, filtered and concentrated. The residue was purified by flash chromatography on silica gel (230-400 mesh, 10% EtOAc in pet. ether) to afford 5-(benzyloxy)-4, 6-dichloropyrimidine (9.5 g, 94%). LCMS=Calculated for C₁₁H₈Cl₂N₂O is 255.10, Observed=256.1.

Step 3—To a solution of benzyl alcohol (3.8 g, 0.0352 mol) in THF (100 mL), NaH (60% in mineral oil, 1.4 g, 0.0352 mol) was added at 0° C. and stirred for 30 min. To this a solution of 5-(benzyloxy)-4, 6-dichloropyrimidine (9 g, 0.0352 mol) in THF (20 mL) was added at 0° C. and stirred for 30 min. After completion of the reaction, the reaction mixture was diluted with water and extracted with EtOAc (2×100 mL). The organic layers were combined and washed with satd. NaCl, dried over Na₂SO₄, filtered and concentrated under reduced pressure to afford 4, 5-bis (benzyloxy)-6-chloropyrimidine (11 g, 95.6%). UPLC=Calculated for C₁₈H₁₅ClN₂O₂ is 326.78, Observed=327.6.

Step 4—To a solution of 4,5-bis (benzyloxy)-6-chloropyrimidine (10 g, 0.0306 mol) in dry toluene (20 mL), diethyl malonate (29.4 g, 0.183 mol) and t-BuOK (10.29 g, 0.0918 mol) were added and refluxed at 120° C. for 3 h. After completion of the reaction, the reaction mixture was diluted with water and extracted with EtOAc (2×100 mL). The combined organic layers were washed with satd. NaCl, dried over anhydrous Na₂SO₄, filtered and concentrated under reduced pressure. The residue was purified by flash chromatography on silica gel (230-400 mesh, 15% EtOAc in pet. ether) to afford ethyl 2-(5, 6-bis (benzyloxy) pyrimidin-4-yl) acetate (3 g, 26%). UPLC=Calculated for C₂₂H₂₂N₂O₄ is 378.43, Observed=379.4.

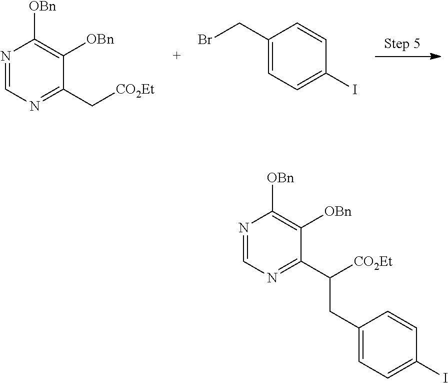 [SMILES: CCOC(=O)Cc1ncnc(OCc2ccccc2)c1OCc1ccccc1 ; BrCc1ccc(I)cc1 ; CCOC(=O)C(Cc1ccc(I)cc1)c1ncnc(OCc2ccccc2)c1OCc1ccccc1]

Step 5—To a solution of ethyl 2-(5, 6-bis (benzyloxy) pyrimidin-4-yl) acetate (3 g, 0.0079 mol) in DMF (30 mL) NaH (60%, 0.317 g, 0.0079 mol) was added at 0° C. and stirred for 30 min. Then 4-Iodo benzyl bromide (2.35 g, and 0.0079 mol) was added and stirred at 0° C. for 2 h. After completion of the reaction, the reaction mixture was quenched with sat. NH₄Cl and diluted with water and extracted with EtOAc (2×100 mL). The combined organic layers were washed with satd. NaCl, dried over Na₂SO₄, filtered and concentrated under vacuum. The crude product was purified by column chromatography on silica gel (230-400 mesh, 10% EtOAc in pet. ether) to afford ethyl 2-(5, 6-bis (benzyloxy) pyrimidin-4-yl)-3-(4-iodophenyl) propanoate (4.4 g, 93.6%). LCMS=Calculated for C₂₉H₂₇IN₂O₄ is 594.45, Observed=595.33.

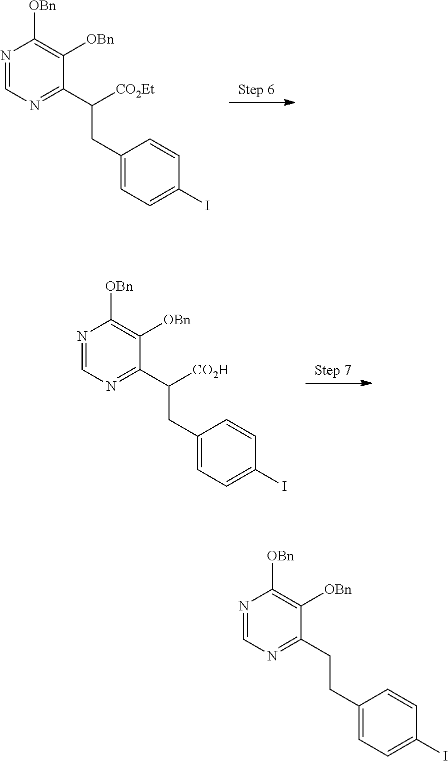 [SMILES: CCOC(=O)C(Cc1ccc(I)cc1)c1ncnc(OCc2ccccc2)c1OCc1ccccc1 ; O=C(O)C(Cc1ccc(I)cc1)c1ncnc(OCc2ccccc2)c1OCc1ccccc1 ; Ic1ccc(CCc2ncnc(OCc3ccccc3)c2OCc2ccccc2)cc1]

Step 6—To a solution of 2-(5, 6-bis (benzyloxy) pyrimidin-4-yl)-3-(4-iodophenyl) propanoate (1.4 g, 0.0029 mol) in EtOH:THF (1:1) (10 mL), water (10 mL) was added followed by NaOH (0.59 g, 0.0148 mol) and the reaction mixture was stirred for 4 h. After completion of the reaction, the reaction mixture was diluted with water and extracted with diethyl ether (100 mL). The organic layer was separated and the aqueous layer was acidified with 1.5N HCl to pH 6-7 and extracted with EtOAc (2 * 50 mL). The combined organic layers were washed with satd. NaCl, dried over Na₂SO₄, filtered and concentrated under reduced pressure to afford 2-(5,6-bis(benzyloxy)pyrimidin-4-yl)-3-(4-iodophenyl)propanoic acid (1.2 g, 91.6%). UPLC=Calculated for C₂₁H₁₉IN₂O₄ is 566.40, Observed=567.20

### Synthesis of 4,5-bis(benzyloxy)-6-(4-iodophenethyl)pyrimidine

Step 7—The compound 2-(5,6-bis(benzyloxy)pyrimidin-4-yl)-3-(4-iodophenyl)propanoic acid (1 g, 0.0017 mol) obtained from previous step is heated at 67° C. in a water bath under vacuum for a period of 1 h. After completion, the reaction mixture was diluted with DCM and purified on a column chromatography using silica gel using EtOAc and hexanes to get the desired compound 4,5-bis(benzyloxy)-6-(4-iodophenethyl)pyrimidine (0.770 g, 87%). LCMS=Calculated for C₂₆H₂₃IN₂O is 522.39, observed=523.39.

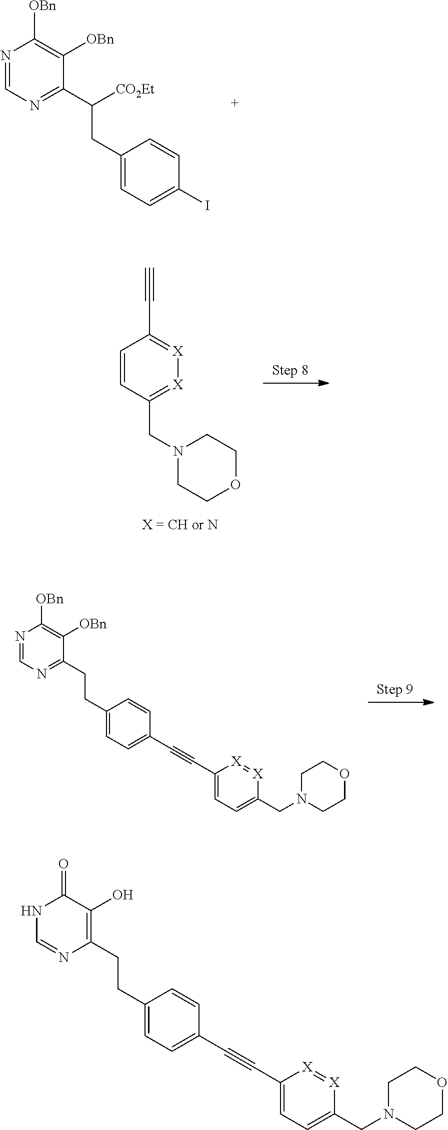

Step 8—To a solution of 4,5-bis(benzyloxy)-6-(4-iodophenethyl)pyrimidine in toluene:dioxane (1:1)(10 mL) was added a morpholino substituted heteroaryl acetylene, and Et₃N. The reaction mixture was bubbled with nitrogen for 10 min, PdCl₂(PPh₃)₂ was added followed by CuI and the reaction mixture was stirred at 100° C. for 1 h. After completion of the reaction, solvent was removed under reduced pressure and the reaction mixture was dissolved in water and extracted with EtOAc (2×50 mL). The combined organic layers were washed with satd. NaCl (50 mL), dried over Na₂SO₄, filtered and concentrated and the crude product was purified by column chromatography on silica gel (230-400 mesh, 15% MeOH in. DCM) to get the desired product.

Step 9—To a solution of the product of step 8 in DCM (5 mL), BCl₃ (1M in DCM, 10 mL) was added and stirred at 25° C. for 2 h. Then the reaction mixture was quenched with methanol and neutralized to pH=7-8 and concentrated under reduced pressure. The crude product was purified by reverse phase HPLC to afford the desired product as a formate salt.

### Example 3

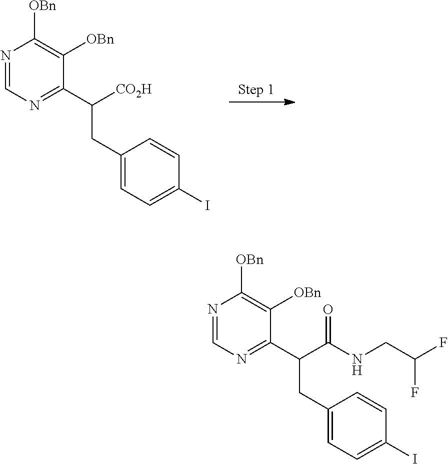 [SMILES: O=C(O)C(Cc1ccc(I)cc1)c1ncnc(OCc2ccccc2)c1OCc1ccccc1 ; O=C(NCC(F)F)C(Cc1ccc(I)cc1)c1ncnc(OCc2ccccc2)c1OCc1ccccc1]

Step 1—To a stirred solution of 2-(5,6-bis(benzyloxy)pyrimidin-4-yl)-3-(4-iodophenyl)propanoic acid in DCM, amine was added followed by EDC.HCl and HOBT and the reaction mixture was stirred for a period of 4 h. After completion, the reaction mixture was diluted with DCM and washed with satd. NaHCO₃ solution followed by satd. NaCl and dried further over Na₂SO₄ and concentrated under vacuum to get a crude product. The crude product was purified on a column chromatography on a silica gel using EtOAc (90%) in hexanes to get the desired product.

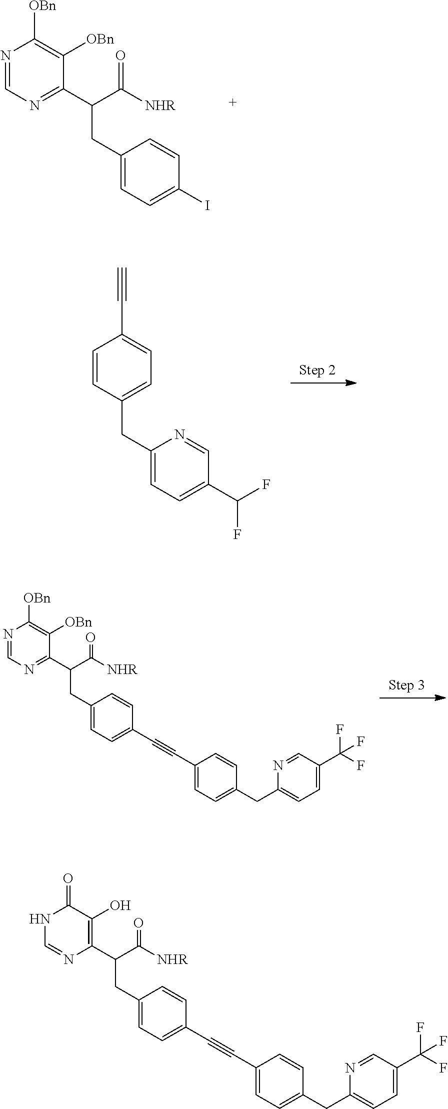

Step 2—To a solution of the product of step 1 in toluene:dioxane (1:1) was added 2-(4-ethynylbenzyl)-5-(trifluoromethyl)pyridine, Et₃N. The reaction mixture was bubbled with nitrogen for 10 min, PdCl₂(PPh₃)₂ was added followed by CuI and the reaction mixture was stirred at 100° C. for 1 h. After completion of the reaction, solvent was removed under reduced pressure and the reaction mixture was dissolved in water and extracted with EtOAc (2×50 mL). The combined organic layers were washed with satd. NaCl (50 mL), dried over Na₂SO₄, filtered and concentrated and the crude product was purified by column chromatography on silica gel (230-400 mesh, 15% MeOH in. DCM) to get the desired product.

Step 3—To a solution of the product of step 2 in DCM, BCl₃ (1M in DCM) was added and stirred at 25° C. for 2 h. Then the reaction mixture was quenched with methanol and neutralized to pH=7-8 and concentrated under reduced pressure. The crude product was purified by reverse phase HPLC to afford the desired product as a formate salt.

### Example 4

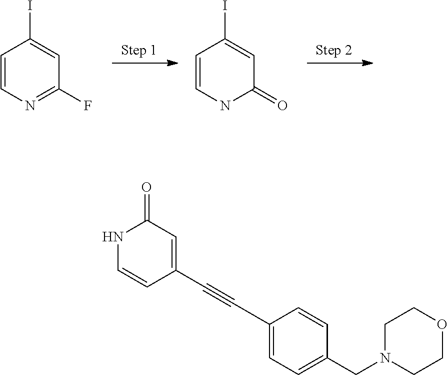 [SMILES: Fc1cc(I)ccn1 ; O=c1cc(I)cc[nH]1 ; O=c1cc(C#Cc2ccc(CN3CCOCC3)cc2)cc[nH]1]

Step 1—A solution of 2-fluoro-4-iodopyridine (2 g, 8.96 mmol) in acetic acid (20 mL) and water (5 mL), was heated to 100° C. for 5 h. After completion of the reaction, acetic acid was removed under reduced pressure. The crude product was triturated with diethylether, solid was dried to get pure, 4-iodopyridin-2(1H)-one as off white solid. Yield: 1.2 g, 60.6%. LCMS: Calculated for C₅H₄INO 221.00, Observed=221.9

Step 2—To a solution of 4-iodopyridin-2(1H)-one (0.36 g, 1.62 mmol) and 4-(4-ethynylbenzyl) morpholine (0.65 g, 3.25 mmol) in dry DMF (8 mL) was added TEA (0.9 mL, 6.48 mmol). The reaction mixture was degassed by purging with N₂ for 15 minutes. Then PdCl₂(PPh₃)₂ (22 mg, 0.03 mmol) followed by copper (I) iodide (18 mg, 0.097 mmol) were added and the reaction mixture was stirred at 25° C. for 1 h. After completion of the reaction, the reaction mixture was diluted with water (10 mL) and the crude product was extracted with EtOAc (3×10 mL). The combined organic layer was washed with water (2×10 mL) and finally with brine solution (10 mL). The organic layer was dried over anhydrous Na₂SO₄, filtered and concentrated. Crude product was purified by column chromatography over silica gel (230-400 Mesh) (eluent: 5-7% MeOH in DCM) to obtain 4-((4-(morpholinomethyl)phenyl) ethynyl)pyridin-2(1H)-one as a brown solid. Yield: 0.26 g, 55.3%. LCMS: Calculated for C₁₈H₁₈N₂O₂ is 294.35, Observed=295.2.

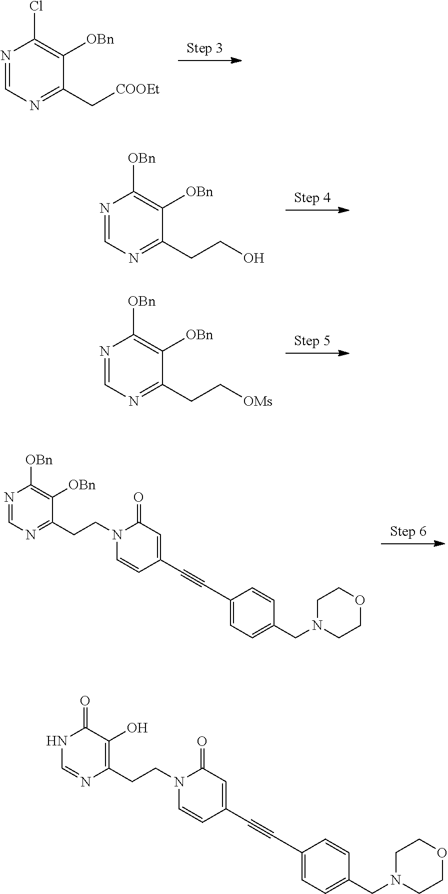 [SMILES: CCOC(=O)Cc1ncnc(Cl)c1OCc1ccccc1 ; OCCc1ncnc(OCc2ccccc2)c1OCc1ccccc1 ; CS(=O)(=O)OCCc1ncnc(OCc2ccccc2)c1OCc1ccccc1 ; O=c1cc(C#Cc2ccc(CN3CCOCC3)cc2)ccn1CCc1ncnc(OCc2ccccc2)c1OCc1ccccc1 ; O=c1[nH]cnc(CCn2ccc(C#Cc3ccc(CN4CCOCC4)cc3)cc2=O)c1O]

Step 3—A solution of ethyl 2-(5-(benzyloxy)-6-chloropyrimidin-4-yl)acetate (1.5 g, 3.96 mmol) in THF (20 mL) was cooled to −30° C. and DIBAL-H (11.8 mL, 11.9 mmol) was added slowly and stirred the reaction mixture at 25° C. for 2 h. After completion of the reaction, reaction mixture was quenched with sat. NH₄Cl solution. The reaction mixture was filtered on celite bed and bed was washed with ethyl acetate. The layers were separated organic layer was dried over anhydrous Na2SO4, filtered and concentrated under reduced pressure to get pure 2-(5,6-bis(benzyloxy)pyrimidin-4-yl)ethan-1-ol. Yield: 1.1 g, 82.7%. LCMS: Calculated for C₂₀H₂₀N₂O₃ 336.39, Observed=337.2.

Step 4—To a 0° C. cooled solution of 2-(5,6-bis(benzyloxy)pyrimidin-4-yl)ethan-1-ol (0.1 g, 0.29 mmol) in DCM (3 mL), triethylamine (0.06 mL, 0.43 mmol) was added followed by methane sulfonyl chloride (0.04 g, 0.35 mmol). After completion of the reaction, the reaction mixture was washed with water and brine solution. The organic layer was dried over anhydrous Na₂SO₄, filtered and concentrated under reduced pressure to get pure 2-(5,6-bis(benzyloxy)pyrimidin-4-yl)ethyl methanesulfonate. Yield: 0.105 g, 82.9% LC_MS: Calculated for C₂₁H₂₂N₂O₅S is 414.48, Observed=415.2.

Step 5—To a stirred solution of 4-((4-(morpholinomethyl)phenyl) ethynyl)pyridin-2(1H)-one (0.12 g, 0.88 mmol) and 2-(5,6-bis(benzyloxy)pyrimidin-4-yl)ethyl methanesulfonate (0.25 g, 0.88 mmol) in DMSO (5 mL) was added Cs₂CO₃ (0.43 g, 1.3 mmol). The reaction mixture was heated at 80° C. for 2 hours. After completion of the reaction, the reaction mixture was diluted with water (30 mL) and the crude product was extracted with EtOAc (3×10 mL). The combined organic layer was washed with water (2×20 mL) and brine solution (20 mL). The organic layer was dried over anhydrous Na₂SO₄, filtered and concentrated. The crude product was purified by automated flash column chromatography over silica gel (eluent: 90-100% EtOAc in Pet ether) to obtain l-(2-(5,6-bis(benzyloxy)pyrimidin-4-yl)ethyl)-4-((4-(morpholinomethyl)phenyl) ethynyl)pyridin-2(1H)-one as a white solid. Yield: 0.048 g, 10%. LCMS: Calculated for C₃₈H₃₆N₄O₄ 612.73, Observed=613.3.

Step 6—To a solution of 1-(2-(5,6-bis(benzyloxy)pyrimidin-4-yl)ethyl)-4-((4-(morpholinomethyl)phenyl) ethynyl)pyridin-2(1H)-one (0.04 g, 0.065 mmol) in DCM, BCl₃ (1M in DCM, 2 mL) was added and stirred the reaction mixture at 25° C. for 2 h. After completion of the reaction, the reaction mixture was carefully quenched with MeOH and stirred. After 10 min the reaction mixture was concentrated under reduced pressure. The crude product was purified by prep HPLC to get pure 5-hydroxy-6-(2-(4-((4-(morpholinomethyl)phenyl)ethynyl)-2-oxopyridin-1(2H)-yl)ethyl)pyrimidin-4(3H)-one as an off white solid. Yield: 14 mg, 50%. LCMS=Calculated for C₂₄H₂₄N₄O₄ 432.48, Observed=433.2.

### Example 5

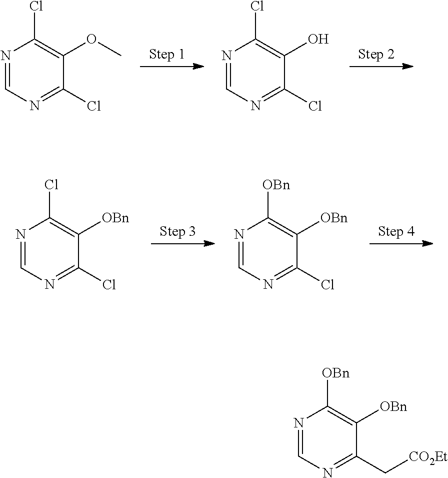

Step 1—To a stirred solution of 4,6-dichloro-5-methoxypyrimidine (50 g, 0.27 mol) in dichloroethane (800 mL) at 0° C., anhydrous aluminum chloride (50.48 g, 0.411 mol) was added in a single portion. The reaction mixture was stirred vigorously at 50° C. for 6 h. After completion, the mixture was cooled to 0° C. and aqueous HCl solution (1 M, 400 mL) was added slowly followed by addition of MeOH (100 mL). The mixture was stirred vigorously at room temperature for 10 min, diluted with water and extracted with EtOAc. The combined organic layers were washed with satd. NaCl, dried over Na₂S04, filtered and concentrated to afford 4, 6-dichloropyrimidin-5-ol (41 g, 91.11%). UPLC=Calculated for C₄H₂Cl₂N₂O is 164.97, Observed=165.9.

Step 2—To a stirred solution of 4, 6-dichloropyrimidin-5-ol (6.5 g, 35.50 mmol) in DMF (120 mL), benzyl bromide (8.42 mL, 70.90 mmol) was added followed by the addition of potassium carbonate (14.70 g, 106.36 mmol). The reaction mixture was stirred at 60° C. for 1 h. The mixture was concentrated and the residue was partitioned between EtOAc (150 mL) and ice cold water (75 mL). The aqueous layer was further extracted with EtOAc (2 * 100 mL). The organic layers were combined and washed with satd. NaCl, dried over Na₂S04, filtered and concentrated. The residue was purified by flash chromatography on silica gel (230-400 mesh, 10% EtOAc in pet. ether) to afford 5-(benzyloxy)-4, 6-dichloropyrimidine (9.5 g, 94%). LCMS=Calculated for C₁₁H₈Cl₂N₂O is 255.10, Observed=256.1.

Step 3—To a solution of benzyl alcohol (3.8 g, 0.0352 mol) in THE (100 mL), NaH (60% in mineral oil, 1.4 g, 0.0352 mol) was added at 0° C. and stirred for 30 min. To this a solution of 5-(benzyloxy)-4, 6-dichloropyrimidine (9 g, 0.0352 mol) in THF (20 mL) was added at 0° C. and stirred for 30 min. After completion of the reaction, the reaction mixture was diluted with water and extracted with EtOAc (2×100 mL). The organic layers were combined and washed with satd. NaCl, dried over Na₂SO₄, filtered and concentrated under reduced pressure to afford 4, 5-bis (benzyloxy)-6-chloropyrimidine (11 g, 95.6%). UPLC=Calculated for C₁₈H₁₅ClN₂O₂ is 326.78, Observed=327.6.

Step 4—To a solution of 4,5-bis (benzyloxy)-6-chloropyrimidine (10 g, 0.0306 mol) in dry toluene (20 mL), diethyl malonate (29.4 g, 0.183 mol) and t-BuOK (10.29 g, 0.0918 mol) were added and refluxed at 120° C. for 3 h. After completion of the reaction, the reaction mixture was diluted with water and extracted with EtOAc (2×100 mL). The combined organic layers were washed with satd. NaCl, dried over anhydrous Na₂SO₄, filtered and concentrated under reduced pressure. The residue was purified by flash chromatography on silica gel (230-400 mesh, 15% EtOAc in pet. ether) to afford ethyl 2-(5, 6-bis (benzyloxy) pyrimidin-4-yl) acetate (3 g, 26%). UPLC=Calculated for C₂₂H₂₂N₂O₄ is 378.43, Observed=379.4.

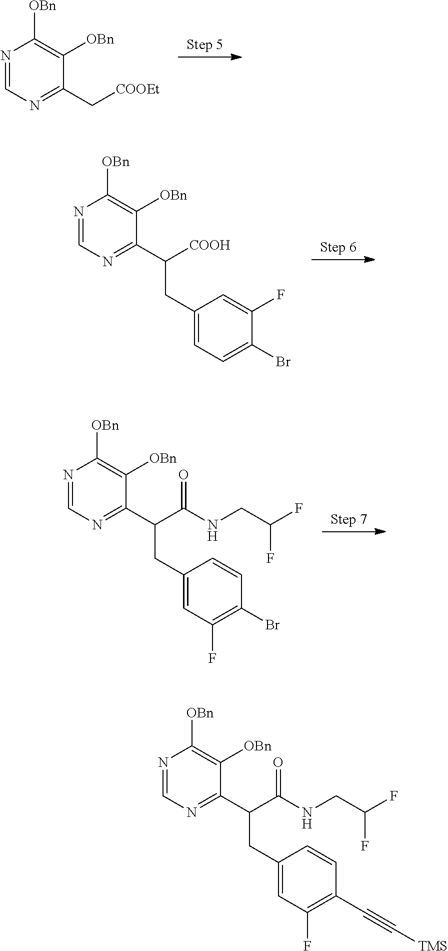 [SMILES: CCOC(=O)Cc1ncnc(OCc2ccccc2)c1OCc1ccccc1 ; O=C(O)C(Cc1ccc(Br)c(F)c1)c1ncnc(OCc2ccccc2)c1OCc1ccccc1 ; O=C(NCC(F)F)C(Cc1ccc(Br)c(F)c1)c1ncnc(OCc2ccccc2)c1OCc1ccccc1 ; C[Si](C)(C)C#Cc1ccc(CC(C(=O)NCC(F)F)c2ncnc(OCc3ccccc3)c2OCc2ccccc2)cc1F]

Step 5—To a solution of ethyl 2-(5, 6-bis (benzyloxy) pyrimidin-4-yl) acetate (5 g, 0.0132 mol) in DMF (40 mL), NaH (60%, 0.63 g, 0.037 mol) was added at 0° C. and stirred for 10 min. To this cooled solution 1-bromo-4-(bromomethyl)-2-fluorobenzene (3.6 g, and 0.0132 mol) was added and slowly allowed stirred at 25° C. for 1 h. After completion of the reaction, the reaction mixture was quenched with sat. NH₄Cl and diluted with water and extracted with EtOAc (2* 100 mL). The combined organic layers were washed with brine solution, organic layer was dried over Na₂SO₄, filtered and concentrated under reduced pressure. The crude product was purified by column chromatography on silica gel (230-400 mesh, 10% EtOAc in pet. ether) to afford 2-(5,6-bis(benzyloxy)pyrimidin-4-yl)-3-(4-bromo-3-fluorophenyl)propanoic acid. Yield: 5.1, 68.3% LC_MS=Calculated for C29H26BrFN2O4 is 565.44, Observed=565.2

Step 6—To a slurry of 2-(5,6-bis(benzyloxy)pyrimidin-4-yl)-3-(4-bromo-3-fluorophenyl)propanoic acid (2 g, 0.00353 mol) in a mixture of ethanol:water (20 ml:10 ml), NaOH (0.7 g, 0.0176 mol) was added and stirred for 3 h at 25° C. After completion of the reaction, the reaction mixture was concentrated and the resultant crude was diluted with water and acidified with Cone. HCl (adjusted pH=2). The reaction mixture was extracted with EtOAc (10 mL*2). The combined organic layers were dried over Na₂SO₄, filtered, concentrated under reduced pressure to get 1.7 g of crude acid. The crude product (1.7 g, 0.0031 mol) was dissolved in DMF (20 mL), to this 2,2-difluoroethan-1-amine (0.51 g, 0.0063 mol), HATU (1.7 g, 0.00465 mol) and DIPEA (5.1 mL, 0.0062 mol) were added at 25° C. and stirred for 4 h. After completion of the reaction, the reaction mixture was diluted with water and extracted with EtOAc (100 mL*2). The combined organic layers were dried over Na₂SO₄, filtered, concentrated under reduced pressure. The crude product was purified by column chromatography on silica gel (230-400 mesh, 10% EtOAc in pet. ether) to afford 2-(5,6-bis(benzyloxy)pyrimidin-4-yl)-3-(4-bromo-3-fluorophenyl)-N-(2,2-difluoroethyl)propanamide. Yield: (1.32 g, 71%). LC_MS=Calculated for C₂₉H₂₅BrF₃N₃O₃ is 600.44, Observed=603.2

Step 7—To a solution of 2-(5,6-bis(benzyloxy)pyrimidin-4-yl)-3-(4-bromo-3-fluorophenyl)-N-(2,2-difluoroethyl)propanamide (0.75 g, 0.0012 mol) in DMF (10 mF), Et₃N (1.65 mF, 0.0072 mol) and PPh₃ (0.032 g, 0.00012 mol) was added. To the degassed reaction mixture, PdCl₂(PPh₃)₂ (0.017 g, 0.000024 mol) and CuI (0.0148 g, 0.000077 mol) was added followed by TMS acetylene (1.8 g, 0.0186 mol) and the reaction mixture was heated at 120° C. for 16 h in a sealed tube. After completion of the reaction, solvent was removed under reduced pressure and the reaction mixture was dissolved in water and extracted with EtOAc (2×50 mF). The combined organic layers were washed with brine, dried over Na₂SO₄, filtered and concentrated and the crude product was purified by column chromatography on silica gel (230-400 mesh, 15% MeOH in DCM) to get 2-(5,6-bis(benzyloxy)pyrimidin-4-yl)-N-(2,2-difluoroethyl)-3-(3-fluoro-4-((trimethylsilyl)ethynyl)phenyl)propanamide. Yield: 0.25 g, mixture of SM and product. LCMS=Calculated for C₃₄H₃₄F₃N₃O₃Si is 617.74, Observed=618.2.

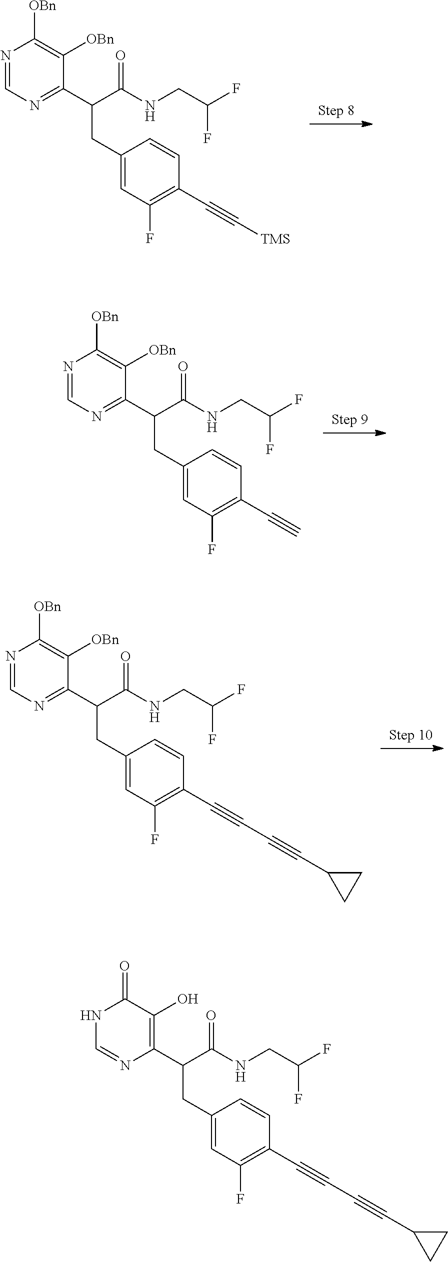 [SMILES: C[Si](C)(C)C#Cc1ccc(CC(C(=O)NCC(F)F)c2ncnc(OCc3ccccc3)c2OCc2ccccc2)cc1F ; C#Cc1ccc(CC(C(=O)NCC(F)F)c2ncnc(OCc3ccccc3)c2OCc2ccccc2)cc1F ; O=C(NCC(F)F)C(Cc1ccc(C#CC#CC2CC2)c(F)c1)c1ncnc(OCc2ccccc2)c1OCc1ccccc1 ; O=C(NCC(F)F)C(Cc1ccc(C#CC#CC2CC2)c(F)c1)c1nc[nH]c(=O)c1O]

Step 8—TBAF solution (1M in THF, 3 ml) was added to a solution 2-(5,6-bis(benzyloxy)pyrimidin-4-yl)-N-(2,2-difluoroethyl)-3-(3-fluoro-4-((trimethylsilyl)ethynyl)phenyl)propanamide (0.25 g, 0.0004 mol) in THF (5 mF) and stirred the reaction mixture at 25° C. for 15 min. After completion of the reaction, the reaction mixture was diluted with water and extracted with EtOAc (2×25 mF). The combined organic layers were washed with brine solution, dried over Na₂SO₄, filtered and concentrated to get crude, 2-(5,6-bis(benzyloxy)pyrimidin-4-yl)-N-(2,2-difluoroethyl)-3-(4-ethynyl-3-fluorophenyl)propanamide and the crude product was directly taken further step without any purification. Yield: (0.18 g, Crude) UPFC=Calculated for C₃₁H₂₆F₃N₃O₃ is 545.56, Observed=546.2

Step 9—The crude acetylene (0.12 g, 0.00029 mol) product and the cyclopropyl acetyelene (0.095 g, 0.00145 mol) were dissolved in mixture of MeOH (3 mF) and pyridine (3 mF). To this reaction mixture Cu(OAc)₂ (0.123 g, 0.00065 mol) was added and stirred the reaction mixture at 25° C. for 12 h. After completion of the reaction, the solvent was removed under reduced pressure and the reaction mixture was dissolved in water and extracted with EtOAc (2×50 mF). The combined organic layers were washed with brine, dried over Na₂SO₄, filtered and concentrated and the crude product was pass through the column chromatography on silica gel (60-120 mesh, 15% MeOH in DCM) to get 2-(5,6-bis(benzyloxy)pyrimidin-4-yl)-3-(4-(cyclopropylbuta-1,3-diyn-1-yl)-3-fluorophenyl)-N-(2,2-difluoroethyl)propanamide. Yield: 0.2 g, product and unreacted step 7 SM LCMS=Calculated for C₃₆H₃₀F₃N₃O₃ is 609.65, Observed=610.2

Step 10—To a solution of 2-(5,6-bis(benzyloxy)pyrimidin-4-yl)-3-(4-(cyclopropylbuta-1,3-diyn-1-yl)-3-fluorophenyl)-N-(2,2-difluoroethyl)propanamide (0.2 g, 0.0003 mol) in DCM, BCl₃ (1M in DCM) was added and stirred the reaction mixture at 25° C. for 2 h. After completion of the reaction, the reaction mixture was carefully quenched with MeOH and stirred. After 10 min. the reaction mixture was concentrated under reduced pressure. The crude product was purified by reverse phase prep HPLC (0.1% HCOOH in CH3CN) to get pure 3-(4-(cyclopropylbuta-1,3-diyn-1-yl)-3-fluorophenyl)-N-(2,2-difluoroethyl)-2-(5-hydroxy-6-oxo-1,6-dihydropyrimidin-4-yl)propanamide as off white solid. Yield: 5.2 mg, 4.7% LC_MS Calc. for C₂₂H₁₈F3N303 429.40; Obs. 430.0 [M⁻+H],

The compounds described herein were made according to organic synthesis analogous to those in Examples 1-5, as well as other techniques known to those skilled in this art, starting from commercially available chemicals and/or from compounds described in the chemical literature. The LC-MS data for each compound is shown in Table 1.

### II. Biological Evaluation

### Example 1: In Vitro Assays to Determine Bacterial Susceptibility

Minimal inhibitory concentrations (MIC) were determined by the broth microdilution method in accordance with the Clinical and Laboratory Standards Institute (CLSI) guidelines. In brief, organism suspensions were adjusted to a 0.5 McFarland standard to yield a final inoculum between 3×10⁵ and 7×10⁵ colony-forming units (CFU)/mL. Drug dilutions and inocula were made in sterile, cation adjusted Mueller-Hinton Broth (Beckton Dickinson). An inoculum volume of 100 μL was added to wells containing 100 μL of broth with 2-fold serial dilutions of drug. All inoculated microdilution trays were incubated in ambient air at 35° C. for 18-24 h. Following incubation, the lowest concentration of the drug that prevented visible growth (OD600 nm<0.05) was recorded as the MIC. Performance of the assay was monitored by the use of laboratory quality-control strains and levofloxacin, a compound with a defined MIC spectrum, in accordance with CLSI guidelines.

Exemplary in vitro assay data against select bacteria for compounds disclosed herein is provided in Table 2.

TABLE 2

| Compound No. | E. coli MIC | K. pneumoniae MIC |
|---|---|---|
| 1 | D | n.d. |
| 2 | C | n.d. |
| 3 | D | n.d. |
| 4 | D | n.d. |
| 5 | D | n.d. |
| 6 | D | n.d. |
| 7 | D | n.d. |
| 8 | D | D |
| 9 | D | D |
| 11 | C | C |
| 12 | C | n.d. |
| 13 | n.d. | n.d. |
| 14 | C | D |
| 15 | C | D |
| 16 | A | B |
| 17 | A | B |
| 18 | A | D |
| 19 | A | B |
| 20 | A | C |
| 21 | B | n.d. |
| 23 | A | C |
| 24 | D | D |
| 25 | D | D |
| 26 | D | D |
| 27 | D | D |
| 28 | C | D |
| 29 | D | D |
| 30 | D | D |
| 31 | D | D |
| 32 | B | B |
| 33 | B | B |
| 34 | B | C |
| 35 | B | D |
| 36 | C | D |
| 37 | C | D |
| 38 | D | D |
| 39 | D | D |
| 42 | B | D |
| 43 | D | D |
| 44 | C | n.d. |
| 45 | n.d. | n.d. |
| 46 | n.d. | n.d. |
| 47 | n.d. | n.d. |
| 48 | n.d. | n.d. |
| 49 | n.d. | n.d. |
| 61 | B | B |
| 62 | B | C |
| 63 | B | C |
| 64 | B | B |
| 65 | B | C |
| 66 | C | C |
| 67 | C | D |
| 68 | D | D |
| 69 | D | D |
| 77 | C | D |
| 78 | C | D |
| 79 | C | D |
| 80 | C | D |
| 81 | C | n.d. |
| 82 | n.d. | n.d. |
| 83 | n.d. | n.d. |
| 84 | n.d. | n.d. |
| 85 | n.d. | n.d. |
| 86 | n.d. | n.d. |
| 87 | n.d. | n.d. |
| 88 | n.d. | n.d. |
| 89 | n.d. | n.d. |
| 101 | B | B |
| 102 | C | C |
| 103 | B | D |
| 104 | B | B |
| 105 | B | B |
| 106 | B | B |
| 107 | B | C |
| 108 | B | D |
| 109 | B | C |
| 110 | C | n.d. |
| 111 | C | D |
| 112 | C | C |
| 113 | C | C |
| 114 | C | n.d. |
| 115 | C | D |
| 116 | C | D |
| 117 | C | D |
| 118 | C | n.d. |
| 119 | D | n.d. |
| 120 | D | n.d. |
| 121 | D | n.d. |
| 122 | D | n.d. |
| 123 | D | n.d. |
| 124 | D | n.d. |
| 125 | D | n.d. |
| 126 | D | n.d. |
| 127 | D | n.d. |
| 128 | D | n.d. |
| 129 | D | n.d. |
| 130 | D | n.d. |
| 131 | D | D |
| 132 | D | D |
| 133 | n.d | n.d. |
| 134 | D | D |
| 135 | C | D |
| 136 | B | C |
| 137 | B | B |
| 138 | C | C |
| 139 | D | D |

Note:
Microbiological activity data are designated within the following ranges
A: ≤1 μg/mL
B: >1 μg/mL to ≤8.0 μg/mL
C: >8.0 μg/mL to ≤32 μg/mL
D: >32 μg/mL

### III. Preparation of Pharmaceutical Dosage Forms

### Example 1: Oral Capsule

The active ingredient is a compound described herein, or a pharmaceutically acceptable salt thereof. A capsule for oral administration is prepared by mixing 1-1000 mg of active ingredient with starch or other suitable powder blend. The mixture is incorporated into an oral dosage unit, such as a hard gelatin capsule, suitable for oral administration.

We claim:

1. A compound, or a pharmaceutically acceptable salt thereof, having the structure of Formula (I): 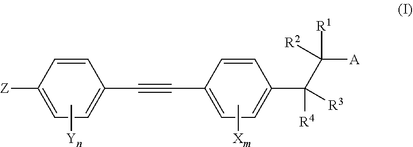
wherein:
A is 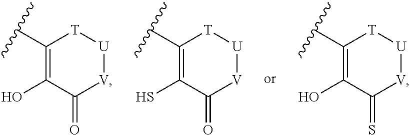
wherein the *-T-U—V— element is selected from the following:
a. *—C(R²¹)═C(R²¹)—N(R²²)—;
b. *—C(R²¹)═C(R²¹)—O—;
c. *—N(R²²)—C(R²¹)═C(R²¹)—;
d. *—N═C(R²¹)—N(R²³)—;
e. *—N═C(R²¹)—O—;
f. *—N(R²²)—N═C(R²¹)—;
g. *—C(R²¹)═N—N(R²²)—;
h. *—C(R²¹)═N—O—; and
i. *—O—C(R²¹)═C(R²¹)—;
each R²¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R²¹ groups together with the carbon atoms to which they are attached join to form a ring;
each R²² is independently H or optionally substituted alkyl; or a R²¹ group and a R²² group on adjacent atoms join to form a ring;
R²³ is optionally substituted alkyl;
n is 0-4;
m is 0-4;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₄-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
provided that when A is 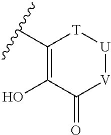
and *-T-U—V is *—O—C(R²¹)═C(R²¹)—, R³ is not H.

2. The compound of claim 1, or a pharmaceutically acceptable salt thereof, wherein the *-T-U—V— element is *—C(R²¹)═C(R²¹)—N(R²²)—.

3. The compound of claim 1, or a pharmaceutically acceptable salt thereof, wherein the *-T-U—V— element is *—C(R²¹)═C(R²¹)—O—.

4. The compound of claim 1, or a pharmaceutically acceptable salt thereof, wherein the *-T-U—V— element is *—N(R²²)—C(R²¹)═C(R²¹)—.

5. The compound of claim 1, or a pharmaceutically acceptable salt thereof, wherein the *-T-U—V— element is *—N═C(R²¹)—N(R²³)—.

6. The compound of claim 1, or a pharmaceutically acceptable salt thereof, wherein the *-T-U—V— element is *—N═C(R²¹)—O—.

7. The compound of claim 1, or a pharmaceutically acceptable salt thereof, wherein the *-T-U—V— element is *—N(R²²)—N═C(R²¹)—.

8. The compound of claim 1, or a pharmaceutically acceptable salt thereof, wherein the *-T-U—V— element is *—C(R²¹)═N—N(R²²)—.

9. The compound of claim 1, or a pharmaceutically acceptable salt thereof, wherein the *-T-U—V— element is *—C(R²¹)═N—O—.

10. The compound of any one of claims 1-9, or a pharmaceutically acceptable salt thereof, wherein A is:

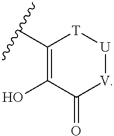

11. The compound of any one of claims 1-9, or a pharmaceutically acceptable salt thereof, wherein A is:

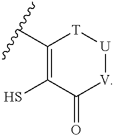

12. The compound of any one of claims 1-9, or a pharmaceutically acceptable salt thereof, wherein A is:

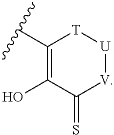

13. A compound, or a pharmaceutically acceptable salt thereof, having the structure of Formula (II): 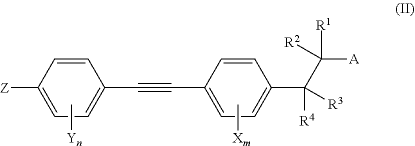
wherein:
A is 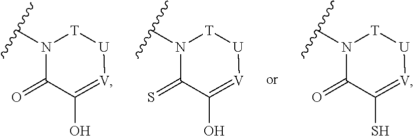
wherein the *-T-U—V— element is selected from the following:
a. *—C(R²¹)═C(R²¹)—C(R²¹)═;
b. *—N═C(R²¹)—C(R²¹)═;
c. *—C(R²¹)═N—C(R²¹)═;
d. *—C(R²¹)═C(R²¹)—N═;
e. *—N═C(R²¹)—N═;
f. *—C(R²¹)═N—N═;
g. *—N═N—C(R²¹)═; and
h. *—N═N—N═;
each R²¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R²¹ groups together with the carbon atoms to which they are attached join to form a ring;
n is 0-4;
m is 0-4;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₄-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

14. The compound of claim 13, or a pharmaceutically acceptable salt thereof, wherein the *-T-U—V— element is *—C(R²¹)═C(R²¹)—C(R²¹)═.

15. The compound of claim 13, or a pharmaceutically acceptable salt thereof, wherein the *-T-U—V— element is *—N═C(R²¹)—C(R²¹)═.

16. The compound of claim 13, or a pharmaceutically acceptable salt thereof, wherein the *-T-U—V— element is *—C(R²¹)═N—C(R²¹)═.

17. The compound of claim 13, or a pharmaceutically acceptable salt thereof, wherein the *-T-U—V— element is *—C(R²¹)═C(R²¹)—N═.

18. The compound of claim 13, or a pharmaceutically acceptable salt thereof, wherein the *-T-U—V— element is *—N═C(R²¹)—N═.

19. The compound of claim 13, or a pharmaceutically acceptable salt thereof, wherein the *-T-U—V— element is *—C(R²¹)═N—N═.

20. The compound of claim 13, or a pharmaceutically acceptable salt thereof, wherein the *-T-U—V— element is *—N═N—C(R²¹)═.

21. The compound of claim 13, or a pharmaceutically acceptable salt thereof, wherein the *-T-U—V— element is *—N═N—N═.

22. The compound of any one of claims 13-21, or a pharmaceutically acceptable salt thereof, wherein A is:

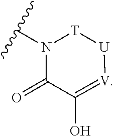

23. The compound of any one of claims 13-21, or a pharmaceutically acceptable salt thereof, wherein A is:

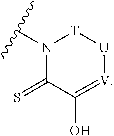

24. The compound of any one of claims 13-21, or a pharmaceutically acceptable salt thereof, wherein A is:

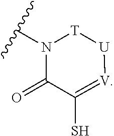

25. A compound, or a pharmaceutically acceptable salt thereof, having the structure of Formula (III): 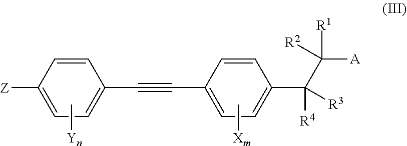
wherein:
A is 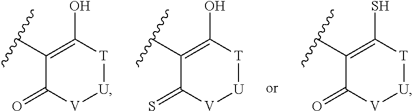
wherein the *-T-U—V— element is selected from the following:
a. *—C(R²¹)═C(R²¹)—N(R²²)—;
b. *—C(R²¹)═C(R²¹)—O—;
c. *—N(R²²)—C(R²¹)═C(R²¹)—;
heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

26. The compound of claim 25, or a pharmaceutically acceptable salt thereof, wherein the *-T-U—V— element is *—C(R²¹)═C(R²¹)—N(R²²)—.

27. The compound of claim 25, or a pharmaceutically acceptable salt thereof, wherein the *-T-U—V— element is *—C(R²¹)═C(R²¹)—O—.

28. The compound of claim 25, or a pharmaceutically acceptable salt thereof, wherein the *-T-U—V— element is *—N(R²²)—C(R²¹)═C(R²¹)—.

29. The compound of claim 25, or a pharmaceutically acceptable salt thereof, wherein the *-T-U—V— element is *—N═C(R²¹)—N(R²²)—.

30. The compound of claim 25, or a pharmaceutically acceptable salt thereof, wherein the *-T-U—V— element is *—N═C(R²¹)—O—.

31. The compound of claim 25, or a pharmaceutically acceptable salt thereof, wherein the *-T-U—V— element is *—N(R²²)—N═C(R²¹)—.

32. The compound of claim 25, or a pharmaceutically acceptable salt thereof, wherein the *-T-U—V— element is *—C(R²¹)═N—N(R²²)—.

33. The compound of claim 25, or a pharmaceutically acceptable salt thereof, wherein the *-T-U—V— element is *—C(R²¹)═N—O—.

34. The compound of any one of claims 25-33, or a pharmaceutically acceptable salt thereof, wherein A is: 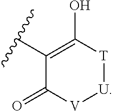
d. *—N═C(R²¹)—N(R²²)—;
e. *—N═C(R²¹)—O—;
f. *—N(R²²)—N═C(R²¹)—;
g. *—C(R²¹)═N—N(R²²)—; and
h. *—C(R²¹)═N—O—;
each R²¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R²¹ groups together with the carbon atoms to which they are attached join to form a ring;
each R²² is independently H or optionally substituted alkyl; or a R²¹ group and a R²² group on adjacent atoms join to form a ring;
n is 0-4;
m is 0-4;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₄-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted

35. The compound of any one of claims 25-33, or a pharmaceutically acceptable salt thereof, wherein A is:

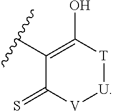

36. The compound of any one of claims 25-33, or a pharmaceutically acceptable salt thereof, wherein A is:

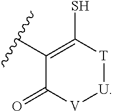

37. The compound of any one of claims 1-36, or a pharmaceutically acceptable salt thereof, wherein each R²¹ is H.

38. The compound of any one of claims 1-36, or a pharmaceutically acceptable salt thereof, wherein each R²¹ is independently H or optionally substituted alkyl.

39. The compound of any one of claims 1-36, or a pharmaceutically acceptable salt thereof, wherein each R²¹ is independently H or optionally substituted alkenyl.

40. The compound of any one of claims 1-36, or a pharmaceutically acceptable salt thereof, wherein each R²¹ is independently H or optionally substituted carbocyclyl.

41. The compound of any one of claims 1-36, or a pharmaceutically acceptable salt thereof, wherein each R²¹ is independently H or optionally substituted carbocyclylalkyl.

42. The compound of any one of claims 1-36, or a pharmaceutically acceptable salt thereof, wherein each R²¹ is independently H or optionally substituted aryl.

43. The compound of any one of claims 1-36, or a pharmaceutically acceptable salt thereof, wherein each R²¹ is independently H or optionally substituted aralkyl.

44. The compound of any one of claims 1-36, or a pharmaceutically acceptable salt thereof, wherein each R²¹ is independently H or optionally substituted heterocyclyl.

45. The compound of any one of claims 1-36, or a pharmaceutically acceptable salt thereof, wherein each R²¹ is independently H or optionally substituted heterocyclylalkyl.

46. The compound of any one of claims 1-36, or a pharmaceutically acceptable salt thereof, wherein each R²¹ is independently H or optionally substituted heteroaryl.

47. The compound of any one of claims 1-36, or a pharmaceutically acceptable salt thereof, wherein each R²¹ is independently H or optionally substituted heteroarylalkyl.

48. The compound of any one of claims 1-36, or a pharmaceutically acceptable salt thereof, wherein two R²¹ groups together with the carbon atoms to which they are attached join to form a ring.

49. The compound of any one of claims 1-48, or a pharmaceutically acceptable salt thereof, wherein R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂.

50. The compound of any one of claims 1-48, or a pharmaceutically acceptable salt thereof, wherein R¹ is unsubstituted alkyl, —COR¹¹, —CON(R¹¹)₂, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, or optionally substituted (C₁-C₄ alkylene)N(R¹²)—CON(R¹¹)₂.

51. The compound of any one of claims 1-48, or a pharmaceutically acceptable salt thereof, wherein R¹ is —COR¹¹, —CON(R¹¹)₂, optionally substituted (C₁-C₄ alkylene)-N(R¹¹)₂, or optionally substituted (C₁-C₄ alkylene)N(R¹²)—COR¹¹.

52. The compound of any one of claims 1-48, or a pharmaceutically acceptable salt thereof, wherein R¹ is —COR¹¹.

53. The compound of any one of claims 1-48, or a pharmaceutically acceptable salt thereof, wherein R¹ is —CON(R¹¹)₂.

54. The compound of any one of claims 1-48, or a pharmaceutically acceptable salt thereof, wherein R¹ is optionally substituted (C₁-C₄ alkylene)-N(R¹¹)₂.

55. The compound of any one of claims 1-48, or a pharmaceutically acceptable salt thereof, wherein R¹ is optionally substituted (C₁-C₄ alkylene)N(R¹²)—COR¹¹.

56. The compound of any one of claims 1-55, or a pharmaceutically acceptable salt thereof, wherein optionally substituted (C₁-C₄ alkylene) is —CH₂—.

57. The compound of any one of claims 1-55, or a pharmaceutically acceptable salt thereof, wherein each R¹¹ is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl.

58. The compound of any one of claims 1-55, or a pharmaceutically acceptable salt thereof, wherein R¹¹ is H.

59. The compound of any one of claims 1-55, or a pharmaceutically acceptable salt thereof, wherein R¹¹ is optionally substituted alkyl.

60. The compound of any one of claims 1-55, or a pharmaceutically acceptable salt thereof, wherein R¹¹ is hydroxy substituted alkyl.

61. The compound of any one of claims 1-55, or a pharmaceutically acceptable salt thereof, wherein R¹¹ is fluoro substituted alkyl.

62. The compound of any one of claims 1-55, or a pharmaceutically acceptable salt thereof, wherein R¹¹ is 2-fluoroethyl.

63. The compound of any one of claims 1-55, or a pharmaceutically acceptable salt thereof, wherein R¹¹ is fluoromethyl.

64. The compound of any one of claims 1-55, or a pharmaceutically acceptable salt thereof, wherein R¹¹ is difluoro substituted alkyl.

65. The compound of any one of claims 1-55, or a pharmaceutically acceptable salt thereof, wherein R¹¹ is 2,2-difluoroethyl.

66. The compound of any one of claims 1-55, or a pharmaceutically acceptable salt thereof, wherein R¹¹ is difluoromethyl.

67. The compound of any one of claims 1-55, or a pharmaceutically acceptable salt thereof, wherein R¹¹ is trifluoro substituted alkyl.

68. The compound of any one of claims 1-55, or a pharmaceutically acceptable salt thereof, wherein R¹¹ is 2,2,2-trifluoroethyl.

69. The compound of any one of claims 1-55, or a pharmaceutically acceptable salt thereof, wherein R¹¹ is trifluoromethyl.

70. The compound of any one of claims 1-32, or a pharmaceutically acceptable salt thereof, wherein R¹¹ is amino substituted alkyl.

71. The compound of any one of claims 1-55, or a pharmaceutically acceptable salt thereof, wherein R¹¹ is cyano substituted alkyl.

72. The compound of any one of claims 1-55, or a pharmaceutically acceptable salt thereof, wherein R¹¹ is optionally substituted alkenyl.

73. The compound of any one of claims 1-55, or a pharmaceutically acceptable salt thereof, wherein R¹¹ is optionally substituted carbocyclyl.

74. The compound of any one of claims 1-55, or a pharmaceutically acceptable salt thereof, wherein R¹¹ is optionally substituted carbocyclylalkyl.

75. The compound of any one of claims 1-55, or a pharmaceutically acceptable salt thereof, wherein R¹¹ is optionally substituted heterocyclyl.

76. The compound of any one of claims 1-55, or a pharmaceutically acceptable salt thereof, wherein R¹¹ is optionally substituted heterocyclylalkyl.

77. The compound of any one of claims 1-55, or a pharmaceutically acceptable salt thereof, wherein R¹¹ is optionally substituted heteroaryl.

78. The compound of any one of claims 1-55, or a pharmaceutically acceptable salt thereof, wherein R¹¹ is optionally substituted heteroarylalkyl.

79. The compound of any one of claims 1-55, or a pharmaceutically acceptable salt thereof, wherein two R¹¹ groups together with the nitrogen to which they are attached join to form an optionally substituted N-heterocyclyl.

80. The compound of any one of claims 1-55, or a pharmaceutically acceptable salt thereof, wherein R¹¹ is vinyl, propan-2-yl, methyl, ethyl, cyclopropyl, cyclopentyl, azentidin-1-yl or allyl.

81. The compound of any one of claims 1-80, or a pharmaceutically acceptable salt thereof, wherein R² is H.

82. The compound of any one of claims 1-81, or a pharmaceutically acceptable salt thereof, wherein R³ is H.

83. The compound of any one of claims 1-82, or a pharmaceutically acceptable salt thereof, wherein R⁴ is H.

84. The compound of any one of claims 1-83, or a pharmaceutically acceptable salt thereof, wherein n is 0 or 1.

85. The compound of any one of claims 1-83, or a pharmaceutically acceptable salt thereof, wherein n is 1 or 2.

86. The compound of any one of claims 1-85, or a pharmaceutically acceptable salt thereof, wherein m is 0 or 1.

87. The compound of any one of claims 1-85, or a pharmaceutically acceptable salt thereof, wherein m is 1 or 2.

88. The compound of any one of claims 1-87, or a pharmaceutically acceptable salt thereof, wherein X is halogen.

89. The compound of any one of claims 1-88, or a pharmaceutically acceptable salt thereof, wherein Y is halogen.

90. The compound of any one of claims 1-89, or a pharmaceutically acceptable salt thereof, wherein Z is -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂.

91. The compound of any one of claims 1-90, or a pharmaceutically acceptable salt thereof, wherein Z is -L-G.

92. The compound of any one of claims 1-89, or a pharmaceutically acceptable salt thereof, wherein Z is morpholinomethyl, (8-oxa-3-azabicyclo[3.2.1]octan-3-yl) methyl, (2-oxa-5-azabicyclo[2.2.1]heptan-5-yl) methyl, (1,1-dioxidothiomorpholino) methyl, (oxetan-3-ylamino) methyl, ((methoxycyclobutyl)amino)methyl, (methylpiperazin-1-yl)methyl, (cyanopyrrolidin-1-yl) methyl, or (methoxypyrrolidin-1-yl) methyl.

93. The compound of any one of claims 1-91, or a pharmaceutically acceptable salt thereof, wherein L is a bond or optionally substituted C₁-C₄ alkylene.

94. The compound of any one of claims 1-91, or a pharmaceutically acceptable salt thereof, wherein L is a bond.

95. The compound of any one of claims 1-91, or a pharmaceutically acceptable salt thereof, wherein L is an optionally substituted C₁-C₄ alkylene.

96. The compound of any one of claims 1-91, or a pharmaceutically acceptable salt thereof, wherein L is optionally substituted C₁.

97. The compound of any one of claims 1-91, or a pharmaceutically acceptable salt thereof, wherein L is —CH₂—.

98. The compound of any one of claims 1-91, or a pharmaceutically acceptable salt thereof, wherein G is optionally substituted alkyl.

99. The compound of any one of claims 1-91, or a pharmaceutically acceptable salt thereof, wherein G is optionally substituted heterocyclyl.

100. The compound of any one of claims 1-91, or a pharmaceutically acceptable salt thereof, wherein G is optionally substituted morpholinyl, piperidinyl, piperazinyl, pyrolidinyl, imidazolyl, imidazolidinyl.

101. The compound of any one of claims 1-91, or a pharmaceutically acceptable salt thereof, wherein G is optionally substituted morpholinyl.

102. The compound of any one of claims 1-91, or a pharmaceutically acceptable salt thereof, wherein G is unsubstituted morpholinyl

103. The compound of any one of claims 1-91, or a pharmaceutically acceptable salt thereof, wherein G is optionally substituted heteroaryl.

104. The compound of any one of claims 1-91 or a pharmaceutically acceptable salt thereof, wherein G is —N(R¹³)₂.

105. The compound of any one of claims 1-91, or a pharmaceutically acceptable salt thereof, wherein G is —OR¹³.

106. The compound of any one of claims 1-91, or a pharmaceutically acceptable salt thereof, wherein G is —CN.

107. The compound of any one of claims 1-106, or a pharmaceutically acceptable salt thereof, wherein each R¹³ is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl.

108. The compound of any one of claims 1-106, or a pharmaceutically acceptable salt thereof, wherein R¹³ is H.

109. The compound of any one of claims 1-106, or a pharmaceutically acceptable salt thereof, wherein R¹³ is optionally substituted alkyl.

110. The compound of any one of claims 1-106, or a pharmaceutically acceptable salt thereof, wherein R¹³ is optionally substituted carbocyclyl.

111. The compound of any one of claims 1-106, or a pharmaceutically acceptable salt thereof, wherein R¹³ is optionally substituted carbocyclylalkyl.

112. The compound of any one of claims 1-106, or a pharmaceutically acceptable salt thereof, wherein R¹³ is optionally substituted heterocyclyl.

113. The compound of any one of claims 1-106, or a pharmaceutically acceptable salt thereof, wherein R¹³ is optionally substituted heterocyclylalkyl.

114. The compound of any one of claims 1-106, or a pharmaceutically acceptable salt thereof, wherein R¹³ is optionally substituted heteroaryl.

115. The compound of any one of claims 1-106, or a pharmaceutically acceptable salt thereof, wherein R¹³ is optionally substituted heteroarylalkyl.

116. The compound of any one of claims 1-106, or a pharmaceutically acceptable salt thereof, wherein two R¹³ groups together with the nitrogen to which they are attached join to form an optionally substituted N-heterocyclyl.

117. A compound, or a pharmaceutically acceptable salt thereof, having the structure of Formula (IV): 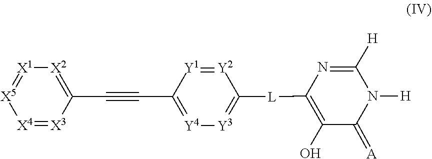
wherein:
A is O or S;
L is *—CR¹R²—CR³R⁴—; *—N(R⁵)—CR³R⁴—; *—CR¹R²—N(R⁵)—; *—O—CR³R⁴—; *—CR⁴R²—O—; *—S—CR³R⁴—; *—CR¹R²—S—; *—N(R⁵)—C(O)—; *—C(O)—N(R⁵)—; *—N(R⁵)—S(O)₂—; *—S(O)₂—N(R⁵)—, or 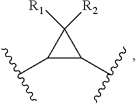
wherein the * denotes a bond to the hydroxypyrimidin-4(3H)-one ring or hydroxypyrimidin-4(3H)-thione ring;
R¹ or R³ are each independently H, optionally alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₀-C₄ alkylene)N(R¹²)—SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
each R⁵ is independently H or optionally substituted alkyl;
each X¹, X², X³, or X⁴ is N, N⁺—O⁻, or C—R⁶;
X⁵ is N, N⁺—O⁻, or C—Z;
each R⁶ is independently H, halogen, —OH, or optionally substituted C₁-C₃ alkyl;
each Y¹, Y², Y³, or Y⁴ is N, N⁺—O⁻, or C—R⁷;
R⁷ is halogen, —OH, or optionally substituted C₁-C₃ alkyl;
Z is H, -L1-G, optionally substituted alkyl, optionally substituted carbocyclyl, optionally substituted aryl, optionally substituted heteroaryl, optionally substituted heterocyclyl;
L1 is a bond, optionally substituted C₁-C₄ alkylene, —C(O)—, or —SO₂—;
G is optionally substituted heterocyclyl, optionally substituted heteroaryl, —N(R¹³)₂, —OR¹³, —CN, —OCON(R¹³)₂, or —N(R¹⁴)CON(R¹³)₂;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
with the provision if L is *—CR¹R²—CR³R⁴—, then at least one of X¹, X², X³, X⁴, X⁵, Y¹, Y², Y³, or Y⁴ is not C.

118. A compound, or a pharmaceutically acceptable salt thereof, having the structure of Formula (V): 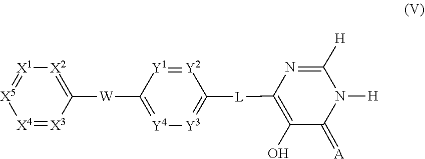
wherein:
A is O or S;
L is *—CR¹R²—CR³R⁴—; *—N(R⁵)—CR³R⁴—; *—CR¹R²—N(R⁵)—; *—O—CR³R⁴—; *—CR⁴R²—O—; *—S—CR³R⁴—; *—CR¹R²—S—; *—N(R⁵)—C(O)—; *—C(O)—N(R⁵)—; *—N(R⁵)—S(O)₂—; *—S(O)₂—N(R⁵)—, or 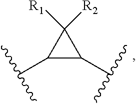
wherein the * denotes a bond to the hydroxypyrimidin-4(3H)-one ring or hydroxypyrimidin-4(3H)-thione ring;
R¹ or R³ are each independently H, optionally alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₀-C₄ alkylene)N(R¹²)—SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
each R⁵ is independently H or optionally substituted alkyl;
each X¹, X², X³, or X⁴ is N, N⁺—O⁻, or C—R⁶;
X⁵ is N, N⁺—O⁻, or C—R⁷;
each R⁶ is independently H, halogen, —OH, or optionally substituted C₁-C₃ alkyl;
each Y¹, Y², Y³, or Y⁴ is N, N⁺—O⁻, or C—R⁷;
R⁷ is halogen, —OH, or optionally substituted C₁-C₃ alkyl;
Z is H, -L1-G, optionally substituted alkyl, optionally substituted carbocyclyl, optionally substituted aryl, optionally substituted heteroaryl, optionally substituted heterocyclyl;
L1 is a bond, optionally substituted C₁-C₄ alkylene, —C(O)—, or —SO₂—;
G is optionally substituted heterocyclyl, optionally substituted heteroaryl, —N(R¹³)₂, —OR¹³, —CN, —OCON(R¹³)₂, or —N(R¹⁴)CON(R¹³)₂;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
W is a bond, —S—, —SO—, —SO₂—, —O—, —CR¹R³—, —C≡C—C≡C—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the X groups.

119. The compound of claim 117 or 118, or a pharmaceutically acceptable salt thereof, wherein A is O.

120. The compound of claim 117 or 118, or a pharmaceutically acceptable salt thereof, wherein L is *—CR¹R²—CR³R⁴—.

121. The compound of claim 117 or 118, or a pharmaceutically acceptable salt thereof, wherein L is *—N(R⁵)—CR³R⁴—.

122. The compound of claim 117 or 118, or a pharmaceutically acceptable salt thereof, wherein L is *—CR¹R²—N(R⁵)—.

123. The compound of claim 117 or 118, or a pharmaceutically acceptable salt thereof, wherein L is *—CR¹R²—O—.

124. The compound of claim 117 or 118, or a pharmaceutically acceptable salt thereof, wherein L is *—CR¹R²—S—.

125. The compound of claim 117 or 118, or a pharmaceutically acceptable salt thereof, wherein L is *—N(R⁵)—C(O)—.

126. The compound of claim 117 or 118, or a pharmaceutically acceptable salt thereof, wherein L is *—C(O)—N(R⁵)—.

127. The compound of claim 117 or 118, or a pharmaceutically acceptable salt thereof, wherein L is *—N(R⁵)—S(O)₂—.

128. The compound of claim 117 or 118, or a pharmaceutically acceptable salt thereof, wherein R¹ or R³ is —COR¹¹.

129. The compound of claim 117 or 118, or a pharmaceutically acceptable salt thereof, wherein R¹ or R³ is —CON(R¹¹)₂.

130. The compound of any one of claims 117-129, or a pharmaceutically acceptable salt thereof, wherein R¹ or R³ is optionally substituted (C₁-C₄ alkylene)-N(R¹¹)₂.

131. The compound of any one of claims 117-129, or a pharmaceutically acceptable salt thereof, wherein R¹ or R³ is optionally substituted (C₁-C₄ alkylene)N(R¹²)—COR¹¹.

132. The compound of any one of claims 117-129, or a pharmaceutically acceptable salt thereof, wherein R¹ or R³ is optionally substituted (C₁-C₄ alkylene)N(R¹²)—CO₂R¹¹.

133. The compound of any one of claims 117-132, or a pharmaceutically acceptable salt thereof, wherein R² is H.

134. The compound of any one of claims 117-133, or a pharmaceutically acceptable salt thereof, wherein R⁴ is H.

135. The compound of any one of claims 117-134, or a pharmaceutically acceptable salt thereof, wherein R⁵ is H.

136. The compound of any one of claims 117-135, or a pharmaceutically acceptable salt thereof, wherein X¹, X² and X³ are CR⁶ and X⁴ is N.

137. The compound of any one of claims 117-135, or a pharmaceutically acceptable salt thereof, wherein X¹, X² and X⁴ are CR⁶ and X³ is N.

138. The compound of any one of claims 117-135, or a pharmaceutically acceptable salt thereof, wherein X¹, X³ and X⁴ are CR⁶ and X² is N.

139. The compound of any one of claims 117-135, or a pharmaceutically acceptable salt thereof, wherein X², X³ and X⁴ are CR⁶ and X¹ is N.

140. The compound of any one of claims 117-135, or a pharmaceutically acceptable salt thereof, wherein X¹ and X² is CR⁶, X³ and X⁴ is N.

141. The compound of any one of claims 117-135, or a pharmaceutically acceptable salt thereof, wherein X¹ and X³ is CR⁶, X² and X⁴ is N.

142. The compound of any one of claims 117-135, or a pharmaceutically acceptable salt thereof, wherein X¹ and X⁴ is CR⁶, X³ and X² is N.

143. The compound of any one of claims 117-135, or a pharmaceutically acceptable salt thereof, wherein X² and X³ is CR⁶, X¹ and X⁴ is N.

144. The compound of any one of claims 117-135, or a pharmaceutically acceptable salt thereof, wherein X² and X⁴ is CR⁶, X¹ and X³ is N.

145. The compound of any one of claims 117-135, or a pharmaceutically acceptable salt thereof, wherein X³ and X⁴ is CR⁶, X¹ and X² is N.

146. The compound of any one of claims 117-135, or a pharmaceutically acceptable salt thereof, wherein X⁵ is C—Z.

147. The compound of any one of claims 117-135, or a pharmaceutically acceptable salt thereof, wherein X⁵ is N.

148. The compound of any one of claims 117-147, or a pharmaceutically acceptable salt thereof, wherein R⁶ is halogen.

149. The compound of any one of claims 117-147, or a pharmaceutically acceptable salt thereof, wherein R⁶ is H.

150. The compound of any one of claims 117-147, or a pharmaceutically acceptable salt thereof, wherein R⁶ is —OH.

151. The compound of any one of claims 117-150, or a pharmaceutically acceptable salt thereof, wherein Y¹, Y² and Y³ are CR⁷ and Y⁴ is N.

152. The compound of any one of claims 117-150, or a pharmaceutically acceptable salt thereof, wherein Y¹, Y² and Y⁴ are CR⁷ and Y³ is N.

153. The compound of any one of claims 117-150 or a pharmaceutically acceptable salt thereof, wherein Y¹, Y³ and Y⁴ are CR⁷ and Y² is N.

154. The compound of any one of claims 117-150, or a pharmaceutically acceptable salt thereof, wherein Y², Y³ and Y⁴ are CR⁷ and Y¹ is N.

155. The compound of any one of claims 117-150, or a pharmaceutically acceptable salt thereof, wherein Y¹ and Y² is CR⁷, Y³ and Y⁴ is N.

156. The compound of any one of claims 117-150, or a pharmaceutically acceptable salt thereof, wherein Y¹ and Y³ is CR⁷, Y² and Y⁴ is N.

157. The compound of any one of claims 117-150, or a pharmaceutically acceptable salt thereof, wherein Y¹ and Y⁴ is CR⁷, Y³ and Y² is N.

158. The compound of any one of claims 117-150, or a pharmaceutically acceptable salt thereof, wherein Y² and Y³ is CR⁷, Y¹ and Y⁴ is N.

159. The compound of any one of claims 117-150, or a pharmaceutically acceptable salt thereof, wherein Y² and Y⁴ is CR⁷, Y¹ and Y³ is N.

160. The compound of any one of claims 117-150, or a pharmaceutically acceptable salt thereof, wherein Y³ and Y⁴ is CR⁷, Y¹ and Y² is N.

161. The compound of any one of claims 117-150, or a pharmaceutically acceptable salt thereof, wherein R⁷ is halogen.

162. The compound of any one of claims 117-150, or a pharmaceutically acceptable salt thereof, wherein R⁷ is H.

163. The compound of any one of claims 117-150, or a pharmaceutically acceptable salt thereof, wherein R⁷ is —OH.

164. The compound of any one of claims 117-163, or a pharmaceutically acceptable salt thereof, wherein Z is H.

165. The compound of any one of claims 117-163, or a pharmaceutically acceptable salt thereof, wherein Z is haloalkyl.

166. The compound of any one of claims 117-163, or a pharmaceutically acceptable salt thereof, wherein Z is hydroxyalkyl.

167. The compound of any one of claims 117-163, or a pharmaceutically acceptable salt thereof, wherein Z is -L1-G.

168. The compound of any one of claims 117-163, or a pharmaceutically acceptable salt thereof, wherein L1 is a bond.

169. The compound of any one of claims 117-163, or a pharmaceutically acceptable salt thereof, wherein L1 is a C₁-C₄ alkylene.

170. The compound of any one of claims 117-163, or a pharmaceutically acceptable salt thereof, wherein L1 is C₁ alkylene.

171. The compound of any one of claims 117-163, or a pharmaceutically acceptable salt thereof, wherein L1 is —CH₂—.

172. The compound of any one of claims 117-163, or a pharmaceutically acceptable salt thereof, wherein G is —N(R¹³)₂—.

173. The compound of any one of claims 117-163, or a pharmaceutically acceptable salt thereof, wherein R¹³ is H.

174. The compound of any one of claims 117-163, or a pharmaceutically acceptable salt thereof, wherein R¹³ is optionally substituted alkyl.

175. The compound of any one of claims 117-163, or a pharmaceutically acceptable salt thereof, wherein R¹³ is hydroxy substituted alkyl.

176. The compound of any one of claims 117-163, or a pharmaceutically acceptable salt thereof, wherein R¹³ is optionally substituted heterocycle.

177. The compound of any one of claims 117-163, or a pharmaceutically acceptable salt thereof, wherein R¹³ is alkoxy substituted heterocycle.

178. The compound of any one of claims 117-163, or a pharmaceutically acceptable salt thereof, wherein R¹³ is optionally substituted cycloalkyl.

179. The compound of any one of claims 117-178, or a pharmaceutically acceptable salt thereof, wherein W is a bond.

180. The compound of any one of claims 117-178, or a pharmaceutically acceptable salt thereof, wherein W is —C≡C—C≡C—.

181. The compound of any one of claims 117-178, or a pharmaceutically acceptable salt thereof, wherein W is *—CR¹═CR³—.

182. The compound of any one of claims 117-178, or a pharmaceutically acceptable salt thereof, wherein W is *—CR¹R²—CR³R⁴—.

183. The compound of any one of claims 117-178, or a pharmaceutically acceptable salt thereof, wherein W is —S—.

184. The compound of any one of claims 117-178, or a pharmaceutically acceptable salt thereof, wherein W is —O—.

185. The compound of any one of claims 117-178, or a pharmaceutically acceptable salt thereof, wherein W is —CR¹R³—.

186. A compound, or a pharmaceutically acceptable salt thereof, having the structure of Formula (VI): 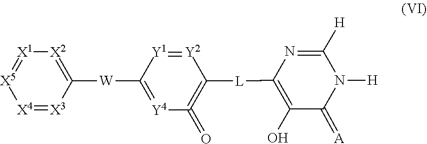
wherein:
A is O or S;
L is *—CR¹R²—CR³R⁴—; *—N(R⁵)—CR³R⁴—; *—O—CR³R⁴—; *—S—CR³R⁴—; *—N(R⁵)—C(O)—; *—C(O)—N(R⁵)—; *—N(R⁵)—S(O)₂—; *—S(O)₂—N(R⁵)—, or 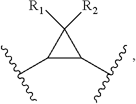
wherein the * denotes a bond to the hydroxypyrimidin-4(3H)-one ring or hydroxypyrimidin-4(3H)-thione ring;
R¹ or R³ are each independently H, optionally alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₀-C₄ alkylene)N(R¹²)—SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
each R⁵ is independently H or optionally substituted alkyl;
each X¹, X², X³, or X⁴ is N, N⁺—O⁻, or C—R⁶;
X⁵ is N, N⁺—O⁻, or C—Z;
each R⁶ is independently H, halogen, —OH, or optionally substituted C₁-C₃ alkyl;
each Y¹, Y², Y³, or Y⁴ is N or C—R⁷;
R⁷ is halogen, —OH, or optionally substituted C₁-C₃ alkyl;
Z is H, -L1-G, optionally substituted alkyl, optionally substituted carbocyclyl, optionally substituted aryl, optionally substituted heteroaryl, optionally substituted heterocyclyl;
L1 is a bond, optionally substituted C₁-C₄ alkylene, —C(O)—, or —SO₂—;
G is optionally substituted heterocyclyl, optionally substituted heteroaryl, —N(R¹³)₂, —OR¹³, —CN, —OCON(R¹³)₂, or —N(R¹⁴)CON(R¹³)₂;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl;
W is a bond, —S—, —SO—, —SO₂—, —O—, —CR⁴R³—, —C≡C—C≡C—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR⁴R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the X groups.

187. The compound of claim 186, or a pharmaceutically acceptable salt thereof, wherein A is O.

188. The compound of claim 186 or 187, or a pharmaceutically acceptable salt thereof, wherein L is *—CR¹R²—CR³R⁴—.

189. The compound of claim 186 or 187, or a pharmaceutically acceptable salt thereof, wherein L is *—N(R⁵)—CR³R⁴—.

190. The compound of claim 186 or 187, or a pharmaceutically acceptable salt thereof, wherein L is *—CR¹R²—N(R⁵)—.

191. The compound of claim 186 or 187, or a pharmaceutically acceptable salt thereof, wherein L is *—CR¹R²—O—.

192. The compound of claim 186 or 187, or a pharmaceutically acceptable salt thereof, wherein L is *—CR¹R²—S—.

193. The compound of claim 186 or 187, or a pharmaceutically acceptable salt thereof, wherein L is *—N(R⁵)—C(O)—.

194. The compound of claim 186 or 187, or a pharmaceutically acceptable salt thereof, wherein L is *—C(O)—N(R⁵)—.

195. The compound of claim 186 or 187, or a pharmaceutically acceptable salt thereof, wherein L is *—N(R⁵)—S(O)₂—.

196. The compound of any one of claims 186-195, or a pharmaceutically acceptable salt thereof, wherein R¹ or R³ is —COR¹¹.

197. The compound of any one of claims 186-195, or a pharmaceutically acceptable salt thereof, wherein R¹ or R³ is —CON(R¹¹)₂.

198. The compound of any one of claims 186-195, or a pharmaceutically acceptable salt thereof, wherein R¹ or R³ is optionally substituted (C₁-C₄ alkylene)-N(R¹¹)₂.

199. The compound of any one of claims 186-195, or a pharmaceutically acceptable salt thereof, wherein R¹ or R³ is optionally substituted (C₁-C₄ alkylene)N(R¹²)—COR¹¹.

200. The compound of any one of claims 186-195, or a pharmaceutically acceptable salt thereof, wherein R¹ or R³ is optionally substituted (C₁-C₄ alkylene)N(R¹²)—CO₂R¹¹.

201. The compound of any one of claims 186-200, or a pharmaceutically acceptable salt thereof, wherein R² is H.

202. The compound of any one of claims 186-201, or a pharmaceutically acceptable salt thereof, wherein R⁴ is H.

203. The compound of any one of claims 186-202, or a pharmaceutically acceptable salt thereof, wherein R⁵ is H.

204. The compound of any one of claims 186-203, or a pharmaceutically acceptable salt thereof, wherein X¹, X² and X³ are CR⁶ and X⁴ is N.

205. The compound of any one of claims 186-203, or a pharmaceutically acceptable salt thereof, wherein X¹, X² and X⁴ are CR⁶ and X³ is N.

206. The compound of any one of claims 186-203, or a pharmaceutically acceptable salt thereof, wherein X¹, X³ and X⁴ are CR⁶ and X² is N.

207. The compound of any one of claims 186-203, or a pharmaceutically acceptable salt thereof, wherein X², X³ and X⁴ are CR⁶ and X¹ is N.

208. The compound of any one of claims 186-203, or a pharmaceutically acceptable salt thereof, wherein X¹ and X² is CR⁶, X³ and X⁴ is N.

209. The compound of any one of claims 186-203, or a pharmaceutically acceptable salt thereof, wherein X¹ and X³ is CR⁶, X² and X⁴ is N.

210. The compound of any one of claims 186-203, or a pharmaceutically acceptable salt thereof, wherein X¹ and X⁴ is CR⁶, X³ and X² is N.

211. The compound of any one of claims 186-203, or a pharmaceutically acceptable salt thereof, wherein X² and X³ is CR⁶, X¹ and X⁴ is N.

212. The compound of any one of claims 186-203, or a pharmaceutically acceptable salt thereof, wherein X² and X⁴ is CR⁶, X¹ and X³ is N.

213. The compound of any one of claims 186-203, or a pharmaceutically acceptable salt thereof, wherein X³ and X⁴ is CR⁶, X¹ and X² is N.

214. The compound of any one of claims 186-203, or a pharmaceutically acceptable salt thereof, wherein X⁵ is C—Z.

215. The compound of any one of claims 186-203, or a pharmaceutically acceptable salt thereof, wherein X⁵ is N.

216. The compound of any one of claims 186-203, or a pharmaceutically acceptable salt thereof, wherein R⁶ is halogen.

217. The compound of any one of claims 186-203, or a pharmaceutically acceptable salt thereof, wherein R⁶ is H.

218. The compound of any one of claims 186-203, or a pharmaceutically acceptable salt thereof, wherein R⁶ is —OH.

219. The compound of any one of claims 186-218, or a pharmaceutically acceptable salt thereof, wherein Y¹ and Y² is CR⁷, Y⁴ is N.

220. The compound of any one of claims 186-218, or a pharmaceutically acceptable salt thereof, wherein Y¹ and Y⁴ is CR⁷, Y² is N.

221. The compound of any one of claims 186-218, or a pharmaceutically acceptable salt thereof, wherein Y² and Y⁴ is CR⁷, Y¹ is N.

222. The compound of any one of claims 186-218, or a pharmaceutically acceptable salt thereof, wherein Y¹ is CR⁷, Y² and Y⁴ is N.

223. The compound of any one of claims 186-218, or a pharmaceutically acceptable salt thereof, wherein Y² is CR⁷, Y¹ and Y⁴ is N.

224. The compound of any one of claims 186-218, or a pharmaceutically acceptable salt thereof, wherein Y⁴ is CR⁷, Y¹ and Y² is N.

225. The compound of any one of claims 186-218, or a pharmaceutically acceptable salt thereof, wherein W is a bond.

226. The compound of any one of claims 186-218, or a pharmaceutically acceptable salt thereof, wherein W is —C≡C—C≡C—.

227. The compound of any one of claims 186-218 or a pharmaceutically acceptable salt thereof, wherein W is *—CR¹═CR³—.

228. The compound of any one of claims 186-218, or a pharmaceutically acceptable salt thereof, wherein W is *—CR¹R²—CR³R⁴—.

229. The compound of any one of claims 186-218 or a pharmaceutically acceptable salt thereof, wherein W is —S—.

230. The compound of any one of claims 186-218, or a pharmaceutically acceptable salt thereof, wherein W is —O—.

231. The compound of any one of claims 186-218, or a pharmaceutically acceptable salt thereof, wherein W is —CR¹R³—.

232. The compound of any one of claims 186-231, or a pharmaceutically acceptable salt thereof, wherein R⁷ is halogen.

233. The compound of any one of claims 186-231, or a pharmaceutically acceptable salt thereof, wherein R⁷ is H.

234. The compound of any one of claims 186-231, or a pharmaceutically acceptable salt thereof, wherein R⁷ is —OH.

235. The compound of any one of claims 186-234, or a pharmaceutically acceptable salt thereof, wherein Z is H.

236. The compound of any one of claims 186-234, or a pharmaceutically acceptable salt thereof, wherein Z is haloalkyl.

237. The compound of any one of claims 186-234, or a pharmaceutically acceptable salt thereof, wherein Z is hydroxyalkyl.

238. The compound of any one of claims 186-234, or a pharmaceutically acceptable salt thereof, wherein Z is -L1-G.

239. The compound of any one of claims 186-234, or a pharmaceutically acceptable salt thereof, wherein L1 is a bond.

240. The compound of any one of claims 186-234, or a pharmaceutically acceptable salt thereof, wherein L1 is a C₁-C₄ alkylene.

241. The compound of any one of claims 186-234, or a pharmaceutically acceptable salt thereof, wherein L1 is C₁ alkylene.

242. The compound of any one of claims 186-234, or a pharmaceutically acceptable salt thereof, wherein L1 is —CH₂—.

243. The compound of any one of claims 186-234, or a pharmaceutically acceptable salt thereof, wherein G is —N(R¹³)₂—.

244. The compound of any one of claims 186-234 or a pharmaceutically acceptable salt thereof, wherein R¹³ is H.

245. The compound of any one of claims 186-234, or a pharmaceutically acceptable salt thereof, wherein R¹³ is optionally substituted alkyl.

246. The compound of any one of claims 186-234, or a pharmaceutically acceptable salt thereof, wherein R¹³ is hydroxy substituted alkyl.

247. The compound of any one of claims 186-234, or a pharmaceutically acceptable salt thereof, wherein R¹³ is optionally substituted heterocycle.

248. The compound of any one of claims 186-234, or a pharmaceutically acceptable salt thereof, wherein R¹³ is alkoxy substituted heterocycle.

249. The compound of any one of claims 186-234 or a pharmaceutically acceptable salt thereof, wherein R¹³ is optionally substituted cycloalkyl.

250. A compound, or a pharmaceutically acceptable salt thereof, having the structure of Formula (VII): 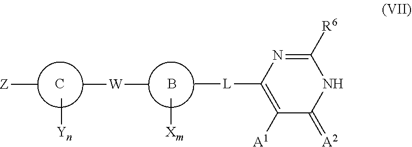
wherein:
n is 0, 1, or 2;
m is 0, 1, or 2;
A¹ is OH or SH;
A² is O or S;
L is a bond, —CR¹R²—, —N(R⁵)—, —O—, —S—, —SO₂—, *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR⁴R²—S—; *—N(R⁵)—C(O)—; *—C(O)—N(R⁵)—; *—N(R⁵)—S(O)₂—; *—S(O)₂—N(R⁵)—, or 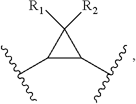
wherein the * denotes a bond to the pyrimidin-4(3H)-one ring or pyrimidin-4(3H)-thione ring;
R¹, R², R³, R⁴, and R⁵ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₀-C₄ alkylene)-CN, optionally substituted (C₀-C₄ alkylene)-OR¹¹, optionally substituted (C₀-C₄ alkylene)-N(R¹¹)₂, optionally substituted (C₀-C₄ alkylene)N(R¹²)—COR¹¹, optionally substituted (C₀-C₄ alkylene)N(R¹²)—CO₂R¹¹, optionally substituted (C₀-C₄ alkylene)N(R¹²)—CON(R¹¹)₂, optionally substituted (C₀-C₄ alkylene)N(R¹²)—SO₂N(R¹¹)₂, optionally substituted (C₀-C₄ alkylene)-O—SO₂N(R¹¹)₂, optionally substituted (C₀-C₄ alkylene)-N(R¹²)—SO₂R¹¹, optionally substituted (C₀-C₄ alkylene)-SO₂R¹¹, or optionally substituted (C₀-C₄ alkylene)N(R¹¹)—PO (optionally substituted C₀-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
R⁶ is H, halogen, optionally substituted alkyl, hydroxyl, alkoxyl, cyano, amino, or nitro; 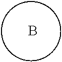
is phenyl, 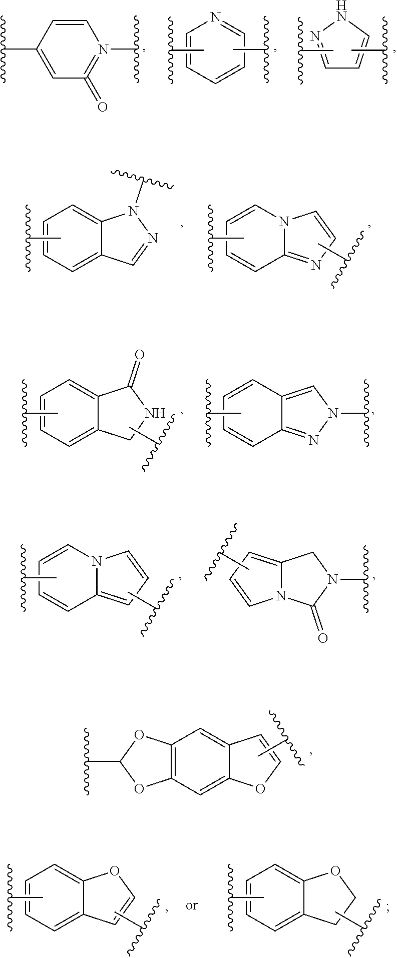
W is a bond, —C≡C—, —C≡C—C≡C—, —S—, —SO—, —SO₂—, —O—, —CR¹R³—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR¹R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the Y groups; 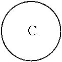
is absent, phenyl, alkyl, cycloalkyl, 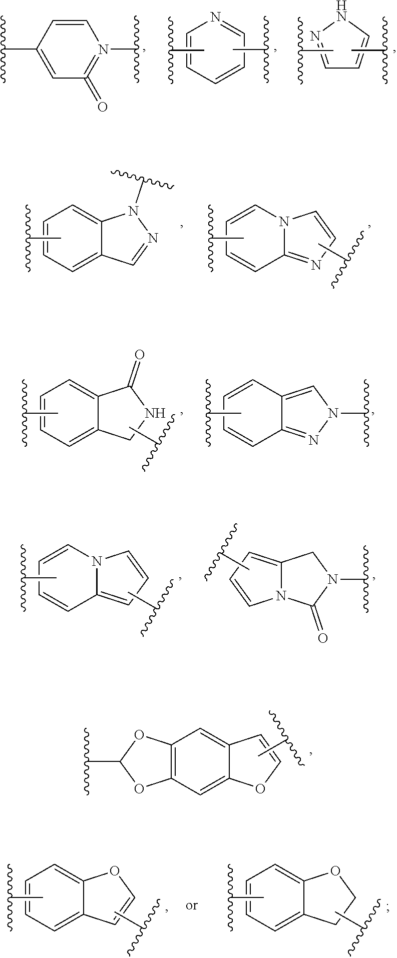
provided that if 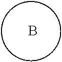
is phenyl or pyridine, then 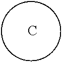
is not phenyl or pyridine; and
if 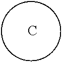
is phenyl or pyridine, then 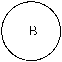
is not phenyl or pyridine;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L2-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L2 is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

251. A compound, or a pharmaceutically acceptable salt thereof, having the structure of Formula (VIIa): 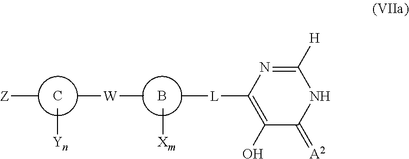
wherein:
n is 0, 1, or 2;
m is 0, 1, or 2;
A² is O or S;
L is —CR¹R²—, —N(R⁵)—, —O—, —S—, —SO₂—, *—CR¹R²—CR³R⁴—; *—N(R⁵)—CR³R⁴—; *—CR¹R²—N(R⁵)—; *—O—CR³R⁴—;
*—CR¹R²—O—; *—S—CR³R⁴—; *—CR¹R²—S—; *—N(R⁵)—C(O)—; *—C(O)—N(R⁵)—; *—N(R⁵)—S(O)₂—; *—S(O)₂—N(R⁵)—: or 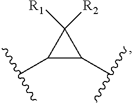
wherein the * denotes a bond to the hydroxypyrimidin-4(3H)-one ring or hydroxypyrimidin-4(3H)-thione ring;
R¹ or R³ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₀-C₄ alkylene)-CN, optionally substituted (C₀-C₄ alkylene)-OR¹¹, optionally substituted (C₀-C₄ alkylene)-N(R¹¹)₂, optionally substituted (C₀-C₄ alkylene)N(R¹²)—COR¹¹, optionally substituted (C₀-C₄ alkylene)N(R¹²)—CO₂R¹¹, optionally substituted (C₀-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₀-C₄ alkylene)N(R¹²)—SO₂N(R¹¹)₂, optionally substituted (C₀-C₄ alkylene)N(R¹²)—SO₂R¹¹, optionally substituted (C₀-C₄ alkylene)-O—SO₂N(R¹¹)₂, or optionally substituted (C₀-C₄ alkylene)N(R¹¹)—PO (optionally substituted C₁-C₄ alkyl)₂;
R², R⁴, and R⁵ are each independently H or optionally substituted C₁-C₃ alkyl; 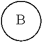
is phenyl, 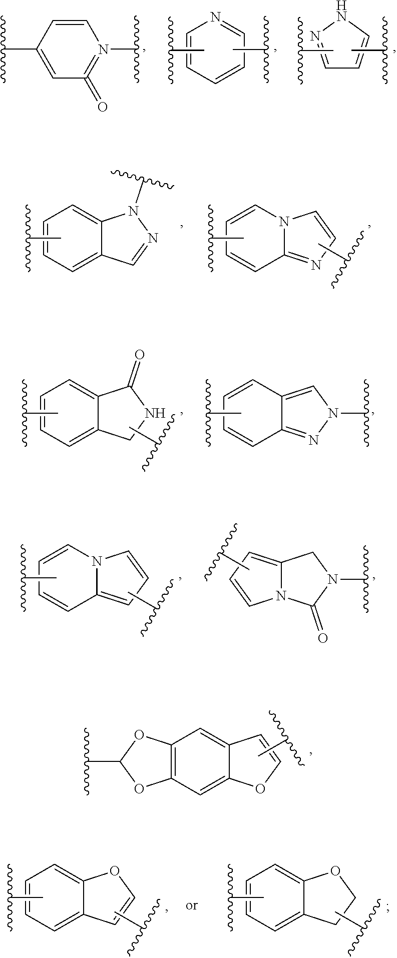
W is a bond, —C≡C—, —C≡C—C≡C—, —S—, —SO—, —SO₂—, —O—, —CR¹R³—, —C(═CR¹H)—, *—CR¹═CR³—; *—CR¹R²—CR³R⁴—; *—N(R⁵)—CR³R⁴—; *—CR¹R²—N(R⁵)—; *—O—CR³R⁴—; *—CR⁴R²—O—; *—S—CR³R⁴—; *—CR⁴R²—S—; *—N(R⁵)—C(O)—; *—C(O)—N(R⁵)—; *—N(R⁵)—S(O)₂—; or *—S(O)₂—N(R⁵)—, wherein the * denotes a bond to the ring having the Y groups; 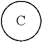
is absent, phenyl, alkyl, cycloalkyl, 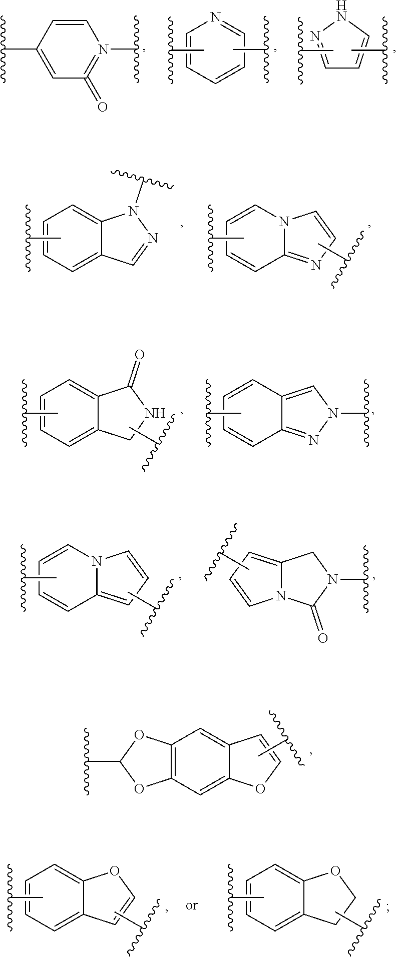
provided that if 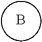
is phenyl or pyridine, then 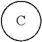
is not phenyl or pyridine; and
if 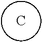
is phenyl or pyridine, then 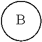
is not phenyl or pyridine;
X is halogen or optionally substituted C₁-C₃ alkyl;
Y is halogen or optionally substituted C₁-C₃ alkyl;
Z is H, optionally substituted alkyl, -L2-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂;
L2 is a bond, optionally substituted C₁-C₄ alkylene, —C(O)—, or —SO₂—;
G is optionally substituted heterocyclyl, optionally substituted heteroaryl, —N(R¹³)₂, —OR¹³, halogen, or —CN;
each R¹¹ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently H or optionally substituted alkyl;
each R¹³ is independently H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently H or optionally substituted alkyl.

252. The compound of either of claims 250 or 251, or a pharmaceutically acceptable salt thereof, wherein R¹ or R³ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₀-C₄ alkylene)-CN, optionally substituted (C₀-C₄ alkylene)-OR¹¹, optionally substituted (C₀-C₄ alkylene)-N(R¹¹)₂, optionally substituted (C₀-C₄ alkylene)N(R¹²)—COR¹¹, optionally substituted (C₀-C₄ alkylene)N(R¹²)—CO₂R¹¹, optionally substituted (C₀-C₄ alkylene)N(R¹²)—CON(R¹¹)₂, optionally substituted (C₀-C₄ alkylene)-SO₂N(R¹¹)₂, optionally substituted (C₀-C₄ alkylene)-SO₂R¹¹, optionally substituted (C₀-C₄ alkylene)N(R¹²)—SO₂N(R¹¹)₂, optionally substituted (C₀-C₄ alkylene)N(R¹²)—SO₂R¹¹, optionally substituted (C₀-C₄ alkylene)-O—SO₂N(R¹¹)₂, or optionally substituted (C₀-C₄ alkylene)N(R¹¹)—PO (optionally substituted C₁-C₄ alkyl)₂.

253. The compound of any one of claims 250-252, or a pharmaceutically acceptable salt thereof, wherein R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)-SO₂N(R¹¹)₂, optionally substituted (C₁-C₄ alkylene)-SO₂R¹¹, or optionally substituted (C₁-C₄ alkylene)N(R¹¹)—PO (optionally substituted C₁-C₄ alkyl)₂.

254. The compound of any one of claims 250-253, or a pharmaceutically acceptable salt thereof, wherein R¹ is optionally substituted heterocyclyl, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)-SO₂N(R¹¹)₂, or optionally substituted (C₁-C₄ alkylene)-SO₂R¹¹.

255. The compound of any one of claims 250-254, or a pharmaceutically acceptable salt thereof, wherein R¹ is —CON(R¹¹)₂, (C₁-C₄ alkylene)-N(R¹¹)₂, or (C₁-C₄ alkylene)N(R¹²)—COR¹¹.

256. The compound of any one of claims 250-255, or a pharmaceutically acceptable salt thereof, wherein R¹ is —CON(R¹¹)₂, (C₁ alkylene)-N(R¹¹)₂, or (C₁ alkylene)N(R¹²)—COR¹¹.

257. The compound of any one of claims 250-256, or a pharmaceutically acceptable salt thereof, wherein R³ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂.

258. The compound of any one of claims 250-257, or a pharmaceutically acceptable salt thereof, wherein R³ and R⁴ are H.

259. The compound of any one of claims 250-258, or a pharmaceutically acceptable salt thereof, wherein R² is H.

260. The compound of any one of claims 250-259, or a pharmaceutically acceptable salt thereof, wherein R¹ and R² are H.

261. The compound of any one of claims 250-260, or a pharmaceutically acceptable salt thereof, wherein n is 0.

262. The compound of any one of claims 250-260, or a pharmaceutically acceptable salt thereof, wherein n is 1 or 2.

263. The compound of any one of claims 250-261, or a pharmaceutically acceptable salt thereof, wherein m is 0.

264. The compound of any one of claims 250-261, or a pharmaceutically acceptable salt thereof, wherein m is 1 or 2.

265. The compound of any one of claims 250-264, or a pharmaceutically acceptable salt thereof, wherein X is halogen.

266. The compound of any one of claims 250-265, or a pharmaceutically acceptable salt thereof, wherein Y is halogen.

267. The compound of any one of claims 250-266, or a pharmaceutically acceptable salt thereof, wherein Z is H, alkyl, -L2-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂.

268. The compound of any one of claims 250-267, or a pharmaceutically acceptable salt thereof, wherein Z is H, alkyl, -L2-G; wherein L2 is a bond, optionally substituted C₁-C₄ alkylene, —C(O)—; and G is optionally substituted heterocyclyl, —N(R¹³)₂, —OR¹³, halogen, or —CN.

269. The compound of any one of claims 250-268, or a pharmaceutically acceptable salt thereof, wherein Z is H, alkyl, or -L2-G, wherein L2 is C₁-C₄ alkylene and G is heterocyclyl.

270. The compound of any one of claims 250-269, or a pharmaceutically acceptable salt thereof, wherein Z is H, alkyl, or -L2-G, wherein L2 is C₁-C₄ alkylene and G is morpholino.

271. The compound of any one of claims 250-270, or a pharmaceutically acceptable salt thereof, wherein each R¹¹ is independently H, haloalkyl, alkyl, alkenyl, carbocyclyl, carbocyclylalkyl; or two R¹¹ groups together with the nitrogen to which they are attached join to form an optionally substituted N-heterocyclyl.

272. The compound of any one of claims 250-270, or a pharmaceutically acceptable salt thereof, wherein each R¹¹ is independently H, haloalkyl, alkyl, or alkenyl.

273. The compound of any one of claims 250-270, or a pharmaceutically acceptable salt thereof, wherein each R¹¹ is independently H, fluoroalkyl, alkyl, or alkenyl.

274. The compound of any one of claims 250-270, or a pharmaceutically acceptable salt thereof, wherein each R¹¹ is independently H, C₁₋₆ fluoroalkyl, C₁₋₆ alkyl, or C₃alkenyl.

275. The compound of any one of claims 250-270, or a pharmaceutically acceptable salt thereof, wherein each R¹¹ is independently H, C₁₋₃ fluoroalkyl, C₁₋₆ methyl, or C₃alkenyl.

276. The compound of any one of claims 250-275, or a pharmaceutically acceptable salt thereof, wherein each R¹³ is independently H, optionally substituted alkyl, optionally substituted carbocyclyl, optionally substituted carbocyclylalkyl, optionally substituted heterocyclyl, optionally substituted heterocyclylalkyl; or two R¹³ groups together with the nitrogen to which they are attached join to form an optionally substituted N-heterocyclyl; and each R¹⁴ is independently H or optionally substituted alkyl.

277. The compound of any one of claims 250-275, or a pharmaceutically acceptable salt thereof, wherein each R¹³ is independently H, alkyl, carbocyclyl, carbocyclylalkyl, heterocyclyl, heterocyclylalkyl; or two R¹³ groups together with the nitrogen to which they are attached join to form an N-heterocyclyl; and each R¹⁴ is H.

278. The compound of any one of claims 250-275, or a pharmaceutically acceptable salt thereof, wherein each R¹³ is independently H, or alkyl; or two R¹³ groups together with the nitrogen to which they are attached join to form an N-heterocyclyl; and each R¹⁴ is H.

279. The compound of any one of claims 250-278, or a pharmaceutically acceptable salt thereof, wherein 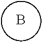
is phenyl,

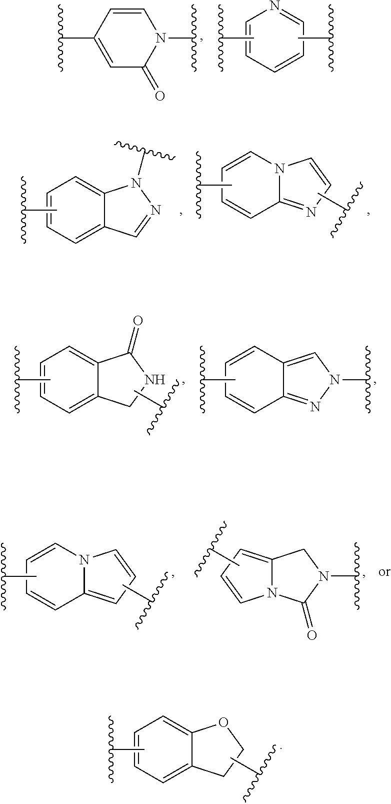

280. The compound of any one of claims 250-279, or a pharmaceutically acceptable salt thereof, wherein 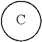
is absent, phenyl, alkyl, cycloalkyl,

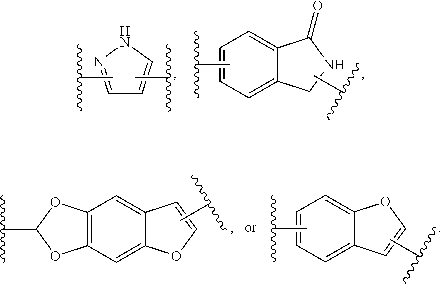

281. The compound of any one of claims 250-279, or a pharmaceutically acceptable salt thereof, wherein 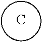
is phenyl, alkyl, cycloalkyl,

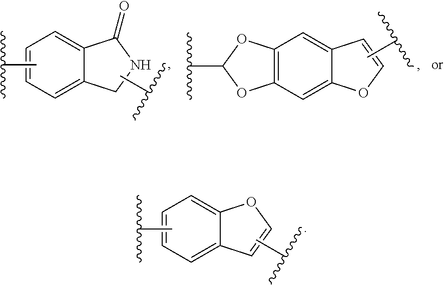

282. The compound of any one of claims 250-279, or a pharmaceutically acceptable salt thereof, wherein 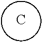
is phenyl or cyclopropyl.

283. The compound of any one of claims 250-282, or a pharmaceutically acceptable salt thereof, wherein W is a bond, —C≡C—, —C≡C—C≡C—, or —CH═CH—.

284. The compound of any one of claims 250-283, or a pharmaceutically acceptable salt thereof, wherein W is a bond, —C≡C—, or —C≡C—C≡C—.

285. A pharmaceutical composition comprising a compound of any one of claims 1-284, or a pharmaceutically acceptable salt thereof, and a pharmaceutically acceptable excipient.

286. A method of inhibiting UDP-{3-O—[(R)-3-hydroxymyristoyl]}-N-acetylglucosamine deacetylase enzyme comprising contacting the enzyme with a compound of any one of claims 1-284.

287. A method for treating bacterial infection in a patient in need thereof comprising administering to the patient a composition of claim 285.
